(19) United States
(12) Patent Application Publication
(10) Pub. No.: US 2024/0109937 A1
(43) Pub. Date: Apr. 4, 2024

(54) BINDING PEPTIDES AND USES THEREOF

(71) Applicant: The Johns Hopkins University, Baltimore, MD (US)

(72) Inventors: Stephen P. Chelko, Baltimore, MD (US); Nazareno Paolocci, Baltimore, MD (US); Daniel P. Judge, Baltimore, MD (US); Gizem Keceli, Baltimore, MD (US); Peter Andersen, Baltimore, MD (US); Nuria Amat, Baltimore, MD (US); Nunzianna Doti, Baltimore, MD (US); Menotti Ruvo, Baltimore, MD (US); Alessandra Monti, Baltimore, MD (US)

(21) Appl. No.: 18/275,586

(22) PCT Filed: Feb. 7, 2022
(86) PCT No.: PCT/US2022/015501

§ 371 Date: Aug. 2, 2023

Related U.S. Application Data
(60) Provisional application No. 63/147,068, filed on Feb. 8, 2021.

(51) Int. Cl. C07K 7/06; A61K 45/06; A61P 9/00; C07K 7/08

(52) U.S. Cl. CPC C07K 7/06; A61K 45/06; A61P 9/00; C07K 7/08; A61K 38/00; C07K 2319/10

(57) ABSTRACT

The invention provides isolated AIF and PPIA binding peptides that disrupt PPIA/AIF complex formation and/or activity and prevent the resulting death of myocardial cells and to pharmaceutical compositions thereof. The invention further provides uses of the isolated peptides in methods of preventing myocardial cell death and/or sudden cardiac death in subject.

### CROSS-REFERENCE TO RELATED APPLICATIONS

This application claims benefit of priority under 35 U.S.C. § 119(e) of U.S. Provisional Application No. 63/147,068, filed Feb. 8, 2021. The disclosure of the prior application is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing text file, name JHU4320_1WO_SL.txt, was created on Feb. 2, 2022 and is 26 kb in size. The file can be accessed using Microsoft Word on a computer that uses Windows OS.

### BACKGROUND OF THE INVENTION

### Field of the Invention

The present invention relates generally to cardiovascular disease and specifically to the use of apoptosis-inducing factor (AIF) and peptidyl-prolyl cis-trans isomerase (PPIA) binding peptides to prevent myocardial cell death and/or sudden cardiac death.

### Background Information

Every year, approximately 400,000-460,000 people die from sudden cardiac death (SCD) in the United States. Arrhythmogenic Cardiomyopathy (ACM) is one of the most arrhythmic forms of heart disease and a leading cause of SCD in young athletes. Clinical features of ACM include ventricular dysfunction and arrhythmias, whereas its most salient pathological traits encompass fibro-fatty replacement of the myocardium and myocyte apoptosis/necrosis. The latter is one of the most cited, yet poorly understood pathological features of ACM, despite its pivotal role in contributing to myocardial fibrosis and subsequent re-entrant ventricular arrhythmia. Furthermore, ACM subjects are particularly at risk of increased disease penetrance and SCD in response to exercise.

Desmoglein-2 (DSG2) is essential to the cardiac desmosome's function and stability, and pathogenic variants in human DSG2 are the second most common cause of ACM. It was previously demonstrated that sedentary homozygous Dsg2 mutant (Dsg2^{mut/mut}) mice recapitulate key ACM phenotypes by early adulthood (16 weeks of age), such as ECG repolarization/depolarization abnormalities, cardiac dysfunction, intercalated disc (ID) remodeling, myocyte injury (inflammation/fibrosis), and calcium (Ca²⁺) mishandling and adiposis. Yet, despite the presence of these functional and pathological cardiac phenotypes (such as extensive biventricular fibrosis), sedentary Dsg2^{mut/mut} mice live well into adulthood and harbor little-to-no apoptotic nuclei at rest. Uniquely, in response to chronic physical effort, Dsg2^{mut/mut} mice experience increased exercised-induced sudden death, while those that survived to exercise endpoint exhibited robust myocardial apoptotic nuclei.

### SUMMARY OF THE INVENTION

The present invention is based on the seminal discovery that AIF and PPIA binding peptides can be used to prevent myocardial cell death and/or sudden cardiac death in patients with arrhythmogenic cardiomyopathy.

In one embodiment, the invention provides an isolated peptide having an amino acid sequence as set forth in Formula I:

Y₁—C—X₁—X₂—X₃—X₄—X₅—X₆—X₇—X₈—X₉—C—Y₂ (Formula I),

wherein: Y₁ is a cell penetrating peptide (CPP), a hydrogen atom or an acetyl group; Y₂ is a CPP or a hydrogen atom; C is a cysteine; X₁ is isoleucine, leucine, valine, terleucine, norleucine, beta-homoleucine, beta-homoisoleucine or 3-amino-5,5-dimethylhexanoic acid in either R or S absolute configuration; X₂ is lysine, histidine, arginine, homoarginine, ornithine, 2,3-diaminopropionic acid, 2,4-diaminobutyric acid or 2-amino-3-guanidinopropionic acid in either R or S absolute configuration; X₃ is leucine, isoleucine, leucine, valine, terleucine, norleucine, beta-homoleucine, beta-homoisoleucine or 3-amino-5,5-dimethylhexanoic acid in either R or S absolute configuration; X₄ is lysine, histidine, arginine, homoarginine, ornithine, 2,3-diaminopropionic acid, 2,4-diaminobutyric acid or 2-amino-3-guanidinopropionic acid in either R or S absolute configuration; X₅ is aspartic acid, isoleucine, leucine, valine, terleucine, norleucine, beta-homoleucine, beta-homoisoleucine, 3-amino-5,5-dimethylhexanoic acid, proline, nipecotic acid, piperidine-2-carboxylic acid, piperidine-4-carboxylic acid, 1,2-dihydro-3(6h)-pyridinone beta-alanine, 2-aminoisobutyric acid, glycine, asparagine, tryptophan or phenylalanine in either R. or S absolute configuration; X₆ is glycine, isoleucine, leucine, valine, terleucine, norleucine, beta-homoleucine, beta-homoisoleucine, 3-amino-5,5-dimethylhexanoic acid, proline, nipecotic acid, piperidine-2-carboxylic acid, piperidine-4-carboxylic acid, 1,2-dihydro-3(6h)-pyridinone beta-alanine, 2 aminoisobutyric acid, glycine, asparagine, tryptophan or phenylalanine in either R or S absolute configuration; X₇ is arginine, histidine, lysine, homoarginine, ornithine, 2,3-diaminopropionic acid, 2,4-diaminobutyric acid or 2-amino-3-guanidinopropionic acid in either R or S absolute configuration; X_{g} is lysine, histidine, arginine, homoarginine, ornithine, 2,3-diaminopropionic acid, 2,4-diaminobutyric acid or 2-amino-3-guanidinopropionic acid in either R or S absolute configuration; and X₉ is valine, isoleucine, leucine, terleucine, norleucine, beta-homoleucine, beta-homoisoleucine or 3-amino-5,5-dimethylhexanoic acid in either R or S absolute configuration; or as set forth in Formula II:

Y₃—X₁₀—X₁₁—X₁₂—X₁₃—X₁₄—X₁₅—X₁₆—X₁₇—X₁₈—X₁₉—X₂—X₂₁—X₂₂—X₂₃—X₂₄—Y₄ (Formula II),

wherein: Y₃ is a CPP, a hydrogen atom or an acetyl group; Y₄ is a CPP, a hydroxyl group or an amino group; X₁₀ is arginine, histidine, lysine, glutamic acid, glutamine, aspartic acid, homoarginine, ornithine, 2,3-diaminopropionic acid, 2,4-diaminobutyric acid or 2-amino-3-guanidinopropionic acid in either R or S absolute configuration; X₁₁ is isoleucine, leucine, valine, terleucine, norleucine, beta-homoleucine, beta-homoisoleucine or 3-amino-5,5-diethylhexanoic acid in either R or S absolute configuration; X₁₂ is isoleucine, leucine, valine, tert-leucine, norleucine, beta-homoleucine, beta-homoisoleucine; or 3-amino-5,5-dimethylhexanoic acid in either R or S absolute configuration; X₁₃ is proline, 1,2,3,4-tetrahydroisoquinoline-3-carboxylic acid or pyroglutamic acid in either R or S absolute configuration; X₁₄ is glycine; X₁₅ is phenylalanine, tyrosine, tryptophan in either R or S absolute configuration; X₁₆ is methionine, cysteine, penicillamine or s-propargyl-cysteine in either R or S absolute configuration; X₁₇ is cysteine, penicillamine, methionine or s-propargyl-cysteine in either R or S absolute configuration; X₁₈ is glutamine, asparagine, glutamic acid or aspartic acid in either R or S absolute configuration; X₁₉ is glycine; X₂₀ is glycine; X₂₁ is aspartic acid, glutamic acid arginine, histidine or lysine in either R or S absolute configuration; X₂₂ is phenylalanine, tryptophan or tyrosine in either R or S absolute configuration; X₂₃ is threonine or serine in either R or S absolute configuration; and X₂₄ is arginine, histidine, lysine, glutamic acid, aspartic acid, glutamine, homoarginine, ornithine, 2,3-diaminopropionic acid, 2,4-diaminobutyric acid or 2-amino-3-guanidinopropionic acid in either R or S absolute configuration

In one aspect, the peptide has an amino acid sequence as set forth in any of SEQ ID NOs:1-60. In another aspect, the isolated peptide includes an N-terminal modification, a C-terminal modification, a detectable label, a cell-penetrating peptide (CPP), a non-natural amino acid, a cyclic peptide, or a combination thereof. In some aspects, the CPP improves cellular uptake, cell penetration and/or transport of the peptide. In various aspects, the CPP is selected from the group consisting of transactivator of transcription (TAT) peptide and TAT peptide variants. In one aspect, the TAT peptide is human immunodeficiency virus TAT. In some aspects, the peptide binds to apoptosis-inducing factor (AIF) and/or to peptidyl-prolyl cis-trans isomerase (PPIA). In other aspects, the peptide is an AIF mimetic peptide. In one aspect, the peptide inhibits Ca²⁺-Calpain-1 (CAPN1)-induced cell death in myocytes. In one aspect, the CAPN1-induced cell death is apoptosis, necrosis, necroptosis, or a combination thereof. In some aspects, the peptide disrupts PPIA/AIF complex formation and/or activity. In various aspects, disrupting PPIA/AIF complex formation and/or activity includes inhibiting AIF binding to PPIA; inhibiting AIF cleavage, oxidation and translocation to the nucleus of myocytes; reducing HMGB1 nuclear export; reducing DNA fragmentation; reducing cell death by necrosis, necroptosis and/or apoptosis, or a combination thereof. In many aspects, the peptide inhibits myocardial cell death.

In another embodiment, the invention provides an isolated nucleic acid sequence encoding a peptide described herein.

In an additional embodiment, the invention provides a pharmaceutical composition including an isolated peptide described herein and a pharmaceutically acceptable carrier.

In one aspect, the pharmaceutically acceptable carrier is selected from the group consisting of phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; sucrose; mannitol; trehalose; sorbitol; sodium; saline; metal surfactants; non-ionic surfactants; polyethylene glycol (PEG); magnesium stearate; water; alcohol; saline solution; glycol; mineral oil and dimethyl sulfoxide (DMSO).

In one embodiment, the invention provides a method of preventing myocardial cell death and/or sudden cardiac death in a subject, including administering to a subject in need thereof a therapeutically effective amount of the pharmaceutical composition described herein, thereby preventing myocardial cell death and/or sudden cardiac death.

In one aspect, the isolated peptide has an amino acid sequence as set forth in any of SEQ ID NOs:1-66. In some aspects, the subject has an inherited or acquired cardiomyopathy. In many aspects, the inherited or acquired cardiomyopathy is arrhythmogenic cardiomyopathy (ACM). In one aspect, the ACM is caused by a mutation in a desmosomal gene. In various aspects, the mutation is a pathogenic variant in a PKP2, DSG2, DSC2, DSP or JUP gene. In various aspects, the method further includes administering to the subject an additional therapeutic treatment. In some aspects, the additional therapeutic treatment includes a beta-blocker, an antiarrhythmic agent, an anticoagulant or an implantable cardioverter-defibrillator.

### BRIEF DESCRIPTION OF THE DRAWINGS

FIGS. 1A-1F illustrate right and left ventricular dysfunction and myocardial necrosis in response to endurance exercise in Dsg2^{mut/mut} mice. FIG. 1A illustrates percent (%) survival during swim. FIG. 1B illustrates representative long-axis (top panel) and 2D, B-mode (bottom two panels) echocardiography from exercised WT and Dsg2^{mut/mut} mice. Dashed box enlarged image of 2D, B-mode middle panel. FIG. 1C illustrates percent RV ejection fraction (% RVEF) vs percent left ventricle EF (% LVEF). FIG. 1D illustrates representative HMGB1 immunostained myocardium from exercised WT mice. FIG. 1E illustrates representative HMGB1 immunostained myocardium from exercised Dsg2^{mut/mut} mice. Arrows; cardiomyocytes (CMs) positive (+) for HMGB 1 nuclear localization; light arrows; non-CMs HMGB1+ nuclei; arrowheads, cytoplasmic HMGB1 localization; dotted-line white boxes highlight enlarged areas (i, ii) on right panels. Light scale bar=100 μm; White scale bars=20 μm. FIG. 1F illustrates myocardium from exercised Dsg2^{mut/mut} mice % HMGB1+ CMs compared to WT mice.

FIGS. 2A-2D illustrate that Dsg2^{mut/mut} mice display ECG abnormalities and myocardial injury in response to endurance exercise. FIG. 2A illustrates representative 90 min signal-averaged ECGs (SAECGs) from exercised mice at swim endpoint. FIG. 2B illustrates exercised Dsg2^{mut/mut} myocardium displayed focal areas of inflammation/inflammatory infiltration as assessed via Hematoxylin & Eosin (H&E). FIG. 2C illustrates Masson's Trichrome stained myocardium from exercised mice. Large scale bar=1 mm; small scale bar=100 μm. Enlarged images are from dotted black boxes. FIG. 2D illustrates Dsg2^{mut/mut} mice % RV and % LV fibrosis compared to WT mice.

FIGS. 3A-3G illustrate that Calpain-1 activation explicates myocyte necrosis. FIG. 3A illustrates representative Calpain-1 (CAPN1) immunoblot from sedentary (rest) and exercised (swim) mice demonstrated presence of total (80 kDa) and active (75 kDa) CAPN1, regardless of genotype, in lysates from exercised mice only. FIG. 3B illustrates exercised Dsg2^{mut/mut} mice total myocardial CAPN1 (80 kDa) expression compared to exercised WT mice. FIG. 3C illustrates active CAPN1 (75 kDa) expression, only observed in myocardial lysates from swimmers, which was potentiated in exercised Dsg2^{mut/mut} mice compared to WT swimmers. FIG. 3D illustrates HL-1 cells incubated in HBS sodium (HBS Na) or HBS potassium/vanadate (HBS KV) media in the absence or presence of 5 mM EGTA; and the Ca²⁺-ionophore, Calcimycin (1 μM, black arrow), to induce calcium (Ca²⁺) overload. FIG. 3E illustrates CAPN1 activity, monitored via proteolytic fluorescent-cleavage of a synthetic peptide (Suc-LLVY-AMC (SEQ ID NO: 81)) in HL-1 cells containing HBS KV media. FIG. 3F illustrates cell death detected via lactose dehydrogenase (LDH) release in the media, in HL-1 cells containing HBS KV. FIG. 3G illustrates representative western blot of Poly [ADP-ribose] polymerase-1 (PARP-1) and the proteolytic fragment of caspase-3 in HL-1 extracts.

FIGS. 4A-4E illustrate Calpain-1 activation and cell death in response to Ca²⁺ overload. FIG. 4A illustrates representative Calpain-2 (CAPN2) immunoblot of myocardial CAPN2 expression in WT and Dsg2^{mut/mut} (Dsg2) mice, in sedentary and exercised cohorts. FIG. 4B illustrates cytosolic Ca²⁺ overload in HL-1 cells loaded with 5 μM Fluo4 FF. FIG. 4C illustrates cell death detected as LDH release. FIG. 4D illustrates CAPN1 activity monitored via proteolytic fluorescent-cleavage of a synthetic CAPN1 peptide (Suc-LLVY-AMC (SEQ ID NO: 81)). FIG. 4E illustrates the relationship between duration of Ca²⁺ overload (x-axis) with CAPN1 activation (%, solid diamonds; left y-axis) and with LDH release (%, solid squares; right y-axis).

FIGS. 5A-5D illustrate reduced calpastatin levels in Dsg2^{mut/mut} mice and rescue of Ca²⁺ overload-induced cell death via CAST-overexpression. FIG. 5A illustrates representative immunoblots probed for changes in calpastatin mitochondrial cytochrome c oxidase subunit IV (COX IV) and calpastatin (CAST) from sedentary (top panel) and exercised (lower panel) mice. Black arrowheads, CAST isoforms; grey arrowheads, CAST proteolytic fragments. FIG. 5B illustrates levels of endogenous CAST isoforms in myocardial lysates from sedentary and exercised Dsg2^{mut/mut} mice compared to WT counterparts. FIG. 5C illustrates expression levels of CAST fragments (90, 65, and 60 kDa) from sedentary and exercised cohorts. FIG. 5D illustrates live-cell imaging of HL-1 cells transfected with a CAST-GFP overexpression construct subjected to HBS KV medium in the absence and presence of Calcimycin (1 μM), to induce Ca²⁺ overload.

FIGS. 6A-6H illustrate calpain-1 inhibition markedly reduces mitochondrial dysfunction and cell death following calcium overload. FIG. 6A illustrates mitochondrial membrane potential (MMP) monitored using the fluorescent ratiometric probe JC-1 (1.5 μM). FIG. 6B illustrates intracellular calcium monitored by means of Fluo4 FF fluorescence. FIG. 6C illustrates CAPN1 activity monitored by proteolysis of the synthetic peptide Suc-LLVY-AMC (SEQ ID NO: 81) (25 μM). FIG. 6D illustrates cell death detected as LDH release. FIG. 6E illustrates representative western blots of CAPN1 or CAPN2 in purified mitochondria from HL-1 cells incubated in HBS medium with sodium (HBS Na), HBS medium with potassium and vanadate (HBS KV), and HBS KV subjected to calcium overload (HBS KVC). FIG. 6F illustrates representative western blots of CAPN1 in purified mitochondria from HL-1 cells subjected to calcium overload (HBS KVC) in the absence or presence of EGTA (5 mM), calpeptin (50 μM), or PD150606 (10 μM). FIG. 6G illustrates representative immunoblots targeting CAPN1 (Domain III) in cytosolic (Cyto.) and mitochondrial bound (Mito. Bound) lysates from exercised (swim) WT and Dsg2^{mut/mut} mice. FIG. 6H illustrates myocardium CAPN1 (Domain III) levels in mitochondrial lysates from exercised Dsg2^{mut/mut} mice.

FIGS. 7A-7D illustrate Calpain-1 inhibition in HL-1 cells by PD150606. FIG. 7A illustrates CAPN1 activity monitored by proteolysis of the synthetic peptide Suc-LLVY-AMC (SEQ ID NO: 81) (25 μM). FIG. 7B illustrates cell death detected as LDH release. FIG. 7C illustrates representative western blots crude and purified fractions of mitochondria were obtained from HL-1 of OPA1, calnexin, Na+/K+-ATPase, LAMP2, β-actin, and MAO-A in protein extracts from total cell homogenate (lane 1), crude mitochondria (lane 2), and purified mitochondria (lane 3). Mito., mitochondria; IMM, inner mitochondrial membrane; Endo. Retic., endoplasmic reticulum; Plasma Memb., plasma membrane; Cytoskel., cytoskeleton; and OMM, outer mitochondrial membrane. FIG. 7D illustrates representative western blots of Calpain-1 (CAPN1, left panel) or Calpain-2 (CAPN2, right panel) in protein extracts from total cell homogenate (lane 1), crude mitochondria (lane 2), and purified mitochondria (lane 3).

FIGS. 8A-8H illustrate exercise increases truncation and nuclear localization of myocardial apoptosis-inducing factor in Dsg2^{mut/mut} mice. FIG. 8A illustrates representative immunoblots of two distinct bands for apoptosis-inducing factor (AIF), a mature (mAIF, 62 kDa), and truncated form (tAIF, 57 kDa), in exercised mice, regardless of genotype. FIG. 8B illustrates Dsg2^{mut/mut} mice myocardial levels of mAIF in response to endurance exercise. FIG. 8C illustrates increased levels of tAIF in response to endurance exercise. FIG. 8D illustrates representative immunoblots from exercised myocardium following subcellular fractionation. Cyto., cytosolic extracts; Mito. Bound, mitochondrial bound extracts; Nucl., nuclear extracts; Chrom. Bound, chromatin-bound extracts. FIG. 8E illustrates levels of cytosolic mAIF in ventricular lysates. FIG. 8F illustrates levels of mito-bound mAIF in ventricular lysates. FIG. 8G illustrates levels nuclear AIF in ventricular lysates. FIG. 8H illustrates levels of chromatin-bound AIF in ventricular lysates.

FIGS. 9A-9D illustrate cytochrome-C in Dsg2^{mut/mut} mice and post-translational modifications of AIF. FIG. 9A illustrates differences in cytochrome-c (cytC) levels observed between cohorts, at rest and in response to exercise (swim). FIG. 9B illustrates differences in the cytosolic-to-mitochondrial bound cytC levels (left panel) or mitochondrial bound-to-cytosolic cytC levels observed in subcellular fractions from exercised cohorts. FIG. 9C illustrates representative myocardial immunoblots from exercised Dsg2^{mut/mut} mice. FIG. 9D illustrates schematic showing immature AIF (iAIF) protein, post-translational processing by MPP (mitochondrial processing peptidase), which promotes binding of mature AIF (mAIF) to the mitochondria inner membrane space. Calpain-cleavage generates truncated AIF (tAIF). MLS, mitochondrial localization signal; TM, transmembrane sequence that binds to mitochondrial intermembrane space; FAD/NADH domains; NLS, nuclear localization signal; M, A, G, and L are amino acids methionine, alanine, glycine, and leucine, respectively. a.a., amino acids; HSP70, heat shock protein-70; PPIA, cyclophilin-A.

FIGS. 10A-10F illustrate CAPN1-mediated truncation of AIF in ACM cardiomyocytes. FIG. 10A illustrates representative immunoblots from WT and Dsg2^{mut/mut} ES-CMs treated for 1 day with either 50 μM isoproterenol (ISO), 1 μM calcium (Ca²⁺), or both ISO and Ca²⁺. FIG. 10B illustrates representative immunoblots from WT and Dsg2^{mut/mut} ES-CMs treated for 7 days with either 50 μM isoproterenol (ISO), 1 μM calcium (Ca²⁺), or both ISO and Ca²⁺. FIG. 10C illustrates representative immunoblots from WT and Dsg2^{mut/mut} ES-CMs treated for 7 days with 50 μM ISO and 1 μM Ca²⁺ (ISO/Ca²⁺), in the absence or presence of 50 μM calpeptin pretreatment. FIG. 10D illustrates calpeptin pre-treatment effect on total AIF (62 kDa) in Dsg2^{mut/mut} ES-CMs compared to WT-treated ES-CMs. FIG. 10E illustrates calpeptin pre-treatment effect on truncated AIF (57 kDa) in Dsg2^{mut/mut} ES-CMs. FIG. 10F illustrates calpeptin pre-treatment effect on active (75 kDa) CAPN1 levels in Dsg2^{mut/mut} ES-CMs.

FIGS. 11A-11G illustrate the increased AIF-nuclear localization in ACM myocardium. FIG. 11A illustrates representative AIF-immunostained myocardium from exercised WT and Dsg2^{mut/mut} mice. Arrowhead, perinuclear-AIF. Scale bar=20 μm. FIG. 11B illustrates AIF/DAPI fluorophore intensity vs fluorophore distribution (α-ω). FIG. 11C illustrates myocardium percent AIF positive (% AIF+) nuclei compared to all cohorts from exercised Dsg2^{mut/mut} mice. FIG. 11D illustrates age at biopsy, explant, or autopsy collection.

FIG. 11E illustrates representative AIF-immunostained myocardia obtained from individuals with no prior history of heart disease (i.e., controls, n=17) (left panel), and AIF-nuclear localization via fluorophore distribution vs. fluorophore intensity at 3-10 regions of interest (α-ω) (right panel). Scale bars=20 μm. FIG. 11F illustrates representative AIF-immunostained myocardia obtained from ACM patients (n=20) (left panel), and AIF-nuclear localization via fluorophore distribution vs. fluorophore intensity at 3-10 regions of interest (α-ω) (right panel). Scale bars=20 μm. FIG. 11G illustrates AIF pathological scores from ACM patients who met Task Force Criteria (TFC) for ACM (phenotype positive; P+) yet harbored no pathogenic desmosomal variant (genotype negative; G−) and patients who met TFC for ACM (P+) and harbored a pathogenic desmosomal variant (G+), compared against age-matched control myocardium.

FIGS. 12A-12H illustrate AIF localization in ACM patient myocardium. FIG. 12A illustrates AIF localization in ACM patient myocardium having an AIF pathological score of Grade 0. FIG. 12B illustrates AIF localization in ACM patient myocardium having an AIF pathological score of Grade 1. FIG. 12C illustrates AIF localization in ACM patient myocardium having an AIF pathological score of Grade 2. FIG. 12D illustrates AIF localization in ACM patient myocardium having an AIF pathological score of Grade 3 or 4. Scale bars=20 m. FIG. 12E illustrates age at biopsy/explant/autopsy collection. FIG. 12F illustrates AIF pathological scores. FIG. 12G illustrates AIF scores in all cohorts.

FIGS. 13A-13I illustrate that exercise fails to up-regulate the mitochondrial thioredoxin-2 system in Dsg2^{mut/mut} hearts. FIG. 13A illustrates schematic of ROS emission recordings via electron paramagnetic resonance (EPR) spectroscopy. FIG. 13B illustrates representative EPR tracings from sedentary (rest) and exercised (swim) cohorts. FIG. 13C illustrates exercised Dsg2^{mut/mut} mice ROS levels in response to exercise. FIG. 13D illustrates western immunoblots from sedentary and exercised cohorts probed for thioredoxin-2 (TXN2), TXN2-reductase (TXNRD2) and peroxiredoxin-3 (PRXD3), normalized to GAPDH. FIG. 13E illustrates sedentary Dsg2^{mut/mut} mice levels of mitochondrial antioxidant protein, TXNRD2, at rest and in response to swimming. FIG. 13F illustrates exercised TXNRD2 levels normalized to sedentary TXNRD2 levels, within genotype. FIG. 13G illustrates western immunoblots from sedentary and exercised cohorts probed for thioredoxin-1 (TXN1) and TXN1-reductase (TXNRD1), normalized to GAPDH. FIG. 13H illustrates sedentary Dsg2^{mut/mut} mice TXNRD1 levels in response to swimming. FIG. 13I illustrates TXNRD1/2 activity assessed in myocardial homogenates.

FIGS. 14A-14C illustrate that Dsg2^{mut/mut} mice display reduced COXIV levels and aberrant myocardial localization. FIG. 14A illustrates representative myocardial immunoblots assessed for changes in mitochondrial cytochrome-c oxidase (COXIV). FIG. 14B illustrates differences in COXIV levels observed in sedentary mice. FIG. 14C illustrates representative COXIV-immunoperoxidase stained myocardia from sedentary and exercised mice.

FIGS. 15A-15F illustrate that exercise promotes AIF-oxidation and DNA fragmentation. FIG. 15A illustrates representative schematic of methoxy polyethylene glycol maleimide (mPEG) treatment. FIG. 15B illustrates representative immunoblots from untreated (−) and 5 kDa mPEG-treated (+) lysates from sedentary (rest) cohorts. FIG. 15C illustrates percent (%) distribution of non-mPEGged and mPEGged AIF levels from sedentary mice. FIG. 15D illustrates representative immunoblots of non-mPEGged and mPEGged mAIF and truncated AIF (tAIF) levels from exercised (swim) cohorts. FIG. 15E illustrates percent distribution of non-mPEGged and mPEGged mAIF and truncated AIF (tAIF) levels from exercised (swim) cohorts. n=6 WT mice/parameter, n=7 Dsg2^{mut/mut} mice/parameter. FIG. 15F illustrates a representative DNA retardation assay.

FIGS. 16A-16B illustrate labeled cysteines and DNA gel electrophoresis. FIG. 16A illustrates representative immunoblot with or without mPEG-treatment. FIG. 16B illustrates representative gel electrophoresis following polymerase chain reaction (PCR) from age-matched littermate DNA samples.

FIGS. 17A-17G illustrate the targeting the PPIA binding site of AIF prevents nuclear import of tAIF, nuclear loss of HMGB1, and AIF-mediated necroptosis. FIG. 17 illustrates representative myocardial immunoblots from exercised WT and DSG2^{mut/mut} mice probed for heat shock protein-70 (HSP70), cyclophilin-A (PPIA) and AIF, normalized to GAPDH. FIG. 17B illustrates Dsg2^{mut/mut} mice HSP70 levels. FIG. 17C illustrates FACS analyses of apoptosis in ISO/Ca²⁺-treated Dsg2^{mut/mut} ES-CMs. FIG. 17D illustrates representative immunoblots from ISO/Ca²⁺-stimulated WT and Dsg2^{mut/mut} ES-CMs treated with 0 μM, 5 μM, or 25 μM AIF-TAT. FIG. 17E illustrates HMGB1 levels in ISO/Ca²⁺-treated Dsg2^{mut/mut} ES-CMs. FIG. 17F illustrates the effect of AIF-TAT mimetic peptide on tAIF nuclear localization. FIG. 17G illustrates representative ES-CMs immunostained for DAPI/AIF/cTnT with corresponding AIF/DAPI fluorophore intensity vs. fluorophore distribution (α-ω) histograms.

FIGS. 18A-18F illustrate HSP70 and PPIA levels in ACM myocytes. FIG. 18A illustrates western immunoblots from sedentary WT and Dsg2^{mut/mut} mice probed for heat shock protein-70 (HSP70), AIF, and cyclophilin-A (PPIA). FIG. 18B illustrates the difference in PPIA levels observed in WT and ACM mice. FIG. 18C illustrates representative immunoblots from ES-CMs treated for 7 days with 50 μM isoproterenol (ISO) and 1 μM calcium (Ca²⁺) probed for PPIA, HSP70, and AIF. FIG. 18D illustrates truncated-AIF (tAIF) formation after 7 days of ISO/Ca²⁺ in both WT and Dsg2^{mut/mut} ES-CMs. FIG. 18E illustrates apoptosis FACS analyses in ISO/Ca²⁺-treated Dsg2^{mut/mut} ES-CMs, in the presence or absence of AIF-TAT mimetic peptide. FIG. 18F illustrates necrosis FACS analyses in ISO/Ca²⁺-treated Dsg2^{mut/mut} ES-CMs, in the presence or absence of AIF-TAT mimetic peptide.

FIGS. 19A-19B illustrate increased AIF-nuclear localization and nuclear loss of HMGB1 in ACM ES-CMs. FIG. 19A illustrates representative immunostained ES-CMs from WT and Dsg2^{mut/mut} ES-CMs stimulated with ISO/Ca²⁺ for 7 days stained for AIF, cTnT, and DAPI. Cross, AIF+ nuclei. FIG. 19B illustrates representative immunostained ES-CMs from WT and Dsg2^{mut/mut} ES-CMs stimulated with ISO/Ca²⁺ for 7 days stained for HMGB1, cTnT, and DAPI. Arrows, HMGB1+ nuclei. Arrowheads, nuclear loss of HMGB1.

FIG. 20 illustrates a schematic representation of exercise-induced, CAPN1/PPIA-mediated AIF-nuclear import in Arrhythmogenic Cardiomyopathy.

### DETAILED DESCRIPTION OF THE INVENTION

The present invention is based on the seminal discovery that AIF and PPIA binding peptides can be used to prevent myocardial cell death and/or sudden cardiac death in patients with arrhythmogenic cardiomyopathy.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein, which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides an isolated peptide having an amino acid sequence as set forth in Formula I:

Y₁—C—X₁—X₂—X₃—X₄—X₅—X₆—X₇—X₈—X₉—C—Y₂ (Formula I),

wherein: Y₁ is a cell penetrating peptide (CPP), a hydrogen atom or an acetyl group; Y₂ is a CPP or a hydrogen atom; C is a cysteine; X₁ is isoleucine, leucine, valine, terleucine, norleucine, beta-homoleucine, beta-homoisoleucine or 3-amino-5,5-dimethylhexanoic acid in either R or S absolute configuration; X₂ is lysine, histidine, arginine, homoarginine, ornithine, 2,3-diaminopropionic acid, 2,4-diaminobutyric acid or 2-amino-3-guanidinopropionic acid in either R or S absolute configuration; X₃ is leucine, isoleucine, leucine, valine, terleucine, norleucine, beta-homoleucine, beta-homoisoleucine or 3-amino-5,5-dimethylhexanoic acid in either R or S absolute configuration; X₄ is lysine, histidine, arginine, homoarginine, ornithine, 2,3-diaminopropionic acid, 2,4-diaminobutyric acid or 2-amino-3-guanidinopropionic acid in either R or S absolute configuration; X₅ is aspartic acid, isoleucine, leucine, valine, terleucine, norleucine, beta-homoleucine, beta-homoisoleucine, 3-amino-5,5-dimethylhexanoic acid, proline, nipecotic acid, piperidine-2-carboxylic acid, piperidine-4-carboxylic acid, 1,2-dihydro-3(6h)-pyridinone beta-alanine, 2-aminoisobutyric acid, glycine, asparagine, tryptophan or phenylalanine in either R or S absolute configuration; X₆ is glycine, isoleucine, leucine, valine, terleucine, norleucine, beta-homoleucine, beta-homoisoleucine, 3-amino-5,5-dimethylhexanoic acid, proline, nipecotic acid, piperidine-2-carboxylic acid, piperidine-4-carboxylic acid, 1,2-dihydro-3(6h)-pyridinone beta-alanine, 2 aminoisobutyric acid, glycine, asparagine, tryptophan or phenylalanine in either R or S absolute configuration; X₇ is arginine, histidine, lysine, homoarginine, ornithine, 2,3-diaminopropionic acid, 2,4-diaminobutyric acid or 2-amino-3-guanidinopropionic acid in either R or S absolute configuration; X_{g} is lysine, histidine, arginine, homoarginine, ornithine, 2,3-diaminopropionic acid, 2,4-diaminobutyric acid or 2-amino-3-guanidinopropionic acid in either R or S absolute configuration; and X₉ is valine, isoleucine, leucine, terleucine, norleucine, beta-homoleucine, beta-homoisoleucine or 3-amino-5,5-dimethylhexanoic acid in either R or S absolute configuration; or as set forth in Formula II:

Y₃—X₁₀—X₁₁—X₁₂—X₁₃—X₁₄—X₁₅—X₁₆—X₁₇—X₁₈—X₁₉—X₂—X₂₁—X₂₂—X₂₃—X₂₄—Y₄ (Formula II),

wherein: Y₃ is a CPP, a hydrogen atom or an acetyl group; Y₄ is a CPP, a hydroxyl group or an amino group; X₁₀ is arginine, histidine, lysine, glutamic acid, glutamine, aspartic acid, homoarginine, ornithine, 2,3-diaminopropionic acid, 2,4-diaminobutyric acid or 2-amino-3-guanidinopropionic acid in either R or S absolute configuration; X₁₁ is isoleucine, leucine, valine, terleucine, norleucine, beta-homoleucine, beta-homoisoleucine or 3-amino-5,5-dimethylhexanoic acid in either R or S absolute configuration; X₁₂ is isoleucine, leucine, valine, tert-leucine, norleucine, beta-homoleucine, beta-homoisoleucine; or 3-amino-5,5-dimethylhexanoic acid in either r or s absolute configuration; X₁₃ is proline, 1,2,3,4-tetrahydroisoquinoline-3-carboxylic acid or pyroglutamic acid in either R or S absolute configuration; X₁₄ is glycine; X₁₅ is phenylalanine, tyrosine, tryptophan in either R or S absolute configuration; X₁₆ is methionine, cysteine, penicillamine or s-propargyl-cysteine in either R or S absolute configuration; X₁₇ is cysteine, penicillamine, methionine or s-propargyl-cysteine in either R or S absolute configuration; X₁₈ is glutamine, asparagine, glutamic acid or aspartic acid in either R or S absolute configuration; X₁₉ is glycine; X₂₀ is glycine; X₂₁ is aspartic acid, glutamic acid arginine, histidine or lysine in either R or S absolute configuration; X₂₂ is phenylalanine, tryptophan or tyrosine in either R or S absolute configuration; X₂₃ is threonine or serine in either R or S absolute configuration; and X₂₄ is arginine, histidine, lysine, glutamic acid, aspartic acid, glutamine, homoarginine, ornithine, 2,3-diaminopropionic acid, 2,4-diaminobutyric acid or 2-amino-3-guanidinopropionic acid in either R or S absolute configuration.

The terms “peptide”, “polypeptide” and “protein” are used interchangeably herein and refer to any chain of at least two amino acids linked by a covalent chemical bond. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The coding sequence boundaries are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The peptides described herein are binding peptides, that can bind to and potentially block the interaction between apoptosis-inducing factor (AIF) and peptidyl-prolyl cis-transisomerase (PPIA).

Apoptosis-inducing factor (AIF) is a protein that triggers chromatin condensation and DNA fragmentation in a cell in order to induce programmed cell death. AIF is a flavoprotein that also acts as an NADH oxidase, and which is involved in initiating a caspase-independent pathway of apoptosis (positive intrinsic regulator of apoptosis) by causing DNA fragmentation and chromatin condensation. AIF also regulates the permeability of the mitochondrial membrane upon apoptosis. Normally it is found behind the outer membrane of the mitochondrion and is therefore secluded from the nucleus. However, when the mitochondrion is damaged, it moves to the cytosol and to the nucleus. Inactivation of AIF leads to resistance of embryonic stem cells to death following the withdrawal of growth factors indicating that it is involved in apoptosis. The activity of AIF depends on the type of cell, the apoptotic insult, and its DNA-binding ability. AIF also plays a significant role in the mitochondrial respiratory chain and metabolic redox reactions.

Peptidylprolyl isomerase A (PPIA) also known as cyclophilin A (CypA) is an enzyme, and a member of the peptidyl-prolyl cis-trans isomerase (PPIase) family; this protein catalyzes the cis-trans isomerization of proline imidic peptide bonds, which allows it to regulate many biological processes, including intracellular signaling, transcription, inflammation, and apoptosis. Proline has an unusually conformationally restrained peptide bond due to its cyclic structure with its side chain bonded to its secondary amine nitrogen. Most amino acids have a strong, energetic preference for the trans peptide bond conformation due to steric hindrance, but proline's unusual structure stabilizes the cis form so that both isomers are populated under biologically relevant conditions. Proline is unique among the natural amino acids in having a relatively small difference in free energy between the cis configuration of its peptide bond and the more common trans form. The activation energy required to catalyze the isomerization between cis and trans is relatively high: ˜2 kcal/mol (c.f. ˜0 kcal/mol for regular peptide bonds). Unlike regular peptide bonds, the X-prolyl peptide bond will not adopt the intended conformation spontaneously; thus, the process of cis-trans isomerization can be the rate-limiting step in the process of protein folding. Prolyl isomerases such as PPIA, therefore, function as protein folding chaperones. Cis peptide bonds N-terminal to proline residues are often located at the first residue of certain types of tight turns in the protein backbone. Proteins that contain structural cis prolines in the native state include ribonuclease A, ribonuclease Ti, beta lactamase, cyclophilin, and some interleukins. As described herein, PPIA acts as a nuclear chaperone for AIF to ensure its nuclear import from the mitochondria.

ATF and PPIA binding peptides have been initially designed as corresponding to the protein's regions involved in mutual interaction on both the AIF and PPIA side of the complex interface (see Table 1).

TABLE 1
AIF-based peptides known to bind CypA and displace its binding to AIF.

| Compound | Features of the molecule | Description |
|---|---|---|
| AIF | Linear peptide, 25 amino | Design and characterization of AIF (370- |
| (370-394) | acids long which: | 394). Characterization of AIF (370- |
| | 1. disrupts the complex | 394)/CypA interaction in terms of K_{D}. |
| | AIF/Cyclophilin in living | Evaluation of the cellular effects of AIF |
| | cells and in vitro with | (370-394) peptide in HT-22 cells. |
| | an IC50 of 5 μM. | NMR-based molecular model of AIF/CypA |
| | 2. binds CypA in a region | protein. Validation of the model by using the |
| | overlapping with that of | peptide AIF (370-394). Molecular model of |
| | AIF protein, with a K_{D} | AIF (370-394)/CypA complex. |
| | of around 5 μM. | |
| | 3. inhibits the nuclear | |
| | translocation of AIF/CypA | |
| | complex in HT-22 cells. | |
| | 4. does not inhibit the | |
| | cis-trans prolyl isomerase | |
| | activity of CypA, | |
| | like AIF protein. | |
| | Ac-QSVGVSSGKLLIKLK_{D}GRKV | |
| | ETDHI-NH₂ | |
| | (SEQ ID NO: 61) | |
| TAT-AIF | Linear peptide, 38 amino | In vivo effects of AIF-TAT mimetic peptide. |
| (370-394) | acids long. Bears an N- | |
| | terminal TAT fragment | |
| | for cell uptake. Disrupts the | |
| | complex AIF/Cyclophilin | |
| | with a K_{D} of around 5 μM | |
| | and prevents brain injury | |
| | in neonatal mice after | |
| | hypoxia-ischemia. | |
| | NH₂-GRKKRRQRRR(βAla)QSVG | |
| | VSSGKLLIKLKDGRKVETDHI-NH₂ | |
| | (SEQ ID NO: 62) | |
| AIF | Bi-cyclic peptide, 25 | Identification and characterization of a new |
| (370-394)- | amino acids long. Can be N- | stapled AIF (370-394) analogs. |
| stapled | terminally modified with | The peptide is cyclized through a triazole |
| peptides | TAT fragment for cell | bridge connecting K(N3) and hpG, where |
| | uptake. Disrupts the | K(N3) stands for N-s-azidolysine and hpG |
| | complex AIF/Cyclophilin | stands for 1-homopropargylglycine and |
| | with a K_{D} of around 0.8 μM. | through a disulfide bridge between the two |
| | The peptide is slightly | cysteines reported in bold, italics and |
| | soluble. | underlined. |
| | Ac-QSCGVSSG[K(N3)]LLCKL | |
| | K_{D}GRKV[hpG]TDHI-NH₂ | |
| | (SEQ ID NO: 63) | |
| CypA (55- | Linear peptide, 15 amino | Design, synthesis and biochemical |
| 69) | acids long. Can be N- | characterization of the peptide mimicking the |
| | terminally modified with | binding region of AIF on CypA. This peptide |
| | TAT fragment for cell | has not been used in cells, to date. |
| | uptake. Disrupts the | |
| | complex AIF/Cyclophilin | |
| | with a K_{D} of around 6 μM. | |
| | To date the effects of this | |
| | peptide in cells has not | |
| | been evaluated. | |
| | Ac-RIIPGFMCQGGDFTR-NH₂ | |
| | (SEQ ID NO: 64) | |
| AIF (381- | Cyclic peptide, 11 amino | Design, synthesis and characterization of a |
| 389) ox | acids long. Can be N- | shorter cyclic AIF (370-394) analog (AIF |
| | terminally modified with | (381-389) ox). |
| | TAT fragment for cell | |
| | uptake. It disrupts the | |
| | complex AIF/Cyclophilin with | |
| | a K_{D} of around 2 μM and | |
| | provides neuroprotective | |
| | effects in HT-22 cells | |
| | during the oxidative stress. | |
| | Ac-CIKLK_{D}GRKVC-NH₂ | |
| | (SEQ ID NO: 65) | |
| AIF (381- | Cyclic peptide, 11 amino | Design, synthesis and characterization of a |
| 389) LLox | acids long. Can be N- | shorter cyclic AIF (381-389) ox analog (AIF |
| | terminally modified wit | (381-389) LLox), with improved affinity. |
| | TAT fragment for cell | |
| | uptake and containing | |
| | the dual mutation DG->LL. | |
| | It disrupts the complex | |
| | AIF/Cyclophilin with a K_{D} of | |
| | around 1 μM and provides | |
| | neuroprotective effects in | |
| | HT-22 cells during the | |
| | oxidative stress. | |
| | Ac-CIKLKLLRKVC-NH₂ | |
| | (SEQ ID NO: 66) | |

The peptides of the present invention, which can bind AIF and/or PPIA were designed based on a progenitor peptide: CIKLKDGRKVC (SEQ ID NO:67), and can be described by the following general formula:

Y₁—C—X₁—X₂—X₃—X₄—X₅—X₆—X₇—X₈—X₉—C—Y₂ (Formula I),

The single-letter sequence IKLKDGRKV (SEQ ID NO:68) corresponds to region 381-389 of AIF, while “C” stands for cysteine residues which are intended to be connected by a disulfide bridge formed between the thiol's side chains, thus forming a cyclic peptide. The N- and C-termini are described by the same Y₁ and Y₂ of Formula (I).

In Formula (I):

X₁ is isoleucine (I) or leucine (L) or valine (V) or terleucine (Tle) or norleucine (Nle) or beta-homoleucine (hPL) or beta-homoisoleucine (hβI); or 3-amino-5,5-dimethylhexanoic acid in either R or S absolute configuration.

X₂ is lysine (K) or histidine (H) or arginine (R) or homoarginine (hR) or ornithine (Orn) or 2,3-diaminopropionic acid (DAP) or 2,4-diaminobutyric acid (DAB) or 2-amino-3-guanidinopropionic acid in either R or S absolute configuration.

X₃ is leucine or isoleucine or leucine or valine or terleucine or norleucine or beta-homoleucine or beta-homoisoleucine; or 3-amino-5,5-dimethylhexanoic acid in either R or S absolute configuration.

X₄ is lysine or histidine, or arginine or homoarginine or ornithine or 2,3-diaminopropionic acid or 2,4-diaminobutyric acid or 2-amino-3-guanidinopropionic acid in either R or S absolute configuration.

X₅ is aspartic acid (D) or isoleucine or leucine or valine or terleucine or norleucine or beta-homoleucine or beta-homoisoleucine; or 3-amino-5,5-dimethylhexanoic acid or proline or nipecotic acid or piperidine-2-carboxylic acid or piperidine-4-carboxylic acid or 1,2-dihydro-3(6H)-pyridinone beta-alanine or 2-aminoisobutyric acid (Aib) or glycine (G) or asparagine (N) or tryptophan (W) or phenylalanine (F) in either R or S absolute configuration.

X₆ is glycine or isoleucine or leucine or valine or terleucine or norleucine or beta-homoleucine or beta-homoisoleucine; or 3-amino-5,5-dimethylhexanoic acid or proline or nipecotic acid or piperidine-2-carboxylic acid or piperidine-4-carboxylic acid or 1,2-dihydro-3(6H)-pyridinone beta-alanine or 2 aminoisobutyric acid or glycine or asparagine or tryptophan or phenylalanine in either R or S absolute configuration.

X₇ is arginine or histidine, or Lysine or homoarginine or ornithine or 2,3-diaminopropionic acid or 2,4-diaminobutyric acid or 2-amino-3-guanidinopropionic acid in either R or S absolute configuration.

Xₛ is lysine or histidine, or arginine or homoarginine or ornithine or 2,3-diaminopropionic acid or 2,4-diaminobutyric acid or 2-amino-3-guanidinopropionic acid in either R or S absolute configuration.

X₉ is valine or isoleucine or leucine or terleucine or norleucine or beta-homoleucine or beta-homoisoleucine; or 3-amino-5,5-dimethylhexanoic acid in either R or S absolute configuration.

Y₁ represents a cell penetrating peptide (CPP) attached to the peptide N-terminus through its C-terminus forming an amide bond. A spacer group can be conveniently inserted in between the CPP and the peptide molecule. CPP sequences can be chosen among those described for example in the literature (Habault J, Poyet J L. Recent Advances in Cell Penetrating Peptide-Based Anticancer Therapies. Molecules. 2019; 24(5):927); a hydrogen atom and an acetyl group. When Y₁ is a hydrogen group it means that a free amino group is occurring at the peptide N-terminus, as it is well-known to those skilled in the art.

Y₂ can be similarly a CPP attached through its N-terminus to the peptide C-terminus via an amide bond. A spacer group can be conveniently inserted in between the CPP and the peptide molecule. Y₂ can also be a hydroxyl group or an amino group. When Y₂ is a hydroxyl group it means that the peptide has a carboxylic group at its C-terminus. When Y₂ is an amino group it means that the peptide has an amide group at its C-terminus.

Peptides can be derived by those described by Formula (I) changing the method of cyclization. Methods for appropriate peptide cyclization are for example those reported in Joon-Seok Choi and Sang Hoon Joo. Recent Trends in Cyclic Peptides as Therapeutic Agents and Biochemical Tools. Biomol Ther (Seoul). 2020 January; 28(1): 18-24; which can be here applied to obtain molecules having cyclic molecules of similar ring size.

A first exemplary set of peptides is reported in Table 2 together with the values of KDs determined.

TABLE 2
Exemplary set of cyclic peptides derived from Formula (I).
Only the residues changed following the description of Formula
(I) are reported. In all these molecules Y₁ can a hydrogen atom,
accounting for a free N-terminus. Y₂ can an amino group,
accounting for a C-terminal amide.

| ENTRY | KD (μM) |
|---|---|
| X₂: hR | 80.28 |
| X₄: hR | >10³ |
| X₈: hR | >10³ |
| X₂; X₃; X₈: hR | 13.96 |
| X₅-X₆: L-L | 1.20 |
| X₅-X₆: Tle-L | 146.8 |
| X₅-X₆: L- Tle | 151.2 |
| X₅-X₆: Tle-Tle | 13.63 |
| X₅-X₆: L-V | 7.57 |
| X₅-X₆: V-L | 37.61 |
| X₅-X₆: V-V | 268.2 |
| X₅-X₆: Nle -L | 30.67 |
| X₅-X₆: L- Nle | 23.90 |
| X₅-X₆: Nle-Nle | 22.94 |
| X₅-X₆: I-L | 89.88 |
| X₅-X₆: L-I | 233.4 |
| X₅-X₆: I-I | 58.92 |

A further non-exhaustive set of exemplary peptides derived from Formula (I) and designed on the basis of the sequence of AIF (381-389) is provided herein (SEQ ID NOs: 1-30). All peptides are intended as cyclized through a disulfide bridge connecting the N- and C-terminal cysteines' side chains. In all these molecules Y₁ can be a hydrogen atom, accounting for a free N-terminus. Y₂ can be an amino group, accounting for a C-terminal amide.

Additional peptides of the present invention, which can bind AIF were designed based on a progenitor peptide: RIIPGFMCQGGDFTR (SEQ ID NO:69), and can be described by the following general formula:

Y₃—X₁₀—X₁₁—X₁₂—X₁₃—X₁₄—X₁₅—X₁₆—X₁₇—X₁₈—X₁₉—X₂—X₂₁—X₂₂—X₂₃—X₂₄—Y₄ (Formula II),

The single-letter sequence RIIPGFMCQGGDFTR (SEQ ID NO:69) corresponds to region 55-69 of CypA. The N- and C-termini are described by the same Y₃ and Y₄ of the following Formula (II).

In Formula (II):

X₁₀ is arginine (R) or histidine (H) or lysine (K) or glutamic acid (E) or glutamine (Q) or aspartic acid (D) or homoarginine (hR) or ornithine (Orn) or 2,3-diaminopropionic acid or 2,4-diaminobutyric acid or 2-amino-3-guanidinopropionic acid in either R or S absolute configuration.

X₁₁ is isoleucine (I) or leucine (L) or valine (V) or terleucine (Tle) or Norleucine (Nle) or beta-homoleucine (hPL) or beta-homoisoleucine (hβI) or 3-amino-5,5-dimethylhexanoic acid in either R or S absolute configuration.

X₁₂ is isoleucine or leucine or valine or terleucine or norleucine or beta-homoleucine or beta-homoisoleucine; or 3-amino-5,5-dimethylhexanoic acid in either R or S absolute configuration.

X₁₃ is proline (P) or 1,2,3,4-tetrahydroisoquinoline-3-carboxylic acid or pyroglutamic acid (PCA) in either R or S absolute configuration.

X₁₄ is glycine (G).

X₁₅ is phenylalanine (F) or tyrosine (Y) or tryptophan (W) in either R or S absolute configuration.

X₁₆ is methionine (M) or cysteine (C) or penicillamine or s-propargyl-cysteine (SPRC) in either R or S absolute configuration.

X₁₇ is cysteine or penicillamine or methionine or s-propargyl-cysteine in either R or S absolute configuration.

X₁₈ is glutamine or asparagine or glutamic acid or aspartic acid in either R or S absolute configuration.

X₁₉ is glycine.

X₂₀ is glycine.

X₂₁ is aspartic acid (D) or glutamic acid arginine or histidine or Lysine in either R or S absolute configuration.

X₂₂ is phenylalanine or tryptophan or tyrosine in either R or S absolute configuration.

X₂₃ is threonine (T) or serine (S) in either R or S absolute configuration.

X₂₄ is arginine or histidine or lysine or glutamic acid (E) or aspartic acid (D) or glutamine (Q) or homoarginine or ornithine or 2,3-diaminopropionic acid or 2,4-diaminobutyric acid or 2-amino-3-guanidinopropionic acid in either R or S absolute configuration.

Y₃ represents a CPP attached to the peptide N-terminus through its C-terminus forming an amide bond. A spacer group can be conveniently inserted in between the CPP and the peptide molecule. CPP sequences can also be a hydrogen atom, an acetyl group. When Y₃ is a hydrogen group, a free amino group occurs at the peptide N-terminus, as it is well-known to those skilled in the art.

Y₄ can be similarly a CPP; attached through its N-terminus to the peptide C-terminus via an amide bond. A spacer group can be conveniently inserted in between the CPP and the peptide molecule. Y₄ can also be a hydroxyl group or an amino group. When Y₄ is a hydroxyl group it means that the peptide has a carboxylic group at its C-terminus. When Y₄ is an amino group it means that the peptide has an amide group at its C-terminus.

A further non-exhaustive set of exemplary peptides derived from Formula (II) and designed based on the sequence of CypA (55-69) is provided herein (SEQ ID NOs:31-60). In all these molecules, Y₃ can be a hydrogen atom, accounting for a free N-terminus. Y₄ can be an amino group, accounting for a C-terminal amide.

In one aspect, the peptide has an amino acid sequence as set forth in any of SEQ ID NOs:1-60.

TABLE 3
List of AIF and PPIA binding
peptides of the invention

| Sequence | SEQ ID NO |
|---|---|
| CLKLKLLRKVC | SEQ ID NO: 1 |
| CLKIKLLRKVC | SEQ ID NO: 2 |
| CLKIKLLRKVC | SEQ ID NO: 3 |
| CLKIKLLRKIC | SEQ ID NO: 4 |
| CLKIKLLRK(Tle)C | SEQ ID NO: 5 |
| C(Tle)K(Tle)KLLRK(Tle)C | SEQ ID NO: 6 |
| C(Tle)KLKLLRK(Tle)C | SEQ ID NO: 7 |
| C(Tle)KLKLLRKVC | SEQ ID NO: 8 |
| C(Tle)(hR)L(hR)LL(hR)KVC | SEQ ID NO: 9 |
| C(hBL)KLKLLRKVC | SEQ ID NO: 10 |
| C(hßL)K(hBL)KLLRKVC | SEQ ID NO: 11 |
| C(hBL)K(hBL)KLLRK(hBL)C | SEQ ID NO: 12 |
| C(hBL)KLKLLRKVC | SEQ ID NO: 13 |
| C(hBI)K(hBI)KLLRKVC | SEQ ID NO: 14 |
| C(hBI)K(hBI)KLLRK(hBI)C | SEQ ID NO: 15 |
| C(Nle)KLKLLRKVC | SEQ ID NO: 16 |
| C(Nle)K(Nle)KLLRKVC | SEQ ID NO: 17 |
| C(Nle)K(Nle)KLLRK(Nle)C | SEQ ID NO: 18 |
| CI(Orn)LKLLRKVC | SEQ ID NO: 19 |
| CI(Orn)L(Orn)LLRKVC | SEQ ID NO: 20 |
| CI(Orn)L(Or)LL(Orn)KVC | SEQ ID NO: 21 |
| CI(Orn)L(Or)LL(Orn) | SEQ ID NO: 22 |
| (Or)VC | |
| CIHLKLLRKVC | SEQ ID NO: 23 |
| CIHLHLLRKVC | SEQ ID NO: 24 |
| CIHLHLLHKVC | SEQ ID NO: 25 |
| CIHLHLLHHVC | SEQ ID NO: 26 |
| CIKLK(Aib)LRKVC | SEQ ID NO: 27 |
| CIKLK(Aib)(Aib)RKVC | SEQ ID NO: 28 |
| CIKLKL(Aib)RKVC | SEQ ID NO: 29 |
| CIKLKPPRKVC | SEQ ID NO: 30 |
| RLLPGFMCQGGDFTR | SEQ ID NO: 31 |
| RIIPGFMCQGGDFT(hR) | SEQ ID NO: 32 |
| (hR)IIPGFMCQGGDFT(hR) | SEQ ID NO: 33 |
| RIIPGFMCQGGDFTH | SEQ ID NO: 34 |
| RIIPGFMCQGGDFTK | SEQ ID NO: 35 |
| RIIPGFMCEGGDFTK | SEQ ID NO: 36 |
| RIIPGFMCEGGDFTK | SEQ ID NO: 37 |
| RIIPGFM(SPRC)EGGDFTK | SEQ ID NO: 38 |
| RIIPGFMCQGGDWTR | SEQ ID NO: 39 |
| RIIPGWMCQGGDWTR | SEQ ID NO: 40 |
| RIIPGWMCQGGDFTR | SEQ ID NO: 41 |
| RIIPGWMCQGGDWTR | SEQ ID NO: 42 |
| KIIPGWMCQGGDWTE | SEQ ID NO: 43 |
| KIIPGFMCQGGDFTE | SEQ ID NO: 44 |
| (Orn)IIPGFMCQGGDFTD | SEQ ID NO: 45 |
| R(Nle)(Nle)PGFMCQGGDFTR | SEQ ID NO: 46 |
| RI(Nle)PGFMCQGGDFTR | SEQ ID NO: 47 |
| R(Tle)(Tle)PGFMCQGGDFTR | SEQ ID NO: 48 |
| RII(PCA)GFMCQGGDFTR | SEQ ID NO: 49 |
| KII(PCA)GFMCQGGDFTE | SEQ ID NO: 50 |
| KII(PCA)GFMCQGGDFTD | SEQ ID NO: 51 |
| KIIPGWMCQGGDWTD | SEQ ID NO: 52 |
| (Orn)IIPGFMCQGGDFTE | SEQ ID NO: 53 |
| (Orn)IIPGWMCQGGDWTE | SEQ ID NO: 54 |
| (Orn)IIPGWMCQGGDWTD | SEQ ID NO: 55 |
| KIIPGWMCQGGDWTD | SEQ ID NO: 56 |
| HIIPGFMCQGGDFTH | SEQ ID NO: 57 |
| HIIPGWMCQGGDWTH | SEQ ID NO: 58 |
| KIIPGFMCQGGDFTK | SEQ ID NO: 59 |
| KIIPGWMCQGGDWTK | SEQ ID NO: 60 |

In another aspect, the isolated peptide includes an N-terminal modification, a C-terminal modification, a detectable label, a cell-penetrating peptide (CPP), a non-natural amino acid, a cyclic peptide, or a combination thereof.

Virtually any modification of the peptides of the invention can be performed, and is included in the present disclosure, as long as it is not detrimental to the properties of the peptide (the impact of a modification of a peptide on its activity can be routinely assessed, and modeling tools can be used to predict the impact of the modification of the peptides on their activity). Alternatively, small cleavable components can be incorporated if there are any concerns regarding a loss of activity due to the position of a peptide modification (such as a CPP, for example).

Non-limiting examples of N-terminal modification that can be introduced at the N-terminal extremity of the isolated peptide of the invention include: 5-FAM, 5-FAM-Ahx, Abz, acetylation, Acryl, Alloc, Benzoyl, Biotin, Biotin-Ahx, BOC, Br—Ac—, BSA (—NH2 of N terminal), CBZ, Dansyl, Dansyl-Ahx, Decanoic acid, DTPA, Fatty Acid, FITC, FITC-Ahx, Fmoc, Formylation, Hexanoic acid, HYNIC, KLH (—NH2 of N terminal), Lauric acid, Lipoic acid, Maleimide, MCA, Myristoyl, Octanoic acid, OVA (—NH2 ofN terminal), Palmitoyl, PEN, Stearic acid, Succinylation, and TMR.

Non-limiting examples of C-terminal modification that can be introduced at the C-terminal extremity of on the isolated peptide of the invention include: AFC, AMC, Amidation, BSA (—COOH of C terminal), Bzl, Cysteamide, Ester (OEt), Ester (OMe), Ester (OtBu), Ester (OTBzl), KLH (—COOH of C terminal), MAPS Asymmetric 2 branches, MAPS Asymmetric 4 branches, MAPS Asymmetric 8 branches, Me, NHEt, NHisopen, NHMe, OSU, OVA (—COOH of C terminal), p-Nitroanilide, and tBu.

“Cyclic peptides” are polypeptide chains that contain a circular sequence of bonds through a connection between the amino and carboxyl ends of the peptide, a connection between the amino end and a side chain, or two side chains or more complicated arrangements. Even though most cyclic peptides are membrane-impermeable, some cyclic peptides have unique features that allow cell entry by passive diffusion, endocytosis, endosomal escape, or other mechanisms. Therefore, cyclic peptides can also be used as conjugate peptides, to improve cell permeability of a peptide of the invention (also referred to as cargo peptide).

The peptides of the invention can be labeled in various ways to allow their detection and/or distinction from other peptides. The peptides can, for example, be labeled by the incorporation of stable radioisotopes in amino acids. Non-limiting examples of radiolabeled amino acids include: Arg (¹³C₆, ¹⁵N₄), Ile (¹³C₆, ¹⁵N), Leu (¹³C₆, ¹⁵N), Lys (¹³C₆, ¹⁵N₂), and Val(¹³C₅, ¹⁵N). Peptides can also be labeled by the incorporation of non-conventional or non-natural amino acids.

The terms “unnatural amino acids”, “non-natural amino acids” and “non-naturally occurring amino acids” can be used interchangeably and refer to non-proteinogenic amino acids that either occur naturally or are chemically synthesized. They can be used as building blocks, conformational constraints, molecular scaffolds, or pharmacologically active products and represent a nearly infinite array of diverse structural elements for the development of peptidic and non-peptidic compounds. Non-limiting examples of non-naturally occurring amino acids include p-amino acids (β3 and β2), homo-amino acids, proline and pyruvic acid derivatives, 3-substituted alanine derivatives, glycine derivatives, ring-substituted phenylalanine and tyrosine derivatives, linear core amino acids, N-methyl amino acids, alicyclic amino acids, arginine derivatives, aromatic amino acids, asparagine derivatives, aspartic acid derivatives, cysteine derivatives, DAB (2,4-Diaminobutyric Acid), DAP (2,3-Diaminopropionic Acid), glutamic acid derivatives, glutamine derivatives, isoleucine derivatives, leucine derivatives, lysine derivatives, methionine derivatives, norleucine derivatives, norvaline derivatives, ornithine derivatives, penicillamine derivatives, phenylalanine derivatives, phenylglycine derivatives, pyroglutamine derivatives, serine —unnatural amino acids and derivatives, threonine derivatives, tryptophan derivatives, tyrosine derivatives and valine derivatives.

The peptides can also be labeled by the addition of a fluorescent label or tag to the amino acid sequence of the peptide. As used herein, the terms “fluorescent peptide”, “fluorescent tag”, “fluorophore” and the like are interchangeable. Non-limiting examples of fluorescent label or tag include: 1-pyrenemethylamine HCL, 5-FAM (N-Terminal), 5-FAM-Ahx (N-Terminal), Abz/DNP, Abz/Tyr (3-NO2), DABCYL, DABCYL/Glu(EDANS)-NH2, Dansyl (N-Terminal), Dansyl-Ahx (N-Terminal), EDANS/DABCYL, FITC (N-Terminal), FITC-Ahx (N-Terminal), Glu (EDANS)-NH2, MCA (N-Terminal), MCA/DNP, quenched fluorescent peptide, Tyr (3-NO2), TMR, AMC, CF, TAMRA, RhB, MCA, NBD, PBA, BODIPY, fragmented BODIPY.

The peptides can also be labeled by the incorporation of a chemiluminescent label, such as luciferin or luminol; or by the incorporation of a member of a donor/acceptor pair, such as mClover3/mRuby3, EBFP2/mEGFP, ECFP/EYFP, Cerulean/Venus, MiCy/mKO, CyPet/YPet, EGFP/mCherry, Venus/mCherry, Venus/tdTomato, and Venus/mPlum for example.

Additional modifications of the peptide can include peptide cyclization by creating disulfide bridges between cysteine residues on the peptide, phosphorylation, methylation, PEGylation, and multiple antigens peptide (MAP) application, and any additional modification of a peptide known in the art.

In some aspects, the isolated peptide can be modified to have improved overall stability, extended blood stream stability, improved cell permeability, improved cellular activity, or a combination thereof, as compared to an unmodified peptide. Such modification can be the result of the addition of a cell-penetrating peptide (CPP). In some aspects, the CPP improves cellular uptake, cell penetration and/or transport of the peptide.

In some aspects, the CPP improves cellular uptake, cell penetration and/or transport of the peptide.

As used herein “improved” stability, cell permeability or cellular activity is meant to refer to the stability, cell permeability or cellular activity of the peptide that is increased, ameliorated or augmented when the peptide is modified, as compared to the same peptide without such modification. As used herein, the peptide's blood stream stability refers to the amount of time the peptide stays in the blood stream, which can be measured by evaluating the peptide half-life, for example. An “extended” stability of a modified peptide indicates that the peptide can be detected in the blood stream for more extended periods when it is modified compared to when it is not.

The peptide of the invention can, for example, include a short polypeptide sequences, such as a CPP, which efficiently transports biologically active molecule inside living cells, and improves cellular uptake of the peptide of interest. The peptide's cellular uptake can be measured, for example, as the ratio of cytosol versus extracellular concentration of the peptide.

CPP sequences are well known in the art. For example, a CPP sequence can be chosen among those described in the literature (Habault J, Poyet J L. Recent Advances in Cell Penetrating Peptide-Based Anticancer Therapies. Molecules. 2019; 24(5):927); a hydrogen atom and an acetyl group.

In other aspects, the CPP is selected from the group consisting of penetratin, Tat peptide, Tat peptide variants, pVEC, chimeric transportan, MPG peptide, linear and cyclic polyarginines, R₈, R₉, R₆W₃, EB1, VP22, model amphipathic peptide (MAP), Pep-1 and Pep-1 related peptides, fusion sequence-based protein (FBP), transportan analog7 (TP-7), TP-9, TP-10, azurin and azurin derivatives, protamine, protamine-fragment/SV40 peptides, polyethylenimine (PEI), poly-lysine, histidine-lysine peptides, poly-arginine, and gp41 fusion sequence.

CPP and protein transduction domains (PTD) are well known in the art for their ability to efficiently transport biologically active molecules inside living cells. CPP are typically less than 30 residues in length (see Table 4) and often carry a positive charge. The CPP or cyclic peptide and the cargo peptide can be covalently conjugated or physically complexed through non-covalent interaction by bulk-mixing of the CPP and the cargo. Each CPP has physicochemical properties, a preferred mode of administration, a specific barrier, and a preferred target cell, which need to be taken into account when pairing a CPP to a cargo protein. Further modification of the CPP or cyclic peptide, such as Na-methylation can be used to increase the permeabilization of the peptide.

TABLE 4
Non- exhaustive list of sequences of CPPs

| CPP | Sequence | SEQ ID NO: |
|---|---|---|
| Penetratin | RQIKIWFQNRRMKWKK | SEQ ID NO: 70 |
| Tat peptide | GRKKRRQRRRPPQ | SEQ ID NO: 71 |
| pVEC | LLIILRRRIRKQ | SEQ ID NO: 72 |
| | AHAHSK | |
| chimeric | GWTLNSAGYLLGKI | SEQ ID NO: 73 |
| transportan | NLKALAALAKKIL | |
| MPG peptide | GALFLGFLGAAGST | SEQ ID NO: 74 |
| | MGAWSPKKKRKV | |
| Pep-1 | KETWWETWWTEWSQ | SEQ ID NO: 75 |
| | PKKKRKV | |
| synthetic | | SEQ ID NO: 82 |
| polyarginines | | |
| (Rₙ; 6 ≤ n ≤ 12) | | |
| R₈ | RRRRRRRR | SEQ ID NO: 76 |
| R₉ | RRRRRRRRR | SEQ ID NO: 77 |
| MAP | KLALKLALKA | SEQ ID NO: 78 |
| | LKAALKLA | |
| R₆W₃ | RRWWRRWRR | SEQ ID NO: 79 |
| P28 | LSTAADMQGVVTDG | SEQ ID NO: 80 |
| (azurin 50-77) | MASGLDK_{D}YLKPDD | |

In various aspects, the CPP is selected from the group consisting of transactivator of transcription (TAT) peptide and TAT peptide variants. In one aspect, the TAT peptide is human immunodeficiency virus TAT.

In some aspects, a spacer group can be conveniently inserted in between a CPP and the peptide molecule.

As used herein, the term “spacer” or “linker” can be used interchangeably to refer to any bond, small molecule, or other vehicle which allows the CPP and the peptide to be physically linked. A linker can be any chemical moiety that is capable of linking two peptides (such as a CPP and a binding peptide) in a stable, covalent manner.

The peptide described herein can bind to AIF and/or to PPIA. In one aspect, the peptide is an AIF mimetic peptide.

As used herein, the term “mimetic peptide” or “peptidomimetic” refers to a small protein-like chain designed to mimic a peptide. Mimetic peptide typically arises either from modification of an existing peptide or by designing similar systems that mimic peptides. Irrespective of the approach, the altered chemical structure is designed to advantageously adjust the molecular properties such as stability or biological activity, especially for the development of drug-like compounds from existing peptides. These modifications involve changes to the peptide that will not occur naturally (such as altered backbones and the incorporation of non-natural amino acids).

The peptide described herein can be an AIF mimetic peptide; therefore, the peptide can include a similar function as an AIF peptide (e.g., binds to PPIA to induce caspase-independent apoptosis by causing DNA fragmentation and chromatin condensation) but present advantageous properties as compared to an AIF peptide. For example, an AIF mimetic peptide can have increased stability, increased binding specificity and/or sensitivity to PPIA, resistance to degradation, or a combination thereof.

In one aspect, the peptide inhibits Ca²⁺-Calpain-1 (CAPN1)-induced cell death in myocytes.

Calpains, such as Calpain-1 (CAPN1), are calcium-activated neutral proteases, non-lysosomal, intracellular cysteine proteases. Mammalian calpains include ubiquitous, stomach-specific, and muscle-specific proteins. As detailed below in the Example section, the present invention is in part based on the discovery that in myocytes, CAPN1, upon activation by Ca²⁺, such as by a Ca²⁺ overload, can be imported into mitochondria (because of mitochondria membrane depolarization), where it can cleave AIF to generate truncated AIF (tAIF). tAIF can then be subjected to oxidation and translocation into myocytes nucleus (via its nuclear chaperone PPIA), where it can induce a release of HMGB1 from the nucleus to the cytoplasm.

High mobility group box 1 protein (HMGB1), like histones, is among the most important chromatin proteins. In the nucleus, HMGB1 interacts with nucleosomes, transcription factors, and histones to organize the DNA and regulate transcription. After binding, HMGB1 bends DNA, which facilitates the binding of other proteins. HMGB1 supports the transcription of many genes in interactions with many transcription factors. It also interacts with nucleosomes to loosen packed DNA and remodel the chromatin. The presence of HMGB1 in the nucleus depends on posttranslational modifications. When the protein is not acetylated, it stays in the nucleus, but hyperacetylation on lysine residues causes it to translocate into the cytosol.

Therefore, Ca²⁺ overload in myocytes can induce CAPN1 activation, CAPN1 mitochondrial translocation, AIF cleavage, AIF oxidation, tAIF translocation in the nucleus, and HMGB1 nuclear export, which can, in turn, be responsible for the DNA fragmentation, which can trigger cellular apoptosis, necrosis and/or necroptosis, i.e., Ca²⁺-CAPN1-induced cell death.

In one aspect, the CAPN1-induced cell death is apoptosis, necrosis, necroptosis, or a combination thereof.

As used herein, “apoptosis” refers to a form of programmed cell death that occurs in multicellular organisms. Biochemical events including blebbing, cell shrinkage, nuclear fragmentation, chromatin condensation, chromosomal DNA fragmentation, and global mRNA decay lead to characteristic cell changes, such as changes in morphology, and death. In contrast to necrosis, which is a form of traumatic cell death, apoptosis is a highly regulated and controlled process that confers advantages during an organism's life cycle. Unlike necrosis, apoptosis produces cell fragments called apoptotic bodies that phagocytic cells are able to engulf and remove before the contents of the cell can spill out onto surrounding cells and cause damage to them.

“Necrosis” is a form of cell injury which results in the premature death of cells in living tissue by autolysis. Necrosis is caused by external factors, such as infection, or trauma which result in the unregulated digestion of cell components. While apoptosis often provides beneficial effects to the organism, necrosis is almost always detrimental and can be fatal. Cellular death due to necrosis does not follow the apoptotic signal transduction pathway, but rather relies on the activation of various receptors which results in the loss of cell membrane integrity and an uncontrolled release of products of cell death into the extracellular space. This initiates an inflammatory response in the surrounding tissue, which attracts leukocytes and nearby phagocytes which eliminate the dead cells by phagocytosis. Microbial damaging substances released by leukocytes can create collateral damage to surrounding tissues, which can also inhibit the healing process.

“Necroptosis” is a programmed form of necrosis, or inflammatory cell death. Cells can execute necrosis in a programmed fashion making apoptosis not the always preferred form of cell death. The immunogenic nature of necroptosis favors its participation in certain circumstances, such as aiding in defense against pathogens by the immune system. Necroptosis is well defined as a viral defense mechanism, allowing the cell to undergo “cellular suicide” in a caspase-independent fashion in the presence of viral caspase inhibitors to restrict virus replication. In addition to being a response to disease, necroptosis has also been characterized as a component of inflammatory diseases such as Crohn's disease, pancreatitis, and myocardial infarction.

By binding to AIF and/or to PPIA, the peptides described herein disrupts PPIA/AIF complex formation and/or activity. In turn, disrupting PPIA/AIF complex formation and/or activity can inhibit AIF binding to PPIA; inhibit AIF cleavage, oxidation, and translocation to the nucleus of myocytes; reduce HMGB1 nuclear export; reduce DNA fragmentation; reduce cell death by necrosis, necroptosis and/or apoptosis, or a combination thereof.

In many aspects, by binding to AIF and/or to PPIA, the peptides described herein disrupt the PPIA/AIF complex formation and/or activity and, in turn, inhibit myocardial cell death.

In another embodiment, the invention provides an isolated nucleic acid sequence encoding a peptide described herein.

In an additional embodiment, the invention provides a pharmaceutical composition including an isolated peptide described herein and a pharmaceutically acceptable carrier.

As used herein, “pharmaceutical composition” refers to a formulation including an active ingredient and optionally a pharmaceutically acceptable carrier, diluent, or excipient. The term “active ingredient” can interchangeably refer to an “effective ingredient” and is meant to refer to any agent that is capable of inducing a sought-after effect upon administration. In one embodiment, the active ingredient includes a biologically active molecule. As used herein, the phrase “biologically active molecule” refers to a molecule that has a biological effect on a cell. In certain embodiments, the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, a siRNA-peptide conjugate, and siRNA-antibody conjugate, an antagomir, a PNA (peptide nucleic acid), an LNA (locked nucleic acids), or a morpholino. In certain illustrative embodiments, the active agent is a polypeptide or peptide with an amino acid sequence set forth in SEQ ID NO:1-60.

By “pharmaceutically acceptable,” it is meant the carrier, diluent, or excipient must be compatible with the other ingredients of the formulation and not deleterious to the recipient thereof, nor to the activity of the active ingredient of the formulation. Pharmaceutically acceptable carriers, excipients, or stabilizers are well known in the art, for example, Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

In one aspect, the pharmaceutically acceptable carrier is selected from the group consisting of phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; sucrose; mannitol; trehalose; sorbitol; sodium; saline; metal surfactants; non-ionic surfactants; polyethylene glycol (PEG); magnesium stearate; water; alcohol; saline solution; glycol; mineral oil and dimethyl sulfoxide (DMSO).

The pharmaceutical composition of the present invention can be prepared, for example, in liquid form, or can be in dried powder form (e.g., lyophilized). Particular methods of administering pharmaceutical compositions are described below.

In one embodiment, the invention provides a method of preventing myocardial cell death and/or sudden cardiac death in a subject including administering to a subject in need thereof a therapeutically effective amount of the pharmaceutical composition described herein, thereby preventing myocardial cell death and/or sudden cardiac death.

Sudden cardiac death is typically defined as natural, unexpected death from cardiac arrest within one hour of the onset of collapse symptoms, excluding additional time on mechanical life support. Most causes relate to congenital or acquired cardiovascular disease with no symptoms noted before the fatal event. The single most important predictor is fainting or near fainting during exercise, which should require detailed explanation and investigation, as it may reflect the loss of myocardial cell (or myocardial cell death). Sudden cardiac death can be attributed to several causes, including hypertrophic cardiomyopathy, commotio cordis, coronary artery anomalies, left ventricular hypertrophy of undetermined origin, myocarditis, ruptured aortic aneurysm (Marfan syndrome), arrhythmogenic right ventricular cardiomyopathy (ARVC), arrhythmogenic right ventricular dysplasia (ARVD), arrhythmogenic left ventricular cardiomyopathy (ALVC), arrhythmogenic cardiomyopathy (ACM), tunneled coronary artery, aortic valve stenosis, and atherosclerotic coronary artery disease.

The term “preventing”, as used herein, refers to methods or therapeutic methods that have prophylactic/preventative properties. For example, the therapeutic methods described herein are intended to avoid myocardial cell death in a subject, which can be responsible for sudden cardiac death of the subject.

The term “subject” as used herein refers to any individual or patient to which the methods of the invention are performed. Generally, the subject is human, although as will be appreciated by those in the art, the subject may be an animal. Thus, other animals, including vertebrate such as rodents (including mice, rats, hamsters, and guinea pigs), cats, dogs, rabbits, farm animals including cows, horses, goats, sheep, pigs, chickens, etc., and primates (including monkeys, chimpanzees, orangutans, and gorillas) are included within the definition of subject.

By “therapeutically effective amount”, “effective dose,” “therapeutically effective dose”, “effective amount,” or the like it is meant an amount of the pharmaceutical composition of the invention that will elicit the biological or medical response of a tissue, system, animal or human that is being sought by the researcher, veterinarian, medical doctor or other clinician. Generally, the response is either amelioration of symptoms in a patient or a desired biological outcome (e.g., preventing myocardial cell death and/or preventing sudden cardiac death of the subject). In one aspect, the pharmaceutical composition includes a therapeutically effective amount of an isolated peptide having an amino acid sequence as set forth in any of SEQ ID NOs:1-66.

“Administration of” or “administering” should be understood to mean providing the pharmaceutical composition of the invention in a therapeutically effective amount to the subject in need of treatment. Administration routes can be enteral, topical, or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, oral, sublingual buccal, rectal, vaginal, nasal ocular administrations, as well infusion, inhalation, and nebulization.

In some aspects, the subject has an inherited or acquired cardiomyopathy.

Cardiomyopathy is a group of diseases that affect the heart muscle. Early on, there may be few or no symptoms, but as the disease worsens, shortness of breath, feeling tired, and swelling of the legs may occur due to the onset of heart failure. An irregular heartbeat and fainting may occur, putting those affected at an increased risk of sudden cardiac death. Types of cardiomyopathy include hypertrophic cardiomyopathy, dilated cardiomyopathy, restrictive cardiomyopathy, arrhythmogenic right ventricular dysplasia, and Takotsubo cardiomyopathy (broken heart syndrome). In hypertrophic cardiomyopathy, the heart muscle enlarges and thickens. In dilated cardiomyopathy, the ventricles enlarge and weaken. In restrictive cardiomyopathy the ventricle stiffens. In many cases, the cause cannot be determined. Hypertrophic cardiomyopathy is usually inherited, whereas dilated cardiomyopathy is inherited in about one-third of cases. Dilated cardiomyopathy may also result from alcohol, heavy metals, coronary artery disease, cocaine use, and viral infections. Restrictive cardiomyopathy may be caused by amyloidosis, hemochromatosis, and some cancer treatments. Broken heart syndrome is caused by extreme emotional or physical stress. Treatment depends on the type of cardiomyopathy and the severity of symptoms. Treatments may include lifestyle changes, medications, or surgery. Surgery may include a ventricular assist device or heart transplant.

The methods described herein rely on the administration of an isolated peptide having an amino acid sequence as set forth in any of SEQ ID NOs:1-66. The isolated peptides bind to AEF and/or to PPIA and inhibit Ca²⁺-Calpain-1 (CAPN1)-induced cell death (by apoptosis, necrosis, and/or necroptosis) in myocytes. By disrupting PPIA/AIF complex formation and/or activity, the peptides described herein inhibit AIF binding to PPIA; inhibit AIF cleavage, oxidation, and translocation to the nucleus of myocytes; reduce HPMGBI nuclear export; reduce DNA fragmentation and reduce cell death by necrosis, necroptosis and/or apoptosis. The peptides described herein can therefore be used to prevent myocardial cell death and/or sudden cardiac death in a subject.

By “preventing myocardial cell death” and “preventing sudden cardiac death of a subject”, it is meant that the peptides described herein can be used to prevent such event that may occur in all forms of cardiomyopathy, inherited or acquired, triggered by exercise or any other organismal or environmental stressing conditions, susceptible of inducing myocardial cell death and/or sudden cardiac death in a subject. In many aspects, the inherited or acquired cardiomyopathy is arrhythmogenic cardiomyopathy (ACM).

Arrhythmogenic cardiomyopathy (ACM), arrhythmogenic right ventricular dysplasia (ARVD), arrhythmogenic right ventricular cardiomyopathy (ARVC), or arrhythmogenic left ventricular cardiomyopathy (ALVC) is an inherited heart disease. ACM is caused by genetic defects in desmosomes that affect the myocardium or cardiac muscle known. Desmosomes are areas on the surface of heart muscle cells that link the cells together. They are composed of several proteins, and many of those proteins can have harmful mutations. ACM is a non-ischemic cardiomyopathy that primarily involves the right ventricle, though cases of exclusive left ventricular and biventricular disease have been reported. It is characterized by dyskinetic and/or hypokinetic areas involving the free wall of the left, right or both ventricles, with myocardial inflammation, fibrofatty replacement of the myocardium, with associated arrhythmias often originating in the right ventricle. ACM is an important cause of ventricular arrhythmias in children and young adults. It is seen predominantly in males, and 30-50% of cases have a familial distribution.

In one aspect, the ACM is caused by a mutation in desmosomal gene. In various aspects, the mutation is a pathogenic variant in a PKP2, DSG2, DSC2, DSP, or JUP.

Plakophilin-2 is a protein that in humans is encoded by the PKP2 gene. Plakophilin-2 is expressed in skin and cardiac muscle, where it functions to link cadherins to intermediate filaments in the cytoskeleton. In cardiac muscle, plakophilin-2 is found in desmosome structures located within intercalated discs. Mutations in PKP2 have been shown to be causal in arrhythmogenic right ventricular cardiomyopathy. The desmosomal protein, desmoplakin (DSP), is the core constituent of the plaque which anchors intermediate filaments to the sarcolemma by its C-terminus and indirectly to sarcolemmal cadherins by its N-terminus, facilitated by plakoglobin (JUP) and plakophilin-2. Plakophilin-2 is necessary for normal localization and desmoplakin content to desmosomes, which may be due to the recruitment of protein kinase C alpha to desmoplakin. Mutations in PKP2 have been associated with, have been shown to cause, and are considered common in arrhythmogenic right ventricular cardiomyopathy, which is characterized by fibrofatty replacement of cardiomyocytes, myocardial inflammation, ventricular dysfunction and tachycardia, and sudden cardiac death. Mechanistic studies have shown that certain PKP2 mutations result in instability of the plakophilin-2 protein due to enhanced calpain-mediated degradation.

Desmoglein-2 is a protein that in humans is encoded by the DSG2 gene. Desmoglein-2 is highly expressed in epithelial cells and cardiomyocytes and is localized to desmosome structures at regions of cell-cell contact and functions to structurally adhere adjacent cells together. Mutations in desmoglein-2 have been associated with arrhythmogenic right ventricular cardiomyopathy and familial dilated cardiomyopathy. Mutations in DSG2 have been identified in patients with arrhythmogenic right ventricular cardiomyopathy, along with other desmosomal proteins PKP2 and DSP. Ultrastructural analysis has identified the presence of intercalated disc remodeling in these patients. Additionally, the Val55Met mutation in DSG2 was identified as a novel risk variant for familial dilated cardiomyopathy; patients carrying this mutation exhibited shortened desmosomal structures at cardiac intercalated discs compared to non-diseased patients.

Desmocollin-2 is a protein that in humans is encoded by the DSC2 gene. Desmocollin-2 is a cadherin-type protein that functions to link adjacent cells together in desmosomes. Desmocollin-2 is widely expressed and is the only desmocollin isoform expressed in cardiac muscle, where it localizes to intercalated discs. Mutations in DSC2 have been causally linked to arrhythmogenic right ventricular cardiomyopathy.

In various aspects, the method further includes administering to the subject an additional therapeutic treatment.

The administration of the pharmaceutical composition may be by single or multiple doses, alone or in combination with additional therapy. A physician for each particular patient may optimize the amount of isolated peptide and the frequency of dosing. In one aspect, the pharmaceutical composition is administered in a single dose, daily.

The administration of the pharmaceutical composition of the invention can be in combination with one or more additional therapeutic treatments. The phrases “combination therapy”, “combined with” and the like refer to the use of more than one medication or treatment simultaneously to increase the response. The composition of the present invention might for example be used in combination with other treatments to treat or prevent ACM. Specifically, the administration of the peptide of the present invention to a subject can be in combination with a beta-blocker, an antiarrhythmic agent, an anticoagulant, or an implantable cardioverter-defibrillator.

In some aspects, the additional therapeutic treatment is administered prior to, simultaneously with, or after the administration of the pharmaceutical composition of the present invention.

Presented below are examples discussing AIF and PPIA binding peptides contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### MATERIAL AND METHODS

Swim Protocol

Mice underwent two weeks of swim training beginning at 5 weeks of age, where mice swam 10 minutes on Training Day 1 (Monday) then each following day 10-minute increments would be added. Whereby the end of Training Day 10 (second Friday), mice swam 90 mins/day, 5 days/week until swim endpoint (16 weeks of age). No mouse swam on Saturday or Sunday.

Cardiac Function (Echocardiography and Electrocardiography)

All exercised, and age-matched sedentary mice were assessed for cardiac function via transthoracic echocardiography and electrocardiography (ECG) telemetry. Transthoracic echocardiography was assessed in non-sedated mice using a Vevo 2100 Visualsonic imaging system, and measurements were obtained according to the American Society of Echocardiography. Parasternal long-axis view of the left ventricle (LV), at the level of the papillary muscles, were acquired at a sweep speed of 200 mm/sec (M-mode). Three to five measurements were acquired from each mouse and averaged.

ECG Telemetry was obtained. In brief, mice underwent nose cone anesthesia (2% isoflurane, in 100% O₂), and an ECG wireless telemeter (model ETA-F10, DSI PhysioTel) was implanted (subcutaneous, ventral) and sutured in place (7-0 Ethicon) following manufacture's protocol (DSI PhysioTel) to obtain Lead I ECG recordings. All sedentary and exercised mice became fully ambulatory within 2-3 mins following implantation and were given 3 days for recovery. Following implantation recovery, ECGs were recorded for either 24 hours in sedentary cohorts or for 90 minutes in exercised cohorts during a 90-minute swim. All ECG parameters were analyzed utilizing the LabChart Pro ECG Analysis Add-on Software (LabChart Pro 8, MLS360/8, ADIntruments). Following functional data collection (echocardiography and ECG telemetry), all exercised mice and sedentary age-matched cohorts (16 weeks of age) were euthanized and hearts excised for the following experimental protocols listed below.

Electron Paramagnetic Resonance

Stock solutions of 1-hydroxy-3-methoxycarbonyl-2,2,5,5-tetramethylpyrrolidine hydrochloride (CMH; Enzo Life Sciences, Farmingdale, NY) were prepared daily in nitrogen purged 0.9% (w/v) NaCl, 25 μg/L Chelex 100 (Bio-Rad) and 0.1 mM diethylenetriaminepentaacetic acid (DTPA), and kept on ice. Left and right ventricular tissue were homogenized in phosphate-buffered saline (PBS) containing a 0.1 mM DTPA and protease inhibitor cocktail (Roche Applied Science, Indianapolis, IN) pH 7.4. Insoluble fractions were removed by centrifugation at 15,000 g for 10 min (4° C.). Homogenates were kept on ice and analyzed immediately. Samples were treated with 1 mM CMH at 37° C. for 2 min, transferred to 0.05 ml glass capillary tubes, and analyzed on a Bruker E-Scan (Billerica, MA) electron paramagnetic resonance (EPR) spectrometer. EPR spectrometer settings were as follows: sweep width, 100 G; microwave frequency, 9.75 GHz; modulation amplitude, 1 G; conversion time, 5.12 ms; receiver gain, 2×10³; the number of scans, 16. EPR signal intensities were normalized with respect to tissue homogenate protein concentrations as determined by the Pierce BCA protein assay kit (Life Technologies).

Mouse ES-CM Cell Differentiation and Treatment

Mouse embryonic stem cells (ESs) were maintained and differentiated, and the absence of mycoplasma contamination was confirmed prior to experimental studies. Briefly, mouse ESs from both WT and Dsg2^{mut/mut} mice were derived from E3.5 embryos and maintained on gelatin-coated dishes in maintenance medium (Glasgow minimum essential medium supplemented with 10% fetal bovine serum and 3 μM Chir99021 and 1 μM PD98059 or 1000 U/ml ESGRO; Millipore), Glutamax, sodium pyruvate, and MEM non-essential amino acids (Thermo Fisher Scientific). For cardiomyocyte (CM) differentiation, ESs were plated in IMDM/Ham's F12 (Cellgro, at 3:1) supplemented with N2, B27, penicillin/streptomycin, 2 mM GlutaMAX, 0.05% BSA, 5 ng/ml L-ascorbic acid (Sigma-Aldrich), and α-monothioglycerol (MTG; Sigma-Aldrich) at a final density of 100,000 cells/ml to allow spheroid formation. After 48 hours, embryoid bodies were collected and transferred to ultra-low attachment plastic surface and induced for 40 hours with Activin A and Bmp4 (R&D Systems). Cells were then dissociated and plated as monolayers for 48 hours in the presence of 10 μM XAV to allow cardiomyocyte differentiation. The resulting ES-CMs were cultured 45 days before experimental procedures were performed.

Four ES-CM experimental protocols were performed.

(1) Acute (1 day) vs Chronic (7 days) β-adrenergic and Calcium Overload Experiments: WT and Dsg2^{mut/mut} ES-CMs were treated for 1 or 7 days with media partially (50%) changed every day containing either (a) control media; (b) media containing 50 M isoproterenol (ISO) alone; or (c) media containing 50 μM ISO and 1 μM Ca²⁺.

(2) Chronic Calpain-1 (CAPN1) Inhibition Experiments: WT and Dsg2^{mut/mut} ES-CM media were incubated with 30 μM calpeptin (CAPN1 inhibitor, Cat. No. 0448, Tocris) for 2 hours prior to the addition of 50 μM ISO/1μ M Ca²⁺ on day 1 and each consecutive day for 7 days; where 30 μM calpeptin was re-administered each following morning for 2 hrs prior to partial (50%) 50 μM ISO/1 μM Ca²⁺ media changed.

(3) Chronic AIF-TAT Mimetic Peptide Inhibition Experiments: WT and Dsg2^{mut/mut} ES-CM media were incubated with either 0 μM, 5 μM or 25 μM AIF-TAT mimetic peptide (provided by IBB-CNR, Naples, Italy) for 2 hours prior to the addition of 50 μM ISO/1 μM Ca²⁺ on day 1 and each consecutive day for 7 days; where 0 μM, 5 μM or 25 μM AIF-TAT mimetic peptide was re-administered every other morning for 2 hrs before partial (50%) 50 μM ISO/1 μM Ca²⁺ media changed.

(4) After 7 days of treatment, ES-CMs were stained with AnnexinV in a buffer containing 10 mM HEPES (pH 7.4), 140 mM NaCl, 2.5 mM CaCl₂, followed by the addition of 1 mg/ml propidium iodide and analyzed by flow cytometry.

HL-1 Cell Culture and Treatment

Cell Culture: HL-1 cardiomyocytes, an immortalized cell line derived from mouse atria cardiac myocytes, which maintains a differentiated adult cardiac phenotype. Cells were cultured as a monolayer (37° C., 5% CO₂) in plates pre-coated with 1 μg/cm² fibronectin and 0.02% (w/v) gelatin solution. HL-1 cells were maintained in the Claycomb medium (Sigma) supplemented with 10% fetal bovine serum (FBS), 100 U/mL penicillin, 0.1 mg/mL streptomycin, 0.1 mM norepinephrine, and 4 mM L-glutamine. Each experiment was performed 48 h after cell plating.

Calcium (Ca²⁺) Overload Protocol: Rapid and sustained intracellular Ca²⁺ overload was obtained by adding the Ca²⁺-ionophore, calcimycin (1 μM), to HL-1 cells incubated for 10 min in HBS KV medium (1.26 mM CaCl₂, 0.81 mM MgSO₄, 0.44 mM KH₂PO₄, 142 mM KCl, 0.27 mM K₂HPO₄, 5.55 mM D-glucose, 1 mM Na₃VO₄, 20 mM Hepes pH 7.4). To monitor intracellular Ca²⁺ variations, HL-1 cells were loaded with 5 μM Fluo-4 FF AM (Molecular Probes) in HBS Na (Sigma) for 30 min before the media was replaced with HBS KV. The fluorescence was recorded using a fluorimeter plate reader (Fluoroskan Ascent Labsystem) at wavelength settings of 485/530 nm for excitation/emission, respectively.

Calpain (CAPN) Activity Assay: CAPN activity was measured as proteolytic cleavage of the fluorescent CAPN substrate, Suc-LLVY-AMC (SEQ ID NO: 81) (Suc-Leu-Leu-Val-Tyr-7-Amino-4-Methyl-Coumarin (SEQ ID NO: 81), Calbiochem). HL-1 cells were grown in 24-well plates and incubated for 30 min in HBS Na (Sigma) with 25 μM Suc-LLVY-AMC (SEQ ID NO: 81) and 0.01% pluronic F127 (Molecular Probes) before treatment protocols. The fluorescence was recorded using a fluorimeter plate reader (Fluoroskan Ascent Labsystem) at wavelength settings of 390/460 nm for excitation/emission, respectively. CAPN activity was inhibited via the addition of either calpeptin (Tocris) or PD150606 (Calbiochem) in the incubation medium.

Cell Viability Assay: HL-1 plasma membrane integrity was assessed by measuring lactate dehydrogenase (LDH) release in the culture supernatant. An enzymatic assay evaluated LDH activity. Samples of the cell-free culture supernatant were collected at indicated times in Figures/Figure Legends, while cells were lysed in 1% (w/v) Triton X-100 at the end of each experiment to estimate the remaining cellular content of LDH. The LDH activity at each time point was expressed as a percentage of total LDH activity.

Mitochondrial Membrane Potential (MMP) Assay: MMP was determined using the potentiometric probe, JC-1. JC-1 (Molecular Probes) is a cationic carbocyanine dye that selectively accumulates in polarized mitochondria, where it forms red fluorescent aggregates (excitation 485/emission 590 nm). Upon mitochondrial depolarization, JC-1 is released into the cytosol, whereas a monomer emits green fluorescence (excitation 485 nm/emission 520 nm). Therefore, MMP variations were monitored as the ratio of fluorescent emissions at 590/520 nm using a fluorimeter plate reader (Fluoroskan Ascent Labsystem). HL-1 cells were incubated with JC-1 (1.5 μM) for 30 min at 37° C. in HBS Na before treatment protocols.

Calpastatin-overexpression: For cell line transfection, both coverslips and cell culture plate surfaces were pre-treated with the following coating solutions: 100-200 μl of Opti-MEM reagent was combined with CAST-overexpression 0.66 μg/cm² plasmid containing a GFP-reporter and allowed to sit at room temperature for 5 min. Then, 100-200 μl of Opti-MEM reagent was combined with 0.22 l/cm² of cationic lipid reagent and allowed to sit at room temperature for 5 min. These two solutions were then integrated and complexed for 20 min at room temperature. The transfection solution was distributed over the cell seeding surface and left to dry at room temperature for 30-45 min. Cells were counted and resuspended in 2 ml of serum and antibiotic-free medium, then directly spread over the surface. Cells were incubated at 37° C. with 95% O₂ and 5% CO₂ for 30 min, then added to the appropriate serum and antibiotics concentration. Treatments were conducted 24 h after cell transfection. Following transfection and treatment, cell cultures were subjected to calcium overload in the presence of HBS KV medium containing 0.4% Trypan Blue. Live-imaging, fluorescence-based cell viability was detected at 30 min and 60 mins post Ca²⁺ overload.

Western Immunoblotting

Ventricular myocardia, HL-1 cells, and mouse ESC-CMs were lysed in RIPA buffer containing 1:100 phosphatase and proteinase inhibitor cocktails (Sigma-Aldrich) and underwent centrifugation protocols described below to isolate soluble, insoluble, and mitochondrial/nuclear proteins. Protein lysates were quantified via a standard Pierce BCA protein assay kit (Life Technologies). Forty micrograms of protein lysates were separated on either 4-12% or 12% BisTris gels (NuPage, Invitrogen) under non-denaturing conditions with 1X MOPS Running Buffer (Invitrogen). Following transfer to nitrocellulose membranes and blocking (1 hour in 1×PBS containing 5% nonfat milk and 0.1% Tween-20) at room temperature, immunoblots were probed with primary antibodies overnight at 4° C. in blocking buffer. Primary antibodies, corresponding product numbers and concentrations used were as follows: Rabbit monoclonal against AIF (Cell Signaling, 5318S, at 1:3,000); rabbit monoclonal against GAPDH (Cell Signaling, 5174, at 1:10,000); rabbit monoclonal against TXN2 (Cell Signaling, 14907S, at 1:1,000); mouse monoclonal against TXNRD2 (Cell Signaling, 12029S, at 1:1,000); rabbit monoclonal against TXN1 (Cell Signaling, 2429S, at 1:1,000); rabbit monoclonal against TXNRD1 (Cell Signaling, 15140S, at 1:1,000); rabbit monoclonal against cytC (Cell Signaling, 4280S, at 1:3,000); rabbit polyclonal against PRDX3 (Thermofisher, LF-PA0030, at 1:3,000); rabbit polyclonal against CAPN1 (Cell Signaling, 2556S, at 1:1000); mouse monoclonal against CAPN1 Domain III (ThermoFisher, MA3-940, at 1:1000); rabbit polyclonal against CAPN2 (Cell Signaling, 2539S, at 1:1,000), rabbit monoclonal against Histone-3 (Cell Signaling, 4499S, at 1:3,000), rabbit polyclonal against 3-actin (Abcam, ab8227, at 1:5,000), rabbit polyclonal against PARP-1 (BioMol, P3113-20F, at 1:1,000), rabbit polyclonal against caspase-3 (Cell Signaling, 9662, at 1:1,000), mouse monoclonal against OPA1 (BD Transduction Laboratories, 612606, at 1:1,000), mouse monoclonal against Calnexin (BD Transduction Laboratories, 610524, at 1:10,000), mouse monoclonal against plasma membrane Na+/K+-ATPase (Abcam, ab76020, at 1:1,000), rat monoclonal against Lamp2 (Millipore, 428019, at 1:500); mouse monoclonal against MAO-A (Santa Cruz, sc-271123, at 1:1,000); and rabbit polyclonal against COXIV (ThermoFisher, 11242-1-AP, at 1:5,000). The following day immunoblots were washed three times (1X PBS) and probed with species-specific IRDye secondary antibodies (Li-Cor TRDye 800CW or TRDye 680RD at 1:10,000; or HRP-conjugate at 1:10,000) for 1 hour at room temperature in blocking buffer. Immunoblots were then washed three times, and immunoblot images were obtained utilizing the LI-COR Odyssey imaging system.

Mitochondria Isolation and Subcellular Fractionation

Mitochondria Isolation Protocol: Mitochondria were isolated from HL-1 cells in percoll density gradient. Briefly, 30×10⁶ adherent cells were harvested with Trypsin/EDTA, centrifuged at 500×g for 10 min, washed in buffer A (100 mM sucrose, 1 mM EGTA, 20 mM MOPS, pH 7.4, and 1 mg/mL BSA) before cell disruption with a Dounce homogenizer. The suspension was centrifuged twice at 500× g for 5 min and the resulting supernatant at 10,000×g for 10 min at 4° C. The pellet was re-suspended in buffer B (300 mM sucrose, 1 mM EGTA, 20 mM MOPS pH 7.4, 1 mg/mL BSA), and the homogenate was layered on a two-phase (25% and 40%) percoll density gradient. After centrifugation at 30,000×g for 15 min at 4° C., mitochondria (layered at the interface) were removed, washed with buffer A and resuspended in RIPA buffer.

Subcellular Fractionation Protocol: Subcellular fractionation was performed in mouse ventricular myocardium lysed via compartment-specific isolation buffers according to the manufacturer's protocol (Subcellular Fractionation Kit for Tissues, ThermoFisher, 87790). Centrifugation speeds, spin times, temperatures, and resulting subcellular compartment (supernatant) were acquired in sequential order and are as follows:
(i) 500×g, 5 mins at 4° C., Cytosolic Extract;
(ii) 3,000×g, 5 mins at 4° C., Mitochondrial Bound Extract;
(iii) 5,000×g, 5 mins at 4° C., Nuclear Extract; and
(iv) 16,000×g, 5 mins at 4° C., Chromatin-bound Extract.

Additionally, insoluble (mitochondrial- and chromatin-bound lysates) and soluble (cytosolic and nuclear lysates) extracts for AIF's oxidative status were compared.

Immunohistochemical/Immunofluorescence Staining

All myocardium (sedentary and exercised mouse cohorts and patient myocardial biopsies) were formalin-fixed and paraffin-embedded (FFPE). Slides were obtained from FFPE blocks (5 μm thick), then were deparaffinized, rehydrated, underwent antigen retrieval, and blocked for 1 hour at room temperature, then incubated with primary antibodies at 4° C. overnight. The following primary antibodies were used: rabbit monoclonal against AIF (Cell Signaling, 5318S, at 1:250), mouse monoclonal against cardiac Troponin-T (cTnT; Thermofisher, MA5-12960, at 1:500), and rabbit polyclonal against HMGB1 (Thermofisher, PA1-16926, at 1:100). Slides were then washed three times with 1×PBS and probed with species-specific Alexa Fluor secondaries or for 1 hour at room temperature (ThermoFisher, donkey anti-mouse Alexa Fluor 488, R37114, at 1:1,000; and donkey anti-rabbit Alexa Fluor 594, R37119, at 1:1,000). Following three additional washes, ProLong Gold Antifade Mountant with DAPI (Thermofisher, 936931) were applied, and slides were coverslipped, and immunoreactive signal was detected via laser scanning microscopy (Zeiss LSM 510 Meta). Additionally, Masson's trichrome, H&E, and COXIV (ThermoFisher, 11242-1-AP, at 1:500) immunoperoxidase sections were immunostained and imaged on an Olympus BX51TF with a DP70 color camera (Olympus).

DNA Retardation Assay

A DNA retardation (fragmentation) assay was performed, in order to examine the effects oxidized—vs reduced-AIF lysates on the migration of double-stranded DNA. Specifically, a 10 μl sample was prepared to contain (i) 5 μg of protein lysate, (ii) 4 μl of a 100 bp DNA ladder (ranging from 100 bp to 1.5 kb; Promega, G2101), (iii) and 1 μl (400 ng/μl) of the wild-type Dsg2 2.2 kb double-stranded DNA fragment generated via polymerase chain reaction (PCR), then incubated at 37° C. for 30 mins. Following incubation, 5 μl of sample mixture was run on a 3% agarose gel at 150V. A 1 kb DNA ladder (New England BioLabs, N3232L) was used as a reference band.

Tissue Microarray (TMA)

A TMA composed of 94 left ventricular tissues was generated consisting of 33 control tissues (non-cardiac deaths), and three failing heart cohorts: 28 DCM tissues, 8 HCM tissues, and 25 HID tissues. All tissue cores were 1.5 mm in diameter, embedded into a single paraffin block, and cut at 5 μm thickness.

AIF-Colocalization Analysis

Following immunostaining, AIF-fluorescence and nuclear fluorescence (DAPI) were detected via laser scanning microscopy (Zeiss LSM 510 Meta) via a sequential fashion to avoid crosstalk between fluorophores. Merged images were obtained using Zeiss LSM software, and approximately 3-10 Regions of Interest (ROIs) were assessed per condition (mouse myocardium and patient myocardial biopsies). Co-localization analyses were determined by the overlap of fluorophore distribution (x-axis) vs. fluorophore intensity (y-axis) between AIF and DAPI at each ROI. All patient immunostained slides received an AIF pathological score ranging from 0 to 4, as follows: Grade 0 (mitochondrial AIF); Grade 1 (mitochondrial and cytosolic AIF, 50:50 odds ratio); Grade 2 (diffuse cytosolic AIF, rare mitochondrial localization); Grade 3 (presence of 2 or 3 AIF-positive nuclei, determined by the overlap of fluorophore intensity and fluorophore distribution); or Grade 4 (presence of ≥4 AIF-positive nuclei, determined by the overlap of fluorophore intensity and fluorophore distribution). AIF-to-DAPI colocalization pathological scores (Grades 0-4) were only performed in the patient myocardium due to the availability of myocardial biopsy size. The level of total myocardial AIF-positive (AIF+) nuclei was performed in mouse myocardium due to tissue availability and presented as percent AIF+ nuclei over total nuclei.

Thioredoxin Reductase Activity

Stock solutions of NADPH (48 mM) and 5,5′-dithio-bis-[2-nitrobenzoic acid](DTNB, 100 mM) were prepared in MilliQ water and DMSO, respectively. Flash-frozen ventricular tissue was homogenized in 0.1 M potassium phosphate buffer containing 0.1 mM DTPA and protease inhibitor cocktail at pH 7.0.

The samples were then subjected to three sequential freeze/thaw cycles between liquid nitrogen and 37° C. water bath. The insoluble fractions were removed by centrifugation at 14,000×g for 2 min at 4° C., and the protein concentrations were quantified by BCA assay (Pierce). Briefly, the homogenates (0.1 mg/ml) were incubated with 0.24 mM NADPH and 3 mM DTNB in pH 7.0 potassium phosphate buffer in the presence or absence of thioredoxin reductase inhibitor, auranofin (100 nM) for 2 hours at room temperature in the dark. The relative absorbance of 2-nitro-5-thiobenzoate anion (TNB2-, ε412=14150 M-1 cm-1) were detected on a SpectraMax microplate reader (Molecular Devices) at baseline and following 2 hour incubation. In all cases, each sample was analyzed in triplicate, and total TXNRD activity was calculated. One unit of TXNRD activity is the amount of enzyme that generates 1.0 μmol of TNB²⁻ per minute at 25° C.

Mass Tag Labeling

Two hundred micrograms of mouse ventricular protein were incubated with either (i) control solution (RIPA alone) or (ii) 1 mM methoxypolyethylene glycol maleimide (mPEG) in RIPA for 2 hrs at room temperature, in the dark, with nutation. Protein pellets were precipitated via sequential methanol/chloroform/DiH₂O addition (400 μl/150 μl/300 μl) then centrifuged (20,000×g for 5 mins at 4° C., in the dark). Supernatant was discarded and protein pellets were washed with 400 μl methanol, centrifuged at 20,000×g for 5 mins at 4° C. in the dark, and supernatant discarded. Protein pellets were re-suspended in 50 μl of loading buffer, heated to 95° C. for 10 mins, and 40 μg of mPEG and non-mPEG treated samples were run on 4-12% or 12% BT gels under non-reducing conditions.

Statistical Analysis

For all mouse and patient ACM findings, data presented as mean±SEM, specific n-values are inset within each figure legend or table. For all HL-1 cell findings, data presented as mean±StDev from six independent experiments/conditions. P<0.05 was deemed statistically significant. As appropriate, associations between continuous dependent variables were tested using Student's paired/unpaired t-test (binary independent variables) or one-way/two-way ANOVA (two or more variables). All statistical analyses performed are additionally included in each figure legend or table. All protocols and statistical analyses adhere to the AHA Journal's implementation for the TOP guidelines.

### Example 2

### Exercise Triggers Extensive Myocyte Necrosis

### In the Hearts of Dsg2^{mut/mut} Mutant Mice

Endurance exercise exacerbates left ventricular (LV) dysfunction in Dsg2^{mut/mut} mice, a robust mammalian ACM model. Myocyte loss is a primary culprit of LV dysfunction, yet right ventricular (RV) dysfunction is more prominent in ACM patients. Therefore, the impact of chronic swimming on both right (RV) and left ventricle (LV) function in Dsg2^{mut/mut} mice, and the extent of myocyte loss contributing to cardiac dysfunction were determined.

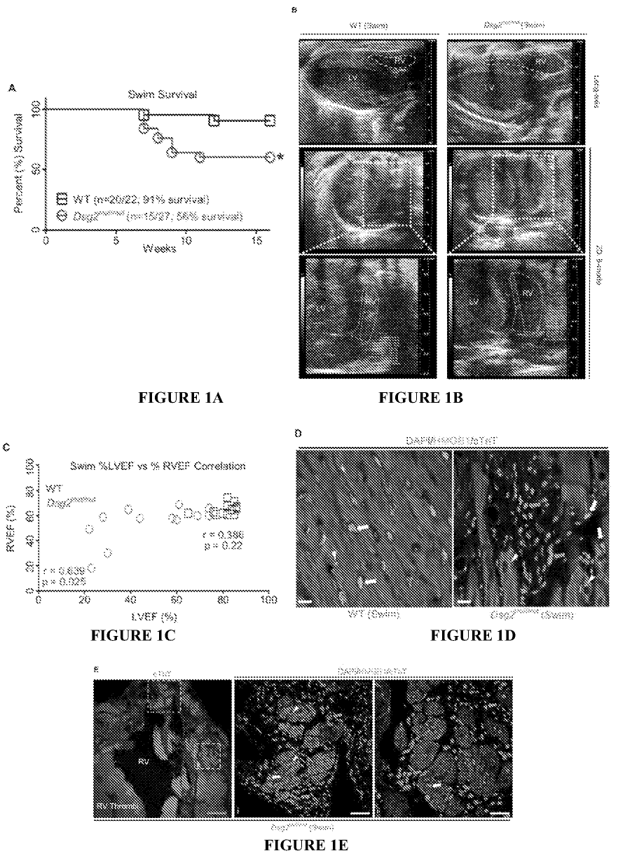

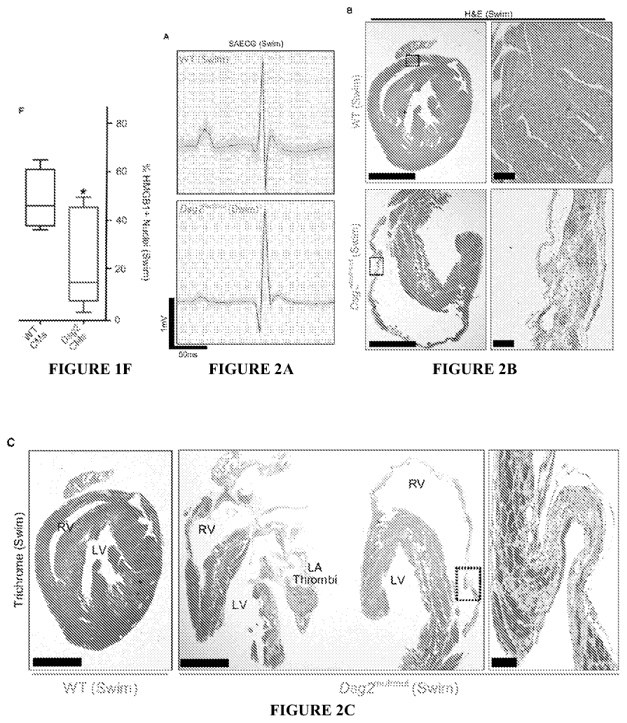

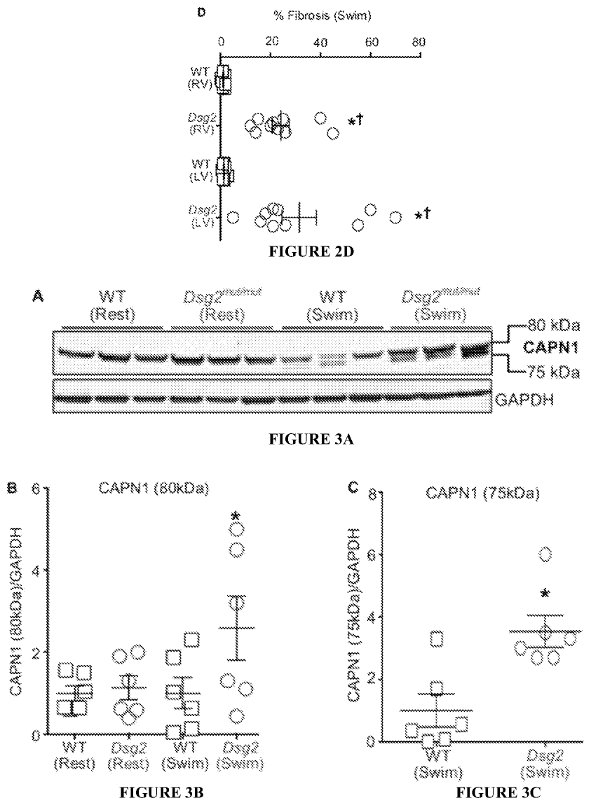

5-week old WT and Dsg2^{mut/mut} mice underwent an 11-week (90 mins/day, 5 days/week) endurance swim protocol. Only 56% (n=15/27) of Dsg2^{mut/mut} mice survived, while almost all WT mice survived (91% survival, n=20/22) to swim endpoint (FIG. 1A). Of the survivors, Dsg2^{mut/mut} mice presented with both LV and RV dysfunction (FIGS. 1B, 2C and Table 5), such as the dilated right ventricle in Dsg2^{mut/mut} mice (RV; dashed area, top panel FIG. 1B) or diffuse epicardial-to-endocardial fibrosis throughout the RV, with highly localized epicardial-to-endocardial fibrotic lesions within the LV free wall from exercised Dsg2^{mut/mut} mice, and aberrant ECG morphologies indicative of repolarization and depolarization abnormalities (Table 5; FIG. 2A), such as reduced S-amplitude and increased Q-amplitude from exercised Dsg2^{mut/mut} mice compared to WT mice. Furthermore, a robust correlative relationship between reduced LV ejection fraction (LVEF) and reduced RVEF was apparent in exercised Dsg2^{mut/mut} mice (FIG. 1C).

TABLE 5
Echocardiographic and electrocardiogram indices
from exercised WT and Dsg2^{mut/mut} mice.

| Parameter | WT | Dsg2^{mut/mut} |
|---|---|---|
| Echocardiography | | |
| N | 20 | 15 |
| IVSd (mm) | 0.97 ± 0.02 | 0.78 ± 0.04* |
| IVSs (mm) | 1.49 ± 0.04 | 1.08 ± 0.06* |
| LVIDd (mm) | 2.80 ± 0.05 | 3.31 ± 0.19* |
| LVIDs (mm) | 1.19 ± 0.06 | 2.24 ± 0.24* |
| RVIDd (mm) | 1.16 ± 0.22 | 1.52 ± 0.52 |
| RVIDs (mm) | 0.42 ± 0.09 | 0.72 ± 0.28 |
| LV-PWTd (mm) | 0.94 ± 0.02 | 0.87 ± 0.03* |
| RV-PWTd (mm) | 0.31 ± 0.03 | 0.32 ± 0.03 |
| RV-EF (%) | 65.5 ± 1.32 | 54.9 ± 4.25* |
| LV-EF (%) | 81.6 ± 1.65 | 56.4 ± 4.79* |
| Electrocardiography | | |
| N | 13 | 13 |
| PR-I (ms) | 43.2 ± 2.1 | 45.7 ± 3.7 |
| Pd (ms) | 11.6 ± 0.7 | 10.8 ± 1.3 |
| QRSd (ms) | 16.7 ± 0.7 | 19.7 ± 1.1 |
| P-Amp (mV) | 0.10 ± 0.01 | 0.036 ± 0.01* |
| R-Amp (mV) | 1.77 ± 0.13 | 1.49 ± 0.14 |
| Q-Amp (mV) | −0.0003 ± 0.002 | −0.12 ± 0.02* |
| S-Amp (mV) | −0.59 ± 0.07 | −0.20 ± 0.03* |
| NSVT (Count) | 4.85 ± 1.3 | 21.1 ± 6.3* |

IVS, interventricular septal end diastole (d) and end systole (s); LVID, left ventricular internal diameter; RVID, right ventricular internal diameter; LV-PWTd, LV posterior wall thickness; RV-PWTd, RV posterior wall thickness; PR-I, PR-Interval; Pd, P duration; QRSd, QRS duration; P-Amp, P-Amplitude; R-Amp, R-Amplitude; Q-Amp, Q-Amplitude; S-Amp, S-Amplitude.
NSVT, non-sustained ventricular tachycardia.
Data presented as mean ± SEM, n-values inset, with a
*P < 0.05 vs. WT using 1-way ANOVA.

Determining the extent and modality (i.e., apoptosis vs. necrosis) by which exercise triggers myocyte cell death in Dsg2^{mut/mut} myocardium is of pathological relevance considering myocardial inflammation and fibrosis (both prominent in Dsg2^{mut/mut} myocardium, FIGS. 2B, 2D) are more often associated with necrosis—than apoptotic-induced cardiac remodeling. Therefore, myocardium from exercised mice was first examined for the commonly used apoptotic labeler, TUNEL, and it was found that exercise indeed increased the number of TUNEL+ nuclei in Dsg2^{mut/mut} myocytes (data not shown). However, in exercised mutants, the extent of myocardial necrosis demonstrably surpassed the number of apoptotic myocytes (FIGS. 1D-1F). Specifically, myocardium was immunolabeled for high mobility group box-1 (HMGB1) to assess global myocardial necrosis. Loss of nuclear HMGB1, a non-histone DNA-binding protein, is a widely used histological index of necrosis.

In hearts from exercised WT mice, HMGB1 was almost exclusively localized in the myocyte nucleus (FIG. 1D), denoting healthy myocytes. In contrast, while Dsg2^{mut/mut} hearts exhibited HMGB1 positive (HMGB1+) nuclei in myocytes, these hearts also exhibited perinuclear- and cytoplasmic-HMGB1 localization, thus indicating active necrosis (FIGS. 1D, 1E). Nuclear HMGB1 release acts as a putative “danger signal,” acting as a chemotactic molecule for professional immune cells to sites of injury. Of note, myocardium from exercised Dsg2^{mut/mut} mice displayed pronounced HMGB1+ nuclei in non-cardiomyocytes (non-CMs) surrounding CMs (FIGS. 1E, 1F). In exercised Dsg2^{mut/mut} hearts, necrotic cells were exclusively myocytes, as infiltrating professional immune cells showed robust HMGB1+ nuclear staining (FIGS. 1E, 1F).

### Example 3

### CALPAIN1 ACTIVATION ACCOUNTS FOR MYOCYTE NECROSIS

### IN EXERCISED Dsg2^{mut/mut} MUTANT MICE

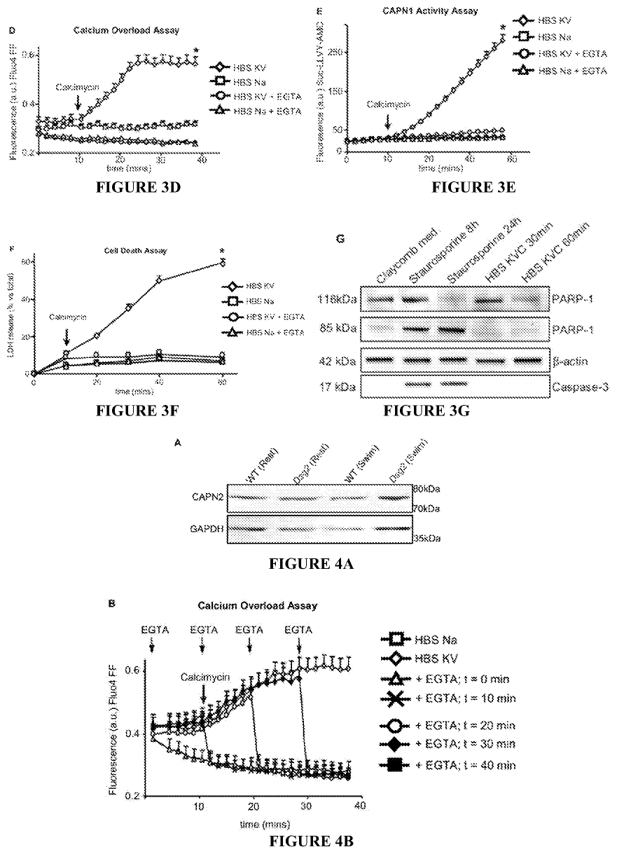

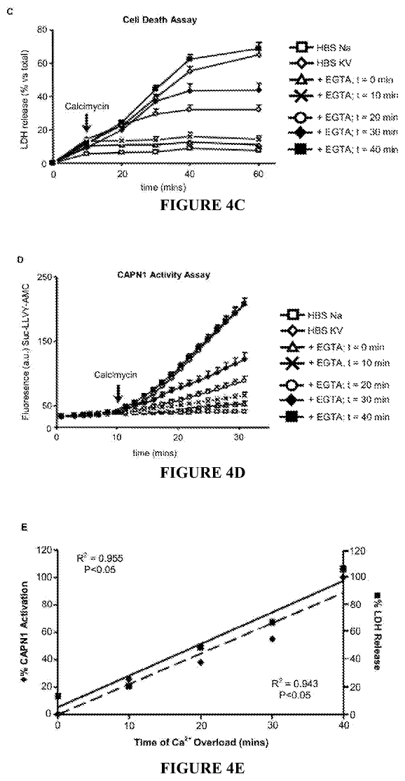

Elevated intracellular calcium (Ca²⁺) is well-documented in human ACM, and abnormal Ca²⁺ handling occurs in isolated Dsg2^{mut/mut} myocytes. Importantly, Ca²⁺ overload is a significant cause of myocardial necrosis, and activation of Ca²⁺-dependent cysteine proteases, calpain-1 (CAPN1) and calpain-2 (CAPN2), are drivers of Ca²⁺ overload-induced necrosis. Therefore, myocardial CAPN1 (FIGS. 3A-3C) and CAPN2 levels were assessed from sedentary and exercised mice (FIG. 4A). In vivo, native 80 kDa CAPNs undergo Ca²⁺-dependent autoproteolytic cleavage generating active 75 kDa CAPN peptides. No changes in native (80 kDa) or active (75 kDa) CAPN2 levels were evident in the myocardium of sedentary and exercised mice, regardless of genotype (FIG. 4A). Conversely, exercised Dsg2^{mut/mut} hearts showed elevated levels of both total, native (80 kDa) CAPN1 and cleaved, active (75 kDa) CAPN1 compared to trained WT hearts (FIGS. 3A-3C).

These in vivo findings led to the investigation of whether increased cytosolic Ca²⁺ levels are necessary and sufficient to activate CAPNs and CAPN-mediated myocyte cell death in vitro. To do so, HL-1 cells (an immortalized cardiac cell line) were used, considering HL-1 cells maintain many features of an adult cardiac phenotype in culture and are used frequently in ACM pathogenesis studies. HL-1 cells were incubated in sodium (Na)-deficient media (HBS) containing potassium (K) to depolarize the plasma membrane and vanadate (V) to inhibit the plasma membrane Ca²⁺-ATPase (HBS KV media). The addition of calcimycin, a divalent cation carrier, in HL-1 cells with HBS KV media resulted in cytosolic Ca²⁺ overload (FIG. 3D). Of note, Ca²⁺-overload occurred in HL-1 cells containing HBS KV media, only. As a control, it was determined that such a rise in Ca²⁺ did not occur upon calcimycin addition in cells incubated in (i) HBS Na medium alone or (ii) in HBS KV and HBS Na medium in the presence of the Ca²⁺-chelating agent, EGTA (FIG. 3D).

To investigate Ca²⁺-mediated CAPN1 activation, cells were preloaded with a synthetic CAPN1 substrate, Suc-LLVY-AMC (SEQ ID NO: 81). CAPN1-mediated substrate cleavage occurred only in cells subjected to Ca²⁺ overload (FIG. 3E). Additionally, Ca²⁺ overload in HL-1 HBS KV treated cells resulted in robust cell death (FIG. 3F). EGTA in the incubation media attenuated both CAPN1 activation and cell death, respectively (FIGS. 3E, 3F). Release of LDH (FIG. 3F) does not discriminate between cell death modality; therefore, whether Ca²⁺ overload-induced cell death occurred via necrosis, apoptosis, or both was investigates. HL-1 cell culture lysates were assessed for changes in known markers of apoptosis, caspase-3, and PARP-1. Consistent with in vivo findings reported in FIG. 1, cell death was primarily due to necrosis, in that cleavage of PARP-1 and caspase-3 was not detected in HL-1 cells subjected to Ca²⁺ overload (lanes 4 and 5; FIG. 3G). Conversely, these bands were evident upon the treatment of HL-1 cells with staurosporin (lanes 2 and 3; FIG. 3G), a well-established inducer of apoptosis. Cells were treated with Calcimycin (C) in HBS KV medium (HBS KVC) or with staurosporin for the indicated times. PARP-1 and caspase-3 proteolysis, indicating the occurrence of apoptosis, was detected as the appearance of bands at 85 kDa and 17 kDa, respectively.

The relationship between the extent of Ca²⁺ overload with CAPN1 activation and cell death was investigated further by adding EGTA to HL-1 cells subjected to Ca²⁺ overload at different times (FIGS. 4B-4E). Calcium deprivation by EGTA caused a considerable decrease in Fluo4 FF fluorescence, permitting a change in the duration of intracellular Ca²⁺ overload; EGTA addition induced a rapid decline of Fluo4 FF fluorescence to the baseline level. (FIG. 4B). Interestingly, EGTA addition to HL-1 cells 10 mins after Calcimycin treatment resulted in minimal cell death. In contrast, the addition of EGTA 40 mins after Calcimycin treatment resulted in the greatest extent of necrosis, even more so than HBS KV media without EGTA (FIG. 4C). Additionally, CAPN1 activation displayed a strict dependence on the duration of Ca²⁺ overload (FIG. 4D). Thus, the time of Ca²⁺ overload exposure directly correlated with both the extent of cell death and CAPN1 activity (FIG. 4E). LDH release (right y-axis) and Suc-LLVY-AMC (SEQ ID NO: 81) (% CAPN1 Activation, left y-axis) hydrolysis were expressed as the percentage of the maximal value obtained after 40 min intracellular Ca²⁺ overload. Pearson's r (R2) correlation analyses and P-values inset.

HL-1 cells were exposed to Calcimycin (1 μM, black arrows) to induce calcium (Ca²⁺) overload and the presence of the Ca²⁺ chelating agent, EGTA (5 mM) at different time points (indicated by the arrows in FIGS. 4A-4E).

### Example 4

### Exercised Dsg2^{Mut/Mut} Hearts Display Prominent Calpastatin Depletion, and Calpastatin Overexpression Offsets Ca²⁺ Overload-Induced Cell Death

CAPN1 activation is endogenously inhibited by calpastatin (CAST), and CAST is also a CAPN1 substrate. Whether any change in endogenous CAST levels and proteolytic CAST degradation product(s) occurs in Dsg2^{mut/mut} mice compared to WT, at rest and in response to swimming was tested.

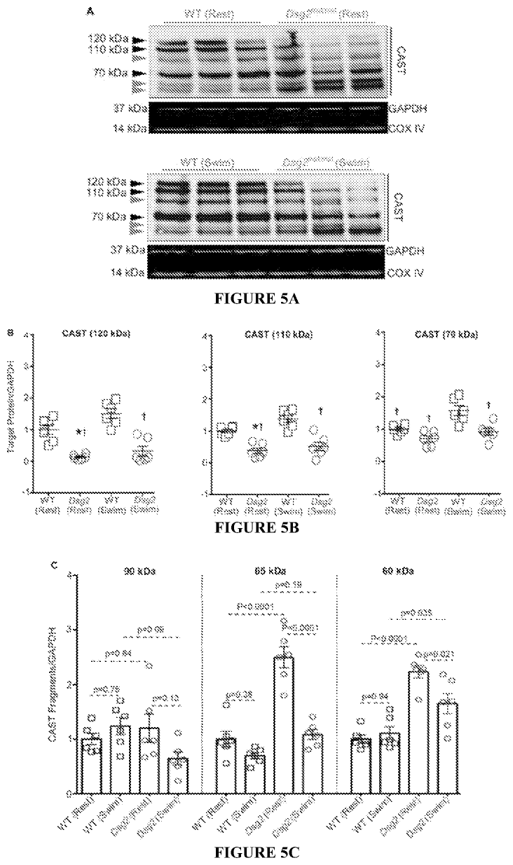

Of note, the highest molecular weight CAST protein detected in cardiac tissue, regardless of genotype or condition, migrated at an expected weight of 120 kDa (FIG. 5A). Additionally, 110 kDa and 70 kDa CAST isoforms were identified. CAST exhibits tissue- and cell-specific isoforms, where skeletal (145/135 kDa doublet), cardiac (120/110 kDa doublet), T-cell (70 kDa isoform), and erythrocyte (70 kDa isoform) CAST isoforms have been identified, all containing CAPN1 binding domains. Compared to WT cohorts,

Dsg2^{mut/mut} mice harbored reduced levels of the 120/110 kDa doublet, either at rest or in response to swimming (FIG. 5B). Interestingly, the 120/110 kDa doublet and 70 kDa CAST isoforms were more evident in exercised WT mice compared to sedentary WT cohorts (FIGS. 5A, 5B). Conversely, only the levels of the 110 kDa and 70 kDa CAST isoforms increased in exercised vs. sedentary Dsg2^{mut/mut} mice (FIGS. 5A, 5B). Elevation in CAST expression in WT and mutant swimmers' hearts may not be an anomaly, but an exercise-induced adaptation. Indeed, prior reports also demonstrated CAST expression is elevated in skeletal and cardiac tissues in response to exercise and/or β-adrenergic stimulation. Next, CAST proteolytic fragments were analyzed. Of note, regardless of genotype or condition, myocardial CAST fragments at 90 kDa and a 65/60 kDa doublet were observed (FIGS. 5A, 5C). Both 65 kDa and 60 kDa CAST fragments were potentiated in sedentary and exercised myocardial lysates from Dsg2^{mut/mut} mice vs. WT counterparts (FIGS. 5A, 5C). Thus, native CAST depletion and increased CAST degradation may account for elevated Ca²⁺/CAPN1-induced necrosis in Dsg2^{mut/mut} mice. The observed increase in 60-65 kDa CAST fragments in ACM mice could result from CAPN1-mediated proteolysis of 70 kDa CAST in circulating myocardial erythrocytes from infiltrating T-cells, which are heavily present in Dsg2^{mut/mut} mouse hearts.

Next, whether overexpressing CAST levels would prevent Ca²⁺-overload, CAPN1-induced cell death observed in FIG. 3 was determined. To this end, the CAPN1-inhibitor domain of CAST was fused with a GFP reporter and this CAST-GFP construct was overexpressed in HL-I cells. The relatively low efficiency of the transfection generated a mixed population of CAST-GFP positive cells and non-transfected (GFP negative) cells. Following transfection, HL-1 cells were cultured in HBS KV medium containing 0.4% Trypan Blue (TB) then subjected to Ca²⁺ overload for 1 hr (FIG. 5D). Trypan blue admits fluorescence via confocal microscopy and is only taken up in dead cells. HL-1 cells overexpressing CAST (green cells) were protected from Ca²⁺ overload-induced cell death (absence of fluorescence in cells). TB emits red fluorescence via confocal microscopy when sequestered by dead/dying cells. Live-imaging, fluorescence-based cell viability was then detected at 30 min and 60 mins post-Ca²⁺ overload. HL-I CAST-GFP positive cells were protected from Ca²⁺ overload-induced cell death (FIG. 5D). In vivo and in vitro data collectively suggest that amplifying CAST levels can prevent Ca²⁺/CAPN1-induced necrosis in myocytes.

### Example 5

### Mitochondrial Dysfunction Precedes Ca²⁺/CAPN1-Mediated Necrosis

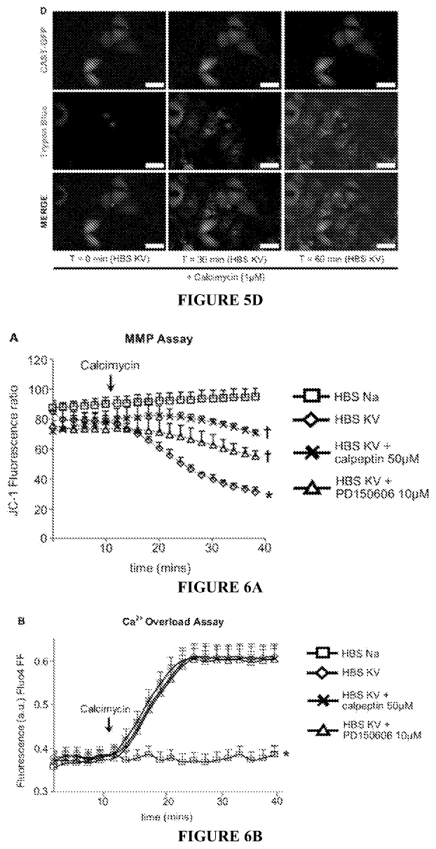

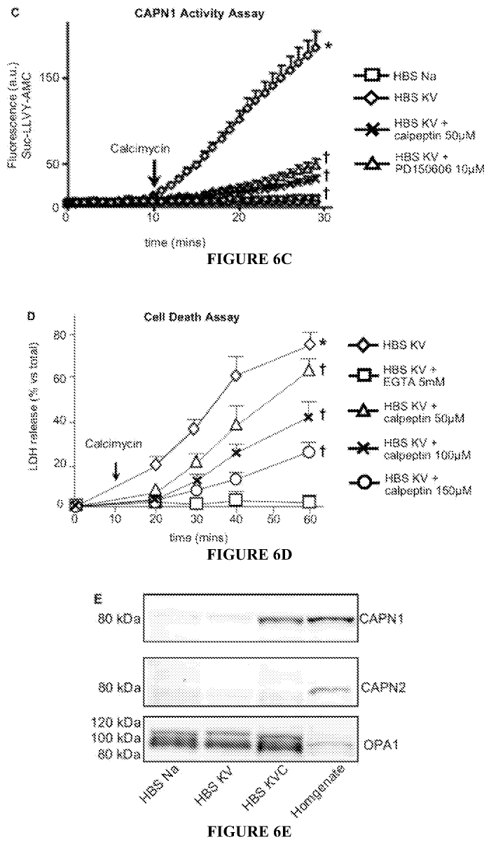

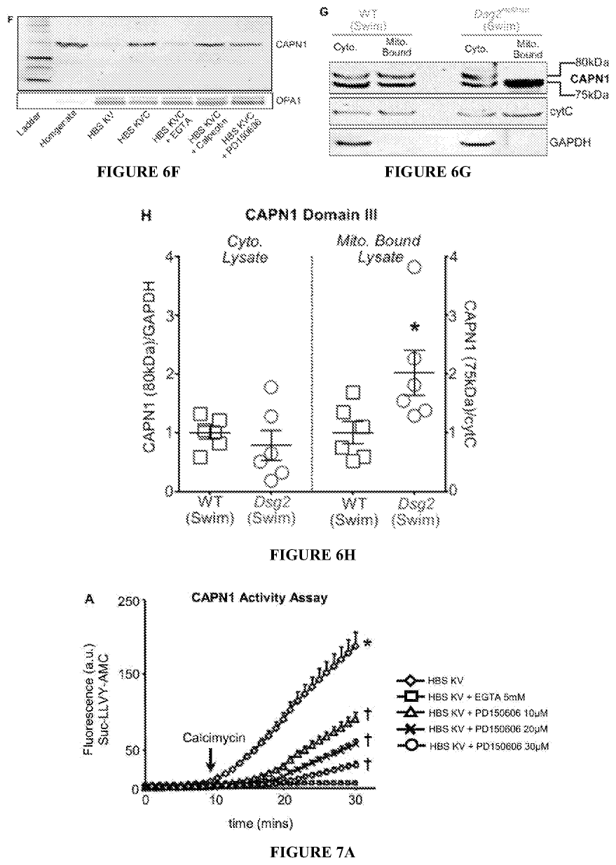

Mitochondrial perturbations are implicated in many cell death modalities, and mitochondrial alterations and intracellular Ca²⁺ overload ([Ca²⁺]i) occur in cardiac disorders, such as ischemia-reperfusion (I/R). Cardiomyocytes derived from human pluripotent stem cells from an ACM patient display mitochondrial dysfunction and concomitant cell death. Hence, mitochondrial membrane potential (MMP) was monitored in HL-I cells challenged with Ca²⁺ overload, in the presence or absence of two different CAPN1 inhibitors (calpeptin and PD150606) (FIG. 6A). Of note, calpeptin interacts with the catalytic site of CAPN1, while PD150606 interferes with the EF-hand domain (Ca²⁺ binding site) of CAPN1. Challenging HL-1 cells with Ca²⁺ overload decreased MMP, and calpeptin significantly attenuated this effect (FIG. 6A). Conversely, HL-1 cells treated with PD150606 showed a reduced ability to restore MMP (FIG. 6A). Interestingly, while both inhibitors attenuated Ca²⁺ overload-induced decrease in MMP (FIG. 6A); neither of them had an impact on the kinetics of cytosolic Ca²⁺ accumulation (FIG. 6B). For FIGS. 6A and 6B, HL-1 cells were subjected to calcium (Ca²⁺) overload induced by Calcimycin addition (1 μM, black arrows) in HBS Na medium, and HBS KV medium in the absence or presence of the calpain inhibitors, calpeptin or PD150606. HL-1 cells were subjected to Ca²⁺ overload (via Calcimycin, black arrows) in HBS KV medium in the absence or presence of a calpeptin dose-dependent response.

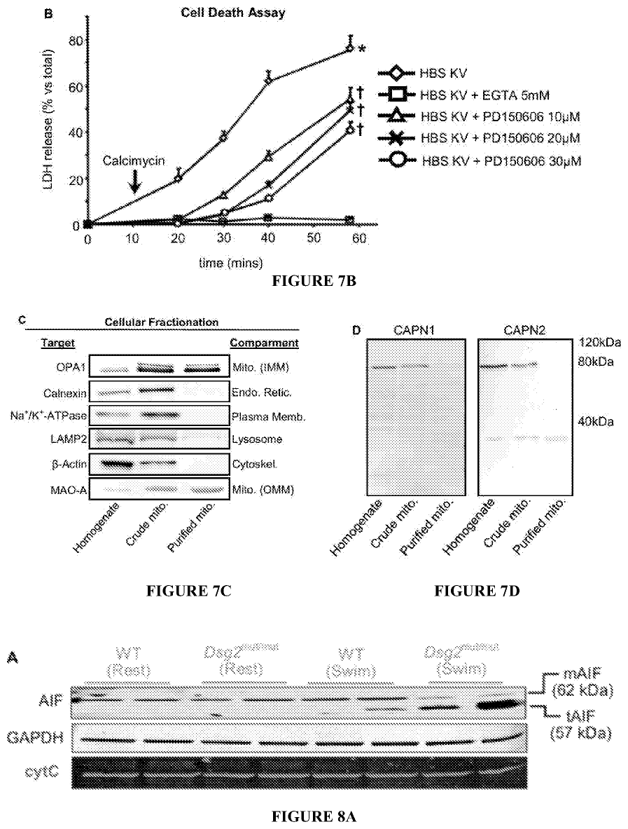

Since both calpeptin and PD150606 (to a lesser extent) restored MMP in Ca²⁺ overloaded cells, whether these inhibitors could attenuate Ca²⁺ overload-induced CAPN1 substrate hydrolysis and cell death was interrogated. Calpeptin displayed a dose-dependent reduction in Ca²⁺ overload-induced CAPN1 substrate hydrolysis (FIG. 6C) and cell death (FIG. 6D). Although PD150606 also showed a dose-dependent decrease in CAPN1 substrate hydrolysis (˜60% reduction, FIG. 7A), PD150606 did not attenuate CAPN1 activity to the same degree as calpeptin (˜75% reduction, FIG. 6C). Furthermore, PD150606 was far less effective in reducing cell death (˜15% reduction; FIG. 7B) than calpeptin (˜40% reduction; FIG. 6D). Thus, calpeptin is an efficacious CAPN1-inhibitor in preventing Ca²⁺ overload-induced mitochondrial dysfunction, CAPN1-substrate cleavage, and myocyte necrosis in HL-1 cells. HL-1 cells were subjected to Ca²⁺ overload (via Calcimycin, black arrows in FIG. 7) in HBS KV medium in the absence or presence of a PD150606 dose-dependent response.

The cytosolic and mitochondrial localization of CAPNs were next examined. Mitochondria were purified via density gradient, as confirmed by the absence of proteins from subcellular compartments, such as cytoskeleton (0-actin), lysosome (LAMP2), endoplasmic reticulum (calnexin), and plasma membrane (Na*/K+-ATPase) (FIG. 7C). Conversely, purified mitochondrial fractions were highly enriched for both inner (OPA1) and outer mitochondrial membrane (MAO-A) markers (FIG. 7C). CAPNs are polypeptide complexes, comprising an 80 kDa Ca²⁺-dependent peptide (Domains I-IV) and 28 kDa regulatory peptide (Domains V-VI). Antibodies directed towards Domain-IV of CAPN1 and CAPN2 did not display an 80 kDa band in purified mitochondrial extracts (lane 3 of both immunoblots; FIG. 7D). Thus, both CAPN1/2 are not constitutively localized in mitochondria from HL-1 cells under physiological Ca²⁺ levels.

Whether CAPNs translocate into mitochondria upon Ca²⁺ overload was then investigated. In HL-1 cells, CAPN1, but not CAPN2, was abundantly localized in mitochondrial extracts within two minutes of Ca²⁺ overload (lane 3; FIG. 6E). Interestingly, both calpeptin and PD150606 failed to prevent mitochondrial CAPN1 translocation even in the presence of Ca²⁺ overload (lanes 6 & 7; FIG. 6F), whereas Ca²⁺-chelation resulted in considerable loss of mitochondrial CAPN1 levels (lane 5; FIG. 6F). Thus, indicating that CAPN1 cytosolic-to-mitochondrial localization was Ca²⁺-dependent yet independent of enzymatic activity in HL-1 cells.

Chronic exercise increases cardiac levels of active, cleaved CAPN1 (75 kDa) in WT and DSG2^{mut/mut} mice (as seen in FIGS. 3A-3C). However, these findings and those related to mitochondrial loss of CAPN1 following Ca²⁺-chelation (FIGS. 6E, 6F, and 7D) were obtained utilizing a Domain IV-directed CAPN1 antibody. Domain IV binds Ca²⁺ to regulate CAPN1 autoproteolytic cleavage and activation, whereas Domain III uses Ca²⁺ to control CAPN1 binding to lipid bilayers. The subcellular localization of CAPN1 from exercised heart lysates with the CAPN1 clone 9A4H8D3 antibody, which specifically binds to Domain III of CAPN1 (amino acids 465-520) was then investigated. Both total (80 kDa) and active (75 kDa) CAPN1 were found in cytosolic and mitochondrial extracts from exercised WT cardiac lysates (FIGS. 6G, 6H). Yet, mitochondrial lysates from exercised Dsg2^{mut/mut} mice hearts showed the active, cleaved CAPN1 (75 kDa) peptide, more prominently (FIGS. 6G, 6H). These additional data demonstrate CAPN1 cytosolic-to-mitochondrial localization is an essential mechanistic step in exercise-induced CAPN1 mitochondrial activation and localization in ACM myocytes.

### Example 6

### CAPN1 Activation Leads to AIF Truncation

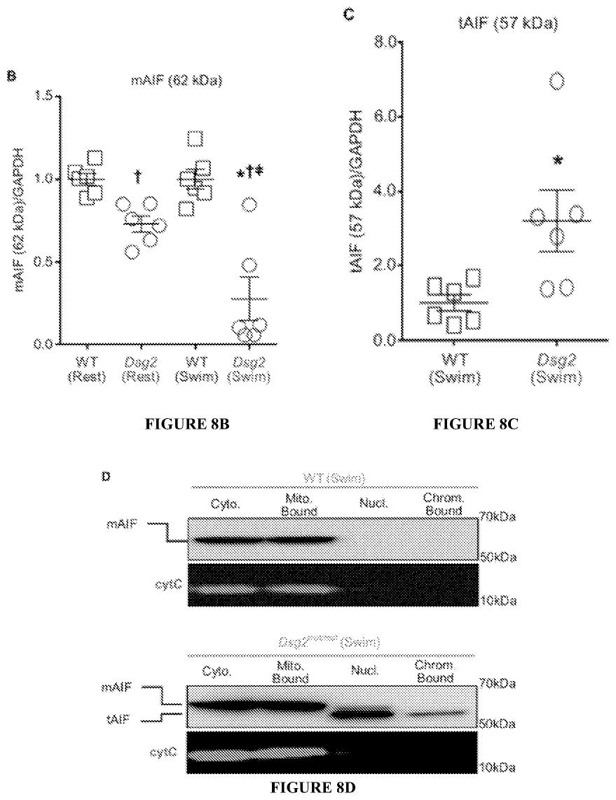

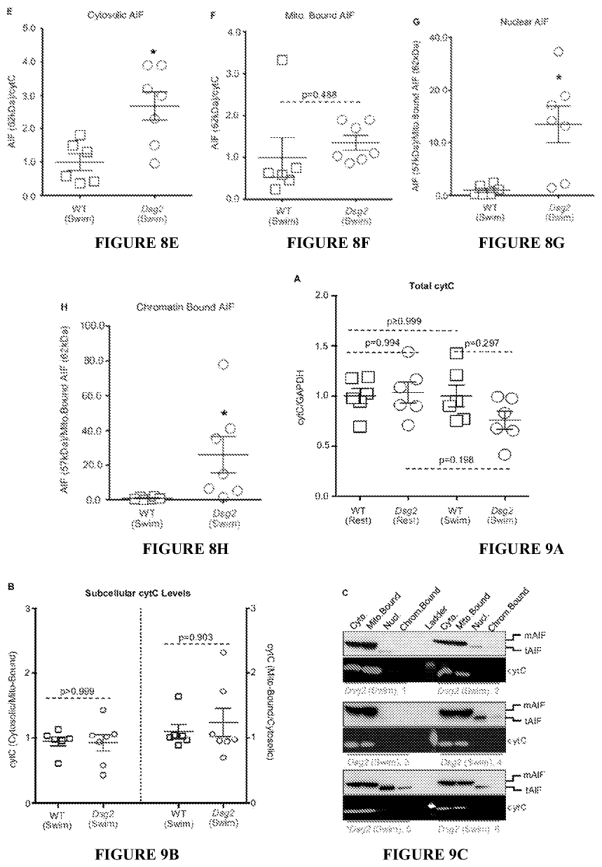

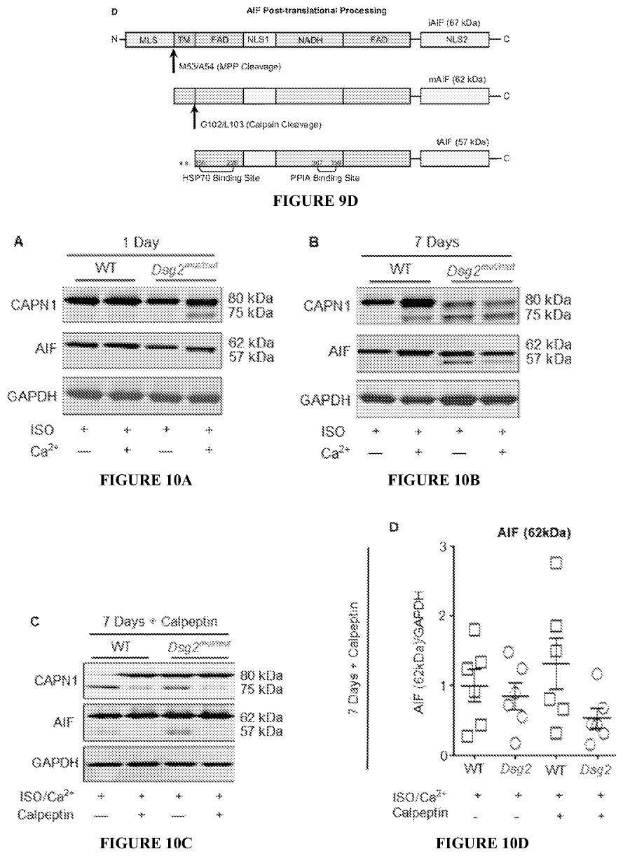

These in vivo and in vitro findings parallel similar work involving a “mitochondriocentric” signal-transducer-effector (MSTE) pathway in non-ischemic cardiomyocyte necrosis. Here, this MSTE pathway involves an elevated signal (e.g., Ca²⁺) in both HL-1 and ACM myocytes, activating a Ca²⁺-dependent transducer (e.g., CAPN1), leading to myocyte necrosis (i.e., the end effect). Yet, the potential mitochondrial effector liable for myocyte necrosis in Dsg2^{mut/mut} myocytes has yet to be determined. Therefore, myocardium from both sedentary and exercised WT and Dsg2^{mut/mut} mice was assessed for changes in total cytochrome-C (cytC) and apoptosis-inducing factor (AIF) levels (FIGS. 8A-8C and 9A, 9B), two mitochondrial proteins released in response to MMP depolarization and implicated in either apoptosis, necrosis, or both. In addition to total protein, subcellular fractionation was performed to separate (i) cytosolic; (ii) mitochondrial-bound; (iii) nuclear; and (iv) chromatin-bound protein lysates to discriminate between the cellular localization of cytC and AF (FIGS. 8D and 9C). The presence of truncated AIF (tAIF) in nuclear (Nucl.) and chromatin bound (Chrom. Bound) lysates was denoted (*denotes the immunoblot used in FIG. 9D). In total lysates, no changes in the expression of cytC between genotypes were found, both at rest and after exercise (FIGS. 8A and 9A). Importantly, although both mitochondrial-bound and cytosolic extracts harbored cytC in exercised mice (FIG. 8D), there was no sizable difference in the ratio between cytosolic:mitochondrial-bound and/or mitochondrial-bound:cytosolic cytC levels in exercised WT vs. Dsg2^{mut/mut} mice (FIG. 9B). Conversely, and regardless of genotype, exercised myocardium showed two distinct AIF bands (62 kDa and 57 kDa; FIG. 8A). Elevated intracellular Ca²⁺ levels can lead to Ca²⁺-dependent auto-proteolytic cleavage of CAPN1, activating cleaved CAPN1, where mature AF (mAIF; 62 kDa) can undergo CAPN1-mediated proteolysis (FIG. 9D). Once truncated (tAIF, 57 kDa; FIG. 9D), AIF migrates to the nucleus, triggering large-scale DNA fragmentation and cell death. Here it was found that Dsg2^{mut/mut} mice show reduced mAIF levels compared to sedentary controls, and exercise exacerbated this phenomenon (FIG. 8B). Furthermore, myocardium from exercised Dsg2^{mut/mut} mice showed elevated levels of tAIF compared to exercised controls (FIG. 8C).

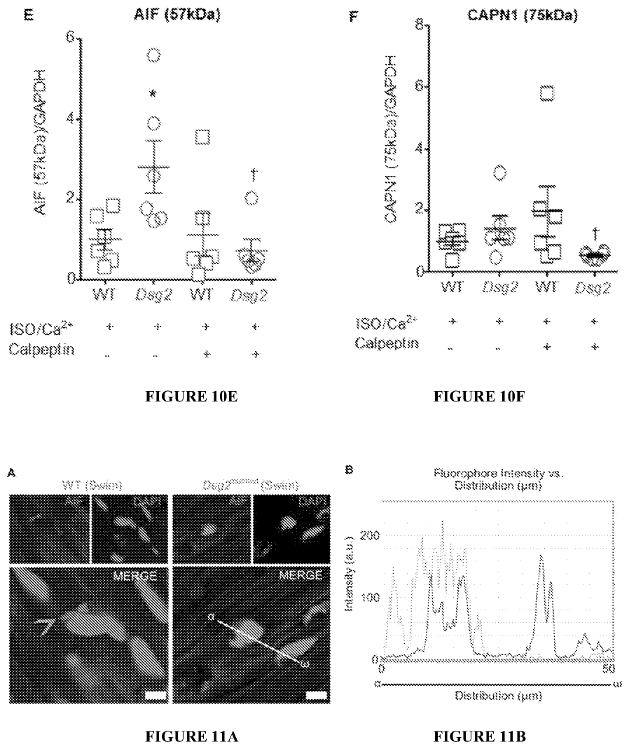

Only exercised Dsg2^{mut/mut} mice showed the presence of nuclear and chromatin-bound tAIF (i.e., 57 kDa) compared to WT counterparts (FIGS. 8G, 8H, and 9C). Building on in vivo exercise findings, whether an exogenous β-adrenergic stimulus, alone or in the presence of elevated Ca²⁺ levels, is necessary to cleave CAPN1 (i.e., 75 kDa active isoform) and drive CAPN1-mediated truncation of ATF in ACM myocytes was assessed. To test this, embryonic stem cells (ECs) from WT and Dsg2^{mut/mut} mice were established and ECs were differentiated into cardiomyocytes (ES-CMs). The resulting ES-CMs were then treated with isoproterenol (ISO; 50LM) in the absence or presence of Ca²⁺(1 μM). One-day treatment of ISO-alone and/or dual ISO/Ca²⁺ failed to induce the 75 kDa CAPN1 fragment in WT ES-CMs yet sufficient to generate activated CAPN1 in Dsg2^{mut/mut} ES-CMs, although insufficient to truncate AIF (FIG. 10A). Conversely, seven-day treatment of ISO/Ca²⁺ was sufficient to drive tAIF (i e., 57 kDa) formation in Dsg2^{mut/mut} ES-CMs, an event accompanied by the cleavage of CAPN1 (FIG. 10B). The formation of truncated AIF (57 kDa) in Dsg2^{mut/mut} ES-CMs treated with both ISO-alone and ISO/Ca²⁺ for 7 days was denoted (FIG. 10B).

Considering the efficacy of calpeptin over PD150606 on the attenuation of CAPN1 substrate hydrolysis in HL-1 cells reported above, whether calpeptin could attenuate chronic ISO/Ca²⁺-induced CAPN1 activation and AIf-truncation was assessed (FIG. 10C). Intriguingly, WT ES-CMs pretreated with calpeptin in the presence of chronic ISO/Ca-showed a slight increase in the levels of mAIF (62 kDa) as compared to Dsg2^{mut/mut} ES-CM counterparts (FIG. 10D). Pretreatment with calpeptin significantly lowered the levels of both tAIF (57 kDa) and cleaved (75 kDa) CAPN1 compared in Dsg2^{mut/mut} ES-CMs (FIGS. 10E, 10F). This evidence suggested that ACM myocytes undergoing experimental (in vitro) and environmental (in vivo) exercise are more prone to Ca²⁺/CAPN1-mediated cleavage of AIF, an effect likely due to an intrinsic mishandling of Ca²⁺ already present in Dsg2^{mut/mut} myocytes.

### Example 7

### AIF Migrates to the Myocyte Nucleus in Exercised Dsg2^{mut/mut} Mice and ACM Patients

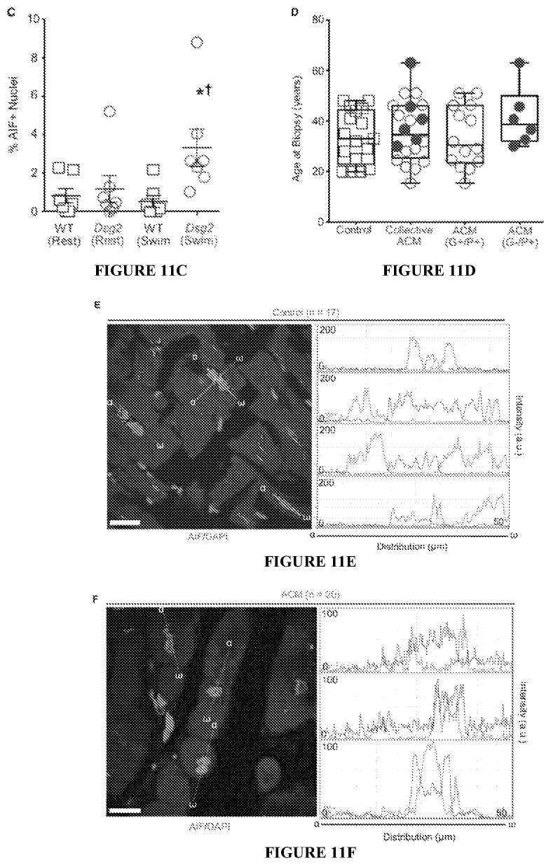

Upon cleavage, truncated AIF classically translocates to the nucleus to induce chromatin condensation and cell death. Therefore, whether AIF nuclear translocation occurs in Dsg2^{mut/mut} myocardium, as well as in ACM patients was examined. To this end, murine (FIGS. 11A-11C) and human (FIGS. 11D-11G) myocardium were analyzed for AIF-DAPI colocalization. In response to exercise, AIF-DAPI nuclear colocalization was increased in the myocardium of exercised Dsg2^{mut/mut} mice compared to WT mice and WT and ACM sedentary cohorts (FIGS. 11A-11C).

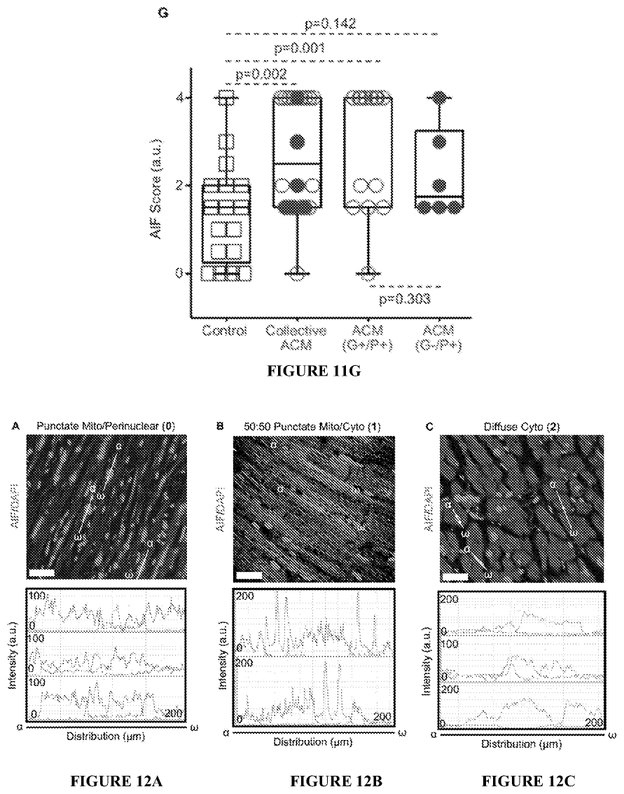

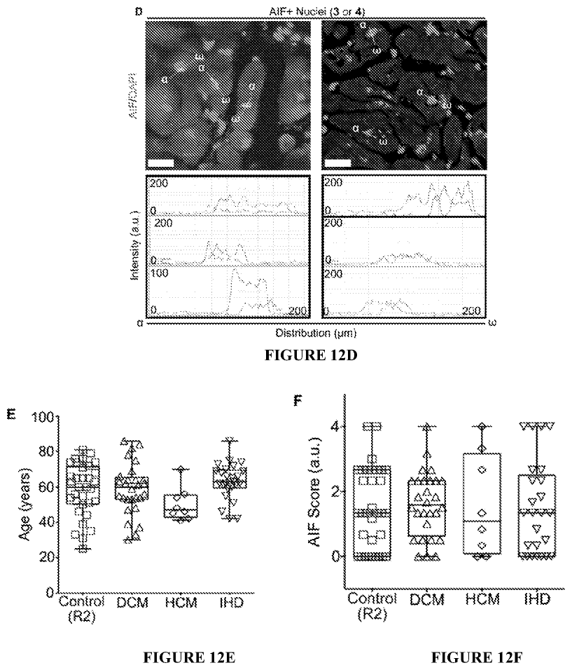

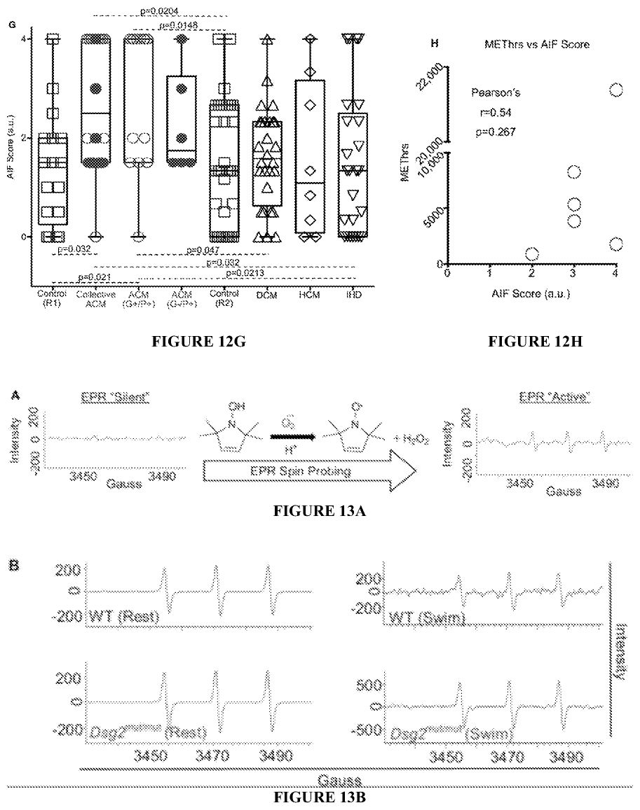

Myocardial samples from three age-matched patient cohorts were then assessed for AIF localization (FIG. 11D). The first cohort included myocardial samples obtained at autopsy from individuals with no prior clinical history of heart disease (Controls, n=17; Cohort 1). The second cohort consisted of age-matched myocardia from ACM patients bearing a known pathogenic desmosomal gene variant implicated in ACM (DSG2 [n=2] and plakophilin-2, PKP2 [n=12]; Cohort 2). The third cohort consisted of myocardia from gene-elusive ACM patients, i.e, ACM patients in whom no pathogenic desmosomal gene variant had been identified but met Task Force Criteria (TFC) for ACM at the time of biopsy collection (n=6; Cohort 3). ACM patient gene variants are detailed in Table 6. Three to ten regions of interest (ROI) were identified in each patient myocardial sample and non-pathological (Grades 0-2; FIGS. 12A-12C) and pathological (Grades 3-4; FIG. 12D) fluorophore intensity vs. fluorophore distribution (i.e., AIF-to-DAPI overlap) scores were recorded. Myocardium was given an AIF Pathological Score (Grade 0-4, number in bold within parentheses) where (A) Grade 0 represents myocardia displaying robust punctate mitochondrial (mito) and/or perinuclear AIF; (B) Grade 1 scored in myocardium with a 50:50 odds ratio of punctate mito AIF and diffuse cytosolic (Cyto) AIF; (C) Myocardia with diffuse cyto AIF and rare punctate AIF localization was scored as Grade 2; (D) Presence of AIF positive (AIF+) nuclei were scored as Grade 3 (n=2-3 AIF+ nuclei) or Grade 4 (n≥4 AIF+ nuclei).

Myocardium from both control and ACM patients showed a range of diffuse cytosolic AIF localization (Grade 2), a 50:50 odds ratio of cytosolic and mitochondrial AIF localization (Grade 1), and punctate mitochondrial and/or perinuclear localization of AIF (Grade 0) and were thus scored as non-pathological for AIF nuclear localization (FIGS. 11E-11G). However, myocardium from ACM patients, collectively (Cohorts 2 and 3), showed increased AIF positive (+) nuclei (Grades 3-4; n==11/20) compared to control myocardium (Grades 3-4; n=2/17; FIG. 11G). Considering the prominent AIF-nuclear localization found in ACM myocardium compared to age-matched controls (FIG. 11G), next whether, and to what extent, AIF-nuclear translocation occurs in other, non-ACM forms of cardiomyopathies was interrogated. To this end, a tissue microarray (TMA) comprising age-matched (FIG. 12E) LV tissue from cases of hypertrophic (HCM; n==8), dilated cardiomyopathy (DCM; n=28), ischemic heart disease (IHD; n=25), and controls (n=33) were evaluated. First, blinded analysis of samples revealed the average AIF score for the TMA control cohort (1.49±0.22, mean±SEM, n=33) was nearly identical to controls (1.35±0.28, mean±SEM, n=17, P>0.999; FIG. 12G) analyzed against ACM cohorts in FIG. 11. This indicates that AIF scoring in control samples is independent of the number of samples assessed, consistent against the different times evaluations were made, and reliable considering three different blinded observers were employed. Next, no difference in AIF scores on the TMA between the control and DCM, ICM, and IHD cohorts were observed (FIG. 12F). Whether increased AIF nuclear localization in ACM cohorts differs between non-ACM heart disease cohorts and all control samples was determined. Remarkably, increased ALF scores in ACM cohorts against controls persisted., while AIF scores were additionally increased in the myocardium of ACM subjects compared to that of DCM and HID patients (FIG. 12G). These data collectively indicate that in a small cohort of ACM patients, on average, myocardium displayed more elevated AIF+ nuclei levels than controls, DCM and-ID (FIGS. 11G and 12G).

TABLE 6
Desmosomal gene variants in ACM patients.

| | | | No. of Patients |
|---|---|---|---|
| Gene (n) | Nucleotide Change | Amino Acid Change | (n = 14) |
| PKP2 (12) | c.2509delA | p.Ser837Valfs*94 | 2 |
| | c.148_151delACAG | p.Thr50Serfs*61 | 3 |
| | c.2146 − 1G > C | mutant splice product | 1 |
| | c.2013delC | p.Pro672fs*12 | 2 |
| | c.2489 + 1G > A | mutant splice product | 2 |
| | c.2197_2202delCACACCinsG | p.His733Alafs*8 | 1 |
| | c.235C > T | p.Arg79* | 1 |
| DSG2 (2) | c.523 + 2T > C | mutant splice product | 1 |
| | c.918G > A | p.Trp306* | 1^{a} |
| | c.146G > A | p.Arg49His | |

PKP2, Plakophilin-2;
DSG2, Desmoglein-2;
^{a}Indicates patient with a compound heterozygote mutation in DSG2

### Example 8

### AIF-Pathological Scores Correlate with a Pathogenic Desmosomal Gene Variant

Whether a correlation exists between pathological AIF-immunostained myocardium and the presence of a pathogenic desmosomal gene variant within ACM cohorts was interrogated. Marked AIF pathological scores were found (FIG. 11G) only in myocardial samples from patients who met TFC for ACM (phenotype positive, P+) and harbored a pathogenic desmosomal gene variant (genotype positive, G+). Conversely, no difference in myocardial AIF-pathological scores were apparent between controls and patients who met TFC for ACM yet harbored no pathogenic desmosomal gene variant (G−/P+[i.e., gene elusive ACM patients]) (FIG. 11G). No difference in AIF-pathological scores were additionally observed between gene-elusive and gene-positive ACM patients (FIG. 11G). Therefore, it was interrogated if clinical phenotypes accounted for the discrepancy between gene-elusive and gene-positive ACM patients. Pearson's correlation analyses were performed for AIF pathological scores against proband status, major or minor TFC for structural involvement, repolarization and depolarization abnormalities, and arrhythmic burden between ACM cohorts (Table 7). While ACM cohorts, when taken collectively (i.e., both G−/P+ and G+/P+ ACM patients), displayed increased AIF-nuclear localization compared to controls (FIG. 11G), a stronger correlation between AIF-pathological scores emerged only in those ACM individuals bearing a pathogenic desmosomal gene variant (FIG. 11G). Lastly, whether exercise contributed to the higher AIF+ nuclei content in ACM patient myocardium was evaluated. Retrospective exercise participation reports were reviewed from six ACM patients with both myocardial samples immunostained for AIF and exercise histories and analyzed metabolic equivalent of task hours (METhrs) against AIF scores (FIG. 12H). Pearson's correlation analyses for AIF cores against METhrs showed a positive trend, albeit not significant (Pearson's r=0.54, p=0.267; FIG. 12H).

TABLE 7
Clinical characteristics and AIF-pathology scores in ACM patients.

| ACM Cohort | Overall (n = 20) | Desmosomal (14) | TFC (6) | P-value |
|---|---|---|---|---|
| AIF-immunostained score, mean ± SEM | 2.85 ± 0.3 | 3.11± 0.3 | 2.25 ± 0.4 | 0.474 |
| Clinical Phenotypes at Collection | Overall (n = 20) | Desmosomal (14) | TFC (6*) | P-value |
| Proband, n (%) | 13 (65) | 19 (64) | 4 (67) | >0.999 |
| Structural alterations, n (%) | 12 (60) major | 10 (71) major | 2 (33) major | >0.999 |
| | 5 (25) minor | 2 (14) minor | 3 (50) minor | |
| Repolarization abnormalities, n (%) | 5 (25) major | 4 (29) major | 1 (17) major | >0.999 |
| | 9 (45) minor | 6 (43) minor | 3 (50) minor | |
| Depolarization abnormalities, n (%) | 17 (85) major | 14 (100) major | 3 (50) major | 0.297 |
| | 0 (0) minor | 0 (0) minor | 0 (0) minor | |
| Arrhythmias, n (%) | 7 (35) major | 6 (43) major | 1 (17) major | 0.950 |
| | 10 (50) minor | 7 (50) minor | 3 (50) minor | |

### Example 9

### Dsg2^{mut/mut} Myocardium Displays Lower Antioxidant Capacity Via the Thioredoxin System

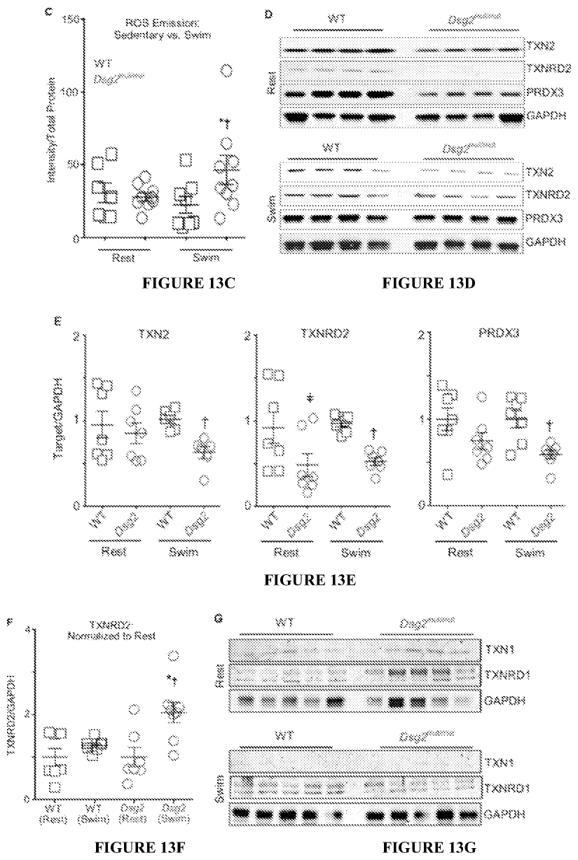

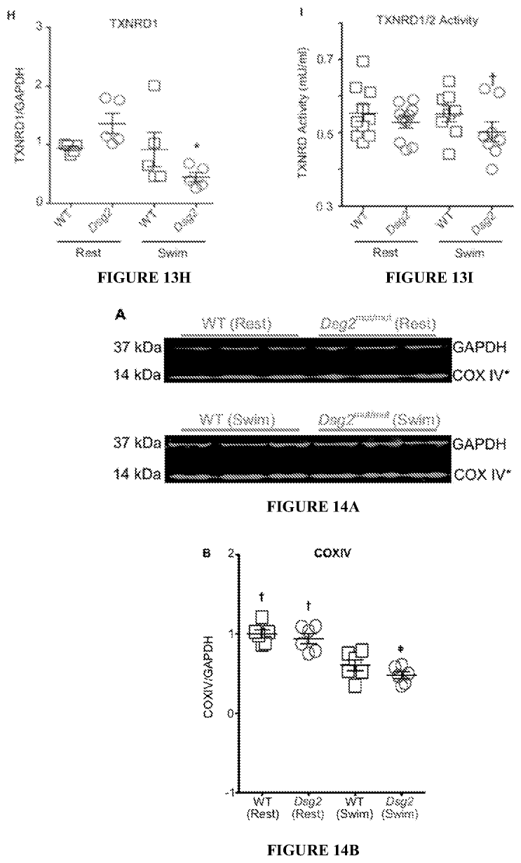

Physical effort increases ROS production, and if not adequately scavenged, it can lead to myocardial inflammation, fibrosis, and ultimately cell death. The mitochondrial thioredoxin-2 (TXN2) system is essential for cell viability and a significant regulator of H₂O₂ emissions from the mitochondria. Whether endurance exercise contributes to the destabilization of Dsg2^{mut/mut} mitochondria stemming from increased ROS emission due, at least in part, to a flawed TXN2 system was evaluated. Sedentary mice, regardless of genotype, displayed similar ROS levels at rest, as determined by electron paramagnetic resonance (EPR) spectroscopy (FIGS. 13A-13C). Conversely, swimming augmented ROS emission in Dsg2^{mut/mut} myocardium, an effect not manifested in exercised WT mice (FIG. 13C). Compared to WT mice, sedentary Dsg2^{mut/mut} mice displayed a trend towards reduced TXN2 levels and markedly reduced end-effector levels peroxiredoxin-3 (PRDX3), a mitochondrion-specific peroxidase (FIGS. 13D, 13E). Strikingly, TXN2 reductase (TXNRD2) myocardial expression was nearly absent in sedentary mutants (FIGS. 13D, 13E). Knowing that myocardial TXNRD2 expression is upregulated in response to physical training in healthy rodents, whether swimming elevated TXNRD2 expression in exercised cohorts was tested. Although TXNRD2 expression levels remained considerably lower in the myocardium from exercised Dsg2^{mut/mut} mice compared to exercised WT mice (FIGS. 13D, 13E), TXNRD2 expression was indeed elevated in response to exercise (FIGS. 13D, 13F). Therefore, TXNRD2 levels from exercised cohorts were normalized against their respective sedentary cohort TXNRD2 levels (FIG. 13F). Myocardial samples from exercised Dsg2^{mut/mut} mice displayed increased TXNRD2 expression compared to exercised WT mice and sedentary mutants (FIG. 13F). Increased TXNRD2 levels from exercised Dsg2^{mut/mut} mice compared to TXNRD2 levels from sedentary Dsg2^{mut/mut} mice was noted. Interestingly, exercise led to a further decline in TXN2 and PRDX3 content in ACM mice (FIGS. 13D, 13E).

Mitochondrial ROS can induce further ROS release from cytosolic sources and cytosolic thioredoxin-1 (TXN1) inhibition leads to myocardial oxidative damage. Therefore, the status of cytosolic TXN1 and TXN1 reductase (TXNRD1) was evaluated. Regardless of genotype, both sedentary and exercise cohorts displayed similar TXN1 content (FIG. 13G). Although TXNRD1 levels were upregulated in sedentary ACM mutants, swimming resulted in reduced TXNRD1 protein (FIGS. 13G, 13H). Finally, whether TXNRD1/2 expression was associated with TXNRD1/2 activity (FIG. 13I) was tested. Myocardial homogenates were incubated with NADPH and a disulfide substrate, either in the presence or absence of the TXNRD1/2 inhibitor, auranofin. Although mutants showed a trend towards reduced TXNRD1/2 activity under resting conditions, only exercised Dsg2^{mut/mut} mice demonstrated a marked decline in TXNRD1/2 activity compared to exercised controls (FIG. 13I). Of note, myocardial lysates from exercised Dsg2^{mut/mut} mice showed reduced TXNRD1/2 activity compared to exercised WT mice. Thus, in response to exercise, the mitochondrial and cytosolic TXN systems' inadequate levels lead to augmented ROS emissions in Dsg2^{mut/mut} hearts, likely contributing to mitochondrial dysfunction and CAPN1 mitochondrial access.

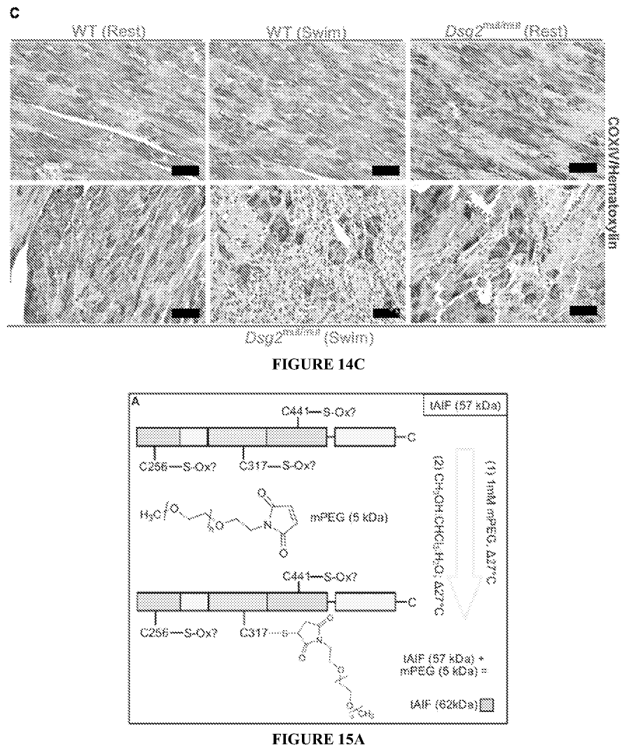

A loss in AIF function and/or levels has been shown to augment ROS emission and reduce COXIV expression. ROS levels were shown as elevated in the hearts of Dsg2^{mut/mut} mice (as seen in FIGS. 13B, 13C); therefore, COXIV levels via immunoblotting and immunoperoxidase staining between cohorts and genotypes were assessed. In contrast to prior reports demonstrating exercise-induced elevation in COXIV expression, a reduction in COXIV, regardless of genotype (FIGS. 14A, 14B) was found. Conversely, exercise reduced the levels of COXIV in both WT and Dsg2^{mut/mut} mice. This unexpected finding prompted to examine COXIV localization, using a sensitive COXIV immunoperoxidase stain. a drastic reduction in COXIV myocardial localization in exercised Dsg2^{mut/mut} mouse myocardium, particularly in areas harboring extensive myocardial damage and/or inflammatory infiltration was observed (FIG. 14C). It was noted, a diffuse COXIV localization throughout myocardia from sedentary and exercised WT mice and sedentary Dsg2^{mut/mut} mice (top panels of FIG. 14C). While exercised Dsg2^{mut/mut} mouse myocardium showed areas of diffuse COXIV localization (left bottom panel), numerous areas of absent COXIV expression were found around infiltrative inflammatory cells (bottom two right panels, dashed lines). Considering that COXI-COXIII are encoded by mitochondrial DNA, whereas nuclear DNA encodes COXIV-COXVII, the drastic decline in COXIV levels and abnormal COXIV tissue localization observed in Dsg2^{mut/mut} hearts may stem from a nuclear insult, which accounts for reduced DNA-mediated transcription of COXIV.

### Example 10

### Oxidized-tAIF Induces DNA Fragmentation

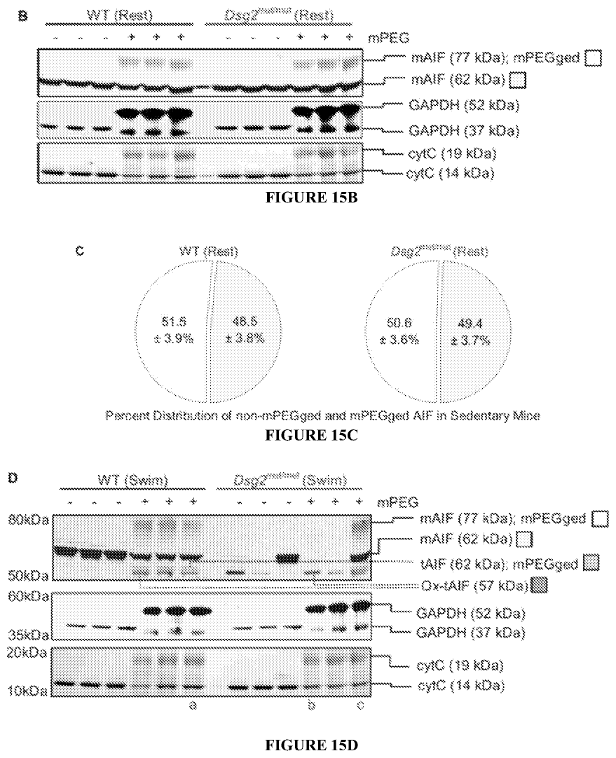

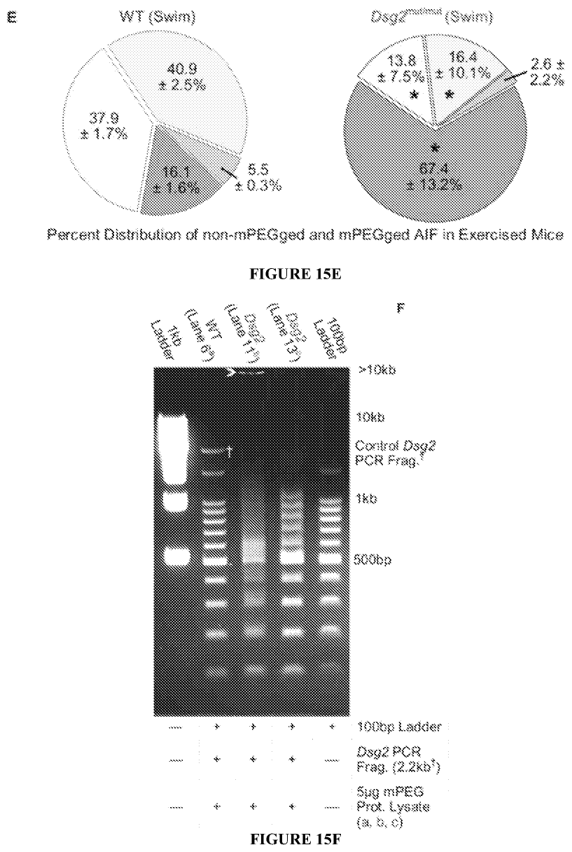

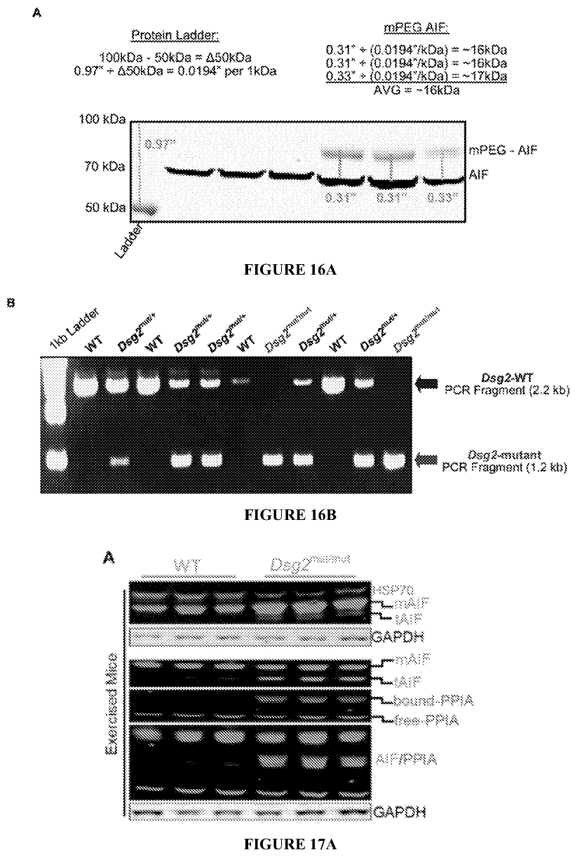

Whether in its mature or truncated form, AIF contains three cysteinyl residues (C256, C317, and C441; FIG. 15A), and it was showed that AIF undergoes oxidative modification prior to CAPN1-proteolytic processing and mitochondrial release. Considering only exercised myocardium displayed tAIF, the status (oxidized vs. reduced) of AIF cysteines was tested. Specifically, mitochondrial-bound fractions from sedentary and exercised lysates were treated with a 5 kDa cysteine-labeling agent, methoxy polyethylene glycol maleimide (mPEG; FIG. 15A). Any non-oxidized cysteine (C256, C317, and/or C441) binds mPEG, adding a 5 kDa weight to AIF per cysteine bound-mPEG. When mPEG binds to each cysteine in the reduced state, it generates a 5 kDa shift from the original molecular weight of AIF per mPEG-bound cysteine (FIGS. 15A and 16A). In FIG. 16A, the difference in 100-50 kDa protein ladder (Δ50 kDa) divided by the length (in inches) between 100-to-50 kDa bands (0.97″) was used to formulate the weight of mPEGged AIF. AIF contains 3 cysteines (regardless of its mature or truncated form). Representative immunoblot from WT mice (rest) showed that mPEG bound to each free cysteine of AIF, thus generated a −15 kDa shift in protein weight. More specifically, all three cysteines were not oxidized. Thus, only non-oxidized, (i.e., reduced) cysteines can bind mPEG. When tested in insoluble lysates containing mitochondrial-bound fractions, mPEG bound to all three available cysteines in AIF from sedentary cohorts, regardless of genotype (FIGS. 15B, 15C). In addition to the findings seen in FIG. 8, this evidence confirms that mitochondrial-bound AIF is in a reduced and intact state (i.e., not oxidized or truncated) in sedentary mutant mice. Conversely, in exercised mutants, mitochondrial-bound fractions showed the presence of a truncated (tAIF) and oxidized (ox) form of tAIF (i.e., ox-tAIF; FIGS. 15D, 15E). Of note, there was a ˜4-fold increase in the levels of non-mPEGged (i.e., cysteines have been oxidatively [Ox] modified) Ox-tAIF (57 kDa, red) in DSG2^{mut/mut} mice compared to Ox-tAIF (57 kDa, red) in myocardial lysates from exercised WT mice. For FIGS. 15C and 15E, percent distribution is calculated as the expression of each individual mPEGged and/or non-mPEGged AIF levels divided by the sum of all AIF levels times 100.

Prior studies indicate DNA binding is a requirement for AIF-induced cell death, and large-scale DNA fragmentation is a biochemical hallmark of AIF-driven cell death. Therefore, whether myocardial ox-tAIF from exercised Dsg2^{mut/mut} mice promotes DNA fragmentation via a DNA-retardation assay was evaluated. First, the wildtype Dsg2 DNA fragment was generated via polymerase chain reaction (PCR) that is utilized to genotype WT vs Dsg2-mutant mice (2.2 kb Dsg2-WT fragment vs 1.2 kb Dsg2-mutant fragment, respectively; FIG. 16B. 5p g of insoluble lysates from mPEG treated samples (tAIF vs ox-tAIF lysates) were incubated in the presence of a 100 bp DNA ladder (ranging from 100 bp to 1.5 kb) and the 2.2 kb control Dsg2 DNA fragment (FIG. 15F). In FIG. 16B the control Dsg2-WT DNA fragment runs at 2.2 kb, and the Dsg2-mutant fragment runs at 1.2 kb on a 1.5% agarose gel. Mice homozygous for mutant Dsg2 (Dsg2^{mut/mut}) are labeled in red, mice heterozygous for mutant Dsg2 (Dsg2^{mut/+}) are labeled in black, and WT mice are labeled in blue. DNA samples containing insoluble lysates that harbored ox-tAIF began to show the presence of DNA retardation as early as ˜200 bp (lane 3; FIG. 15F). Conversely, lysates containing non-oxidized tAIF began to retard DNA at ˜500 bp (lane 4; FIG. 15F). Furthermore, non-oxidized tAIF lysates showed DNA retardation from 500 bp to 1,000 bp, yet these bands were visibly distinct from one another (lane 4; FIG. 15F). In stark contrast, ox-tAIF lysates showed DNA retardation from 500 bp and upward, no distinct DNA laddering after 500 bp, and complete prevention of the migration of DNA fragments above 1,000 bp (lane 3; FIG. 15F). Taken together, these results indicate that in the myocardium of exercised ACM mice, AIF is truncated by CAPN1, but also oxidized. The latter effect is due, at least in part, to the downregulation of the TXN system leading to ox-tAIF-mediated DNA fragmentation. Five micrograms [5 μg] of mPEG-treated protein lysates (e.g., FIG. 15D: lane 6 [a]; lane 11 [b]; and lane 13 [c]) were incubated with a 2.2 kb DNA fragment (?Frag.) generated via polymerase chain reaction (PCR) from the WT form of murine Dsg2 (see FIG. 14B) and a 100 bp DNA ladder. Of note, lysates containing Ox-tAIF (lane 11, [b]) showed DNA retardation ˜300 bp, no distinct DNA laddering after 500 bp, and complete prevention of DNA migration of any DNA fragment over >2 kb (yellow arrowhead). In contrast, lysates containing non-oxidized tAIF (lane 13, [c]) showed DNA retardation ˜500 bp and distinct DNA laddering was still visible from 500-1,000 bp.

### Example 11

### Targeting PP1A Prevents Aif-Nuclear Import, Myocyte Apoptosis, and Nuclear HMGB1 Release in ACM Myocytes Challenged with Sustained B-Adrenergic Stimulation/Ca²⁺ Overload

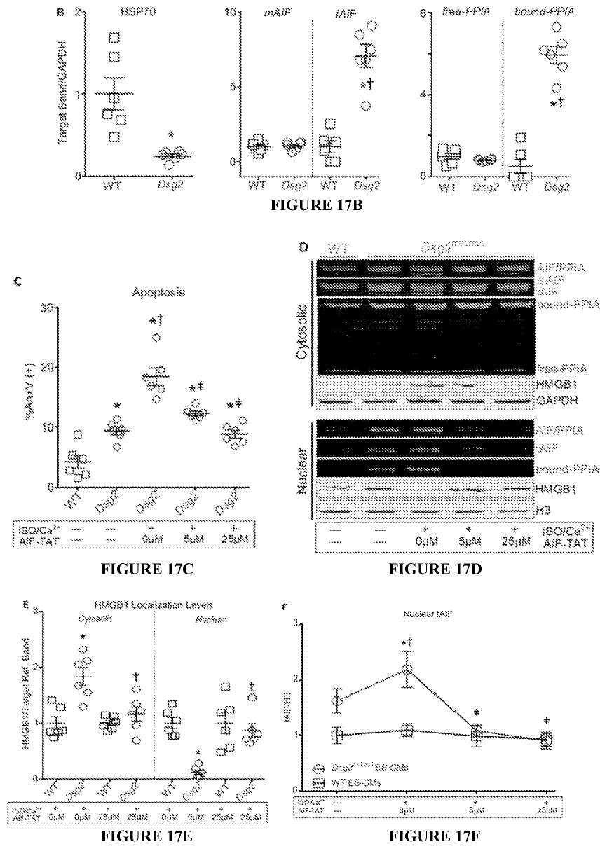

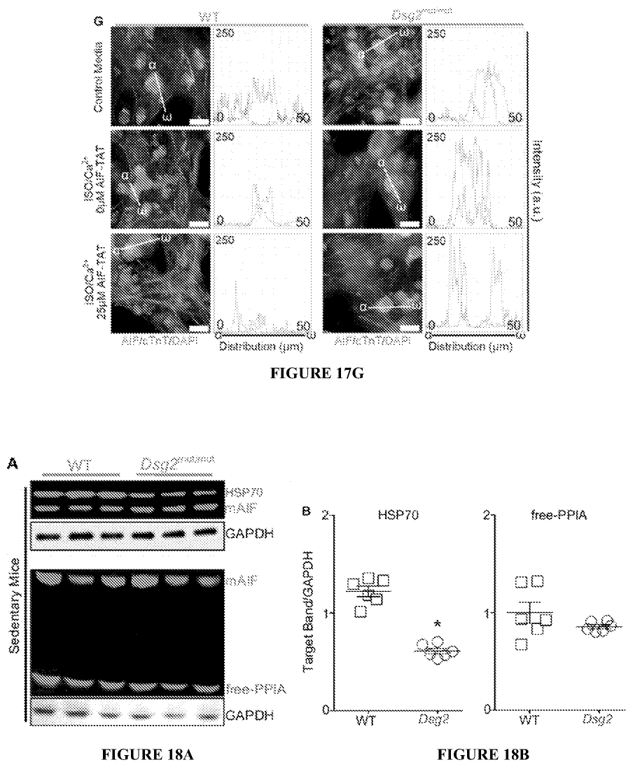

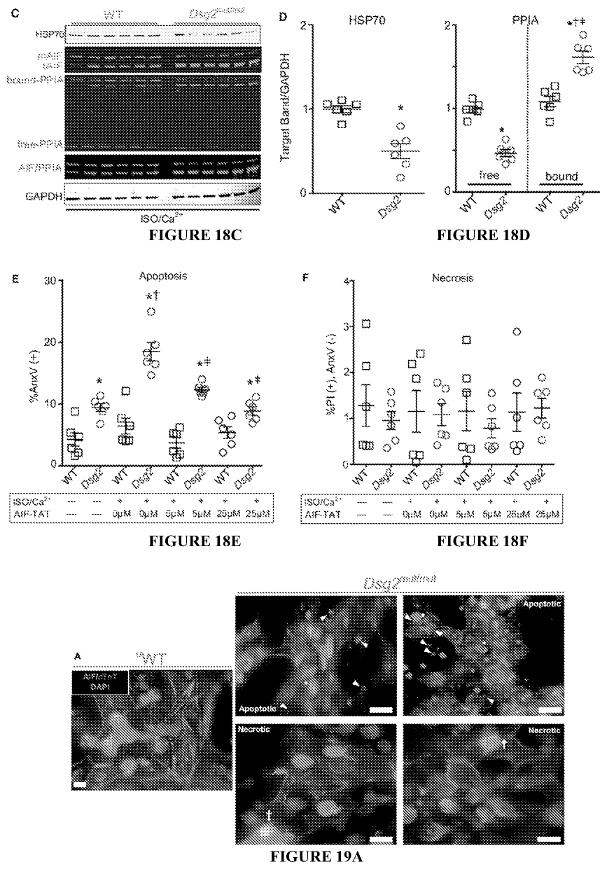

An inhibitor targeting the AIF putative DNAse activity sequence would be an ideal approach in preventing AIF-mediated DNA fragmentation and cell death, yet this DNAse site has yet to be identified. Previous research has shown that heat shock protein-70 (HSP70) is the endogenous inhibitor of AIF nuclear import. Therefore, the levels of HSP70 in the hearts of sedentary and exercised mice and ISO/Ca²⁺ stimulated ES-CMs were evaluated. Interestingly, while the levels of HSP70 were significantly reduced in sedentary (FIGS. 18A, 18B) and exercised (FIGS. 17A, 17B) Dsg2^{mut/mut} mice and ISO/Ca²⁺ treated Dsg2^{mut/mut} ES-CMs (FIGS. 18C, 18D) compared to respective controls, HSP70 did not migrate or colocalize with mature (mAIF) and/or truncated AIF (tAIF) levels (FIGS. 18A-18D; FIGS. 17A-17B). Dsg2^{mut/mut} mice HSP70 levels did not colocalize with mature (mAIF) or truncated (tAIF). However, PPIA colocalized with tAIF in exercised Dsg2^{mut/mut} mice. This intriguing finding suggests ACM myocytes inherently express lower levels of the endogenous AIF nuclear inhibitor, yet HSP70 may not act as a primary AIF nuclear antagonist in ACM.

Cyclophilin-A (peptidyl-prolyl cis-trans isomerase, [PPIA]) is the nuclear chaperone of AIF, and inhibiting PPIA binding to AIF prevents AIF-mediated cell death. Hence, the levels of free and AIF-bound PPIA in ACM cardiomyocytes were assayed. Regardless of genotype, sedentary mice showed no changes in free, native PPIA with a complete lack of AIF-bound PPIA present in myocardial lysates (FIGS. 18A, 18B). Conversely, observations in PPIA levels and PPIA colocalization with AIF were quite striking in ACM myocytes subjected to chronic exercise or ISO/Ca²⁺ stimuli. Sedentary Dsg2^{mut/mut} mice showed reduced HSP70 levels compared to WT controls. HSP70 and/or PPIA did not colocalize with AIF. Specifically, myocardium from exercised Dsg2^{mut/mut} mice showed significantly augmented bound-PPIA levels that localized with tAIF, while this observation was not apparent in exercised WT mice (FIGS. 17A, 17B). Surprisingly, the levels of free-PPIA were identical in exercised cohorts (FIGS. 17A, 17B), yet levels of free-PPIA were reduced in ISO/Ca²⁺ challenged ACM ES-CMs (FIGS. 18C, 18D). Although both WT and Dsg2^{mut/mut} ES-CMs harbored elevated bound-PPIA levels that colocalized with both mAIF and tAIF, this observation was more apparent in tAIF-bound PPIA levels from Dsg2^{mut/mut} ES-CMs (FIGS. 18C, 18D). Dsg2^{mut/mut} ES-CMs display reduced free-PPIA and increased bound-PPIA compared to WT ES-CMs.

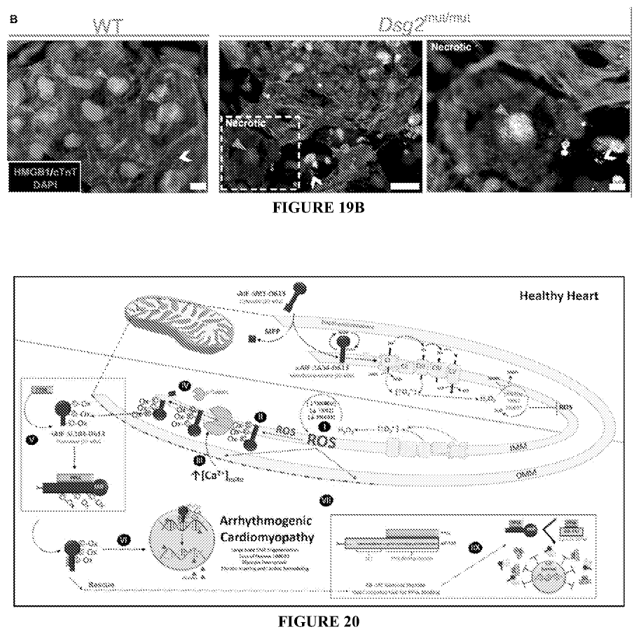

Therefore, an AIF mimetic peptide (amino acids (a.a.) 370-394), mirroring the PPIA binding domain of AIF (a.a. 367-399) that binds and sequesters cytosolic PPIA (K_{D}=1.2×10⁵) was utilized to disrupt the PPIA/AIF complex. To increase cellular uptake, the AIF-mimetic peptide was fused to the cell-penetrating, human immunodeficiency virus transactivator of transcription (TAT) fragment at its N-terminus (henceforth called, “AIF-TAT mimetic peptide”). In unstimulated cell cultures, Dsg2^{mut/mut} ES-CMs already showed elevated apoptotic levels compared to WT ES-CMs that were exacerbated in ISO/Ca²⁺ ES-CM cultures (FIGS. 17C, 18E). In FIGS. 17C-17G, ES-CMs were treated for 7 days with 50 μM isoproterenol (ISO) and 1 μM calcium (Ca²⁺), in the presence or absence of 0 μM, 5 μM, or 25 μM AIF-TAT mimetic peptide (AIF-TAT). Apoptosis in ISO/Ca²⁺-treated Dsg2^{mut/mut} ES-CMs was attenuated in ES-CMs treated with AIF-TAT. Regardless of genotype, no differences in necrotic cell death were observed (via FACS analysis) in ES-CM cultures at baseline or in ISO/Ca²⁺-treated ES-CMs (FIG. 18F). However, a sizable loss of nuclear HMGB1 was found coupled with cytosolic HMGB1 accumulation in Dsg2^{mut/mut} ES-CMs (FIGS. 17D, 17E), indicative of active necrosis. The formation of tAIF after 7 days of ISO/Ca²⁺ in Dsg2^{mut/mut} ES-CMs, which colocalized with PPIA (bound-PPIA) was noted. HMGB1 levels in ISO/Ca²⁺-treated Dsg2^{mut/mut} ES-CMs showed increased cytosolic and decreased nuclear, which was prevented in ISO/Ca²⁺-treated Dsg2^{mut/mut} ES-CMs by 25p M AIF-TAT. Yet, even in the presence of sustained β-stimulation/Ca²⁺ load, the AIF-TAT mimetic peptide considerably reduced apoptotic levels (FIGS. 17C, 18E), precluded PPIA-mediated nuclear import of tAIF (FIG. 17D, 17F), and tAIF nuclear localization (FIGS. 17G, 19A) in Dsg2^{mut/mut} ES-CMs. It was noted striated ES-CM morphology in WT ES-CMs, with cell shrinkage, rounded cell morphology, and apoptotic nuclei budding in Dsg2^{mut/mut} ES-CMs, indicative of apoptosis. Arrows, nuclear apoptotic bodies (i.e., budding). Asterisk, AIF+ nuclei. αImage is from FIG. 17G. In contrast, the lower two panels show AIF+ nuclei in Dsg2^{mut/mut} ES-CMs that show distinct signs of necrosis, such as cell dendritic extensions, swollen cell membranes and enlarged nuclei.

Interestingly, numerous AIF+ nuclei were found in Dsg2^{mut/mut} ES-CMs that showed distinct cell membrane swelling with enlarged nuclei, indicative of necrosis (FIG. 19A); and ES-CMs with robust HMGB1+ nuclei (suggesting healthy CMs) yet found in ES-CMs undergoing cellular swelling (FIG. 19B). It was noted striated ES-CM morphology in WT ES-CMs; and in the white dashed box, although Dsg2^{mut/mut} ES-CM is HMGB1+ for nuclear staining, there is cell membrane swelling and enlargement of the nucleus, indicative of necrosis. Although these observations were not accompanied by a rise or reduction in necrosis levels (FIG. 18F) assessed via FACS, it demonstrates that Dsg2^{mut/mut} ES-CMs treated with AIF-TAT mimetic peptide retained HMGB1 levels within the nucleus (FIGS. 17D, 17E). Whereas untreated, ISO/Ca²⁺-stimulated Dsg2^{mut/mut} ES-CMs cultures showed elevated cytosolic HMGB1 levels (FIGS. 17D, 17E) and extracellular HMGB1 localization (FIG. 19B). Altogether, these data indicate that AIF plays a role in caspase-independent necroptosis, involving CAPN1/PPIA/AIF-mediated apoptosis and subsequent necrosis.

### Example 12

### Discussion

In all cardiomyopathies, regardless of etiology, myocyte death is a significant determinant of cardiac dysfunction and HF. In ACM, exercise can increase the risk of SCD and pathological progression. Described herein are four previously unrecognized mechanisms accounting for exercise-triggered myocyte necroptosis in ACM: (a) Ca²⁺-overload and/or depleted CAST levels associated with elevated total and active CAPN1 levels; (b) perturbed mitochondrial membrane potential and redox assets, (c) CAPN1-dependent cleavage of oxidized-tAIF and translocation of oxidized-tAIF to the myocyte nucleus where it initiates large-scale DNA fragmentation; and (d) PPIA-mediated nuclear import of tAIF resulting in elevated apoptosis and necrotic HMGB1 release.

Very low chronic levels of apoptosis are sufficient to cause a lethal dilated cardiomyopathy, and myocyte cell death is a recurrent pathological hallmark in ACM. Mounting evidence confirms that necrosis can also be a regulated process, and very often, apoptosis and necrosis intersect in cardiac disease/remodeling. The fact that Dsg2^{mut/mut} mice harbor extensive biventricular fibrosis prompted to determine whether programmed necrosis also contributes to myocyte loss in Dsg2^{mut/mut} hearts in response to endurance exercise.

Calpain-1: a new pathogenic factor in ACM

Calpains mediate the proteolytic cleavage of a wide array of proteins involved in many physiological processes. Dysregulation of CAPN1 activity has been associated with sarcomere protein degradation leading to LV decompensation, the development of hypertension, atherosclerosis, myocardial infarction, and pressure overload, yet never investigated in ACM. Of relevance, when CAPN1 activation is potentiated, pathological phenotypes associated with the loss of Ca²⁺ control are exacerbated, as in the case of ischemia-reperfusion (I/R) injury. Here, ACM is added to this palette of pathological cardiac conditions, demonstrating that CAPN1 is activated in response to exercise in Dsg2^{mut/mut} hearts, by increased Ca²⁺ load and reduced CAST levels. In aggregate, the data indicate a cause-effect nexus between prolonged/elevated intracellular Ca²⁺ levels, CAPN1 activation, CAPN1 migration to the mitochondria, and, ultimately, necrotic cell death in HL-1 cells; events that the cell-permeable CAPN1 inhibitor, calpeptin can prevent. Of relevance, in Dsg2^{mut/mut} ES-CMs, calpeptin prevented CAPN1 activation and blocked the formation of truncated AIF even in the presence of 0-adrenergic stimuli and elevated Ca²⁺ concentrations. Accordingly, in addition to conferring protection against I/R injury and cardiac hypertrophy, CAPN1 inhibitors may serve as therapeutic agents in ACM. Mitochondrial dysfunction plays a pivotal role in cell death, and mitochondrial alterations are related to [Ca²⁺ ]i overload during I/R injury. In isolated mitochondria, Ca²⁺ addition directly alters mitochondrial function and permeability transition. However, these alterations occur at [Ca²⁺ ]i in the millimolar range, thus far from operating conditions in viable cardiomyocytes. Therefore, the link between [Ca²⁺ ]i elevation and mitochondrial derangements is likely the result of Ca²⁺-dependent processes. Here, it was revealed that CAPN1 activation is one causal linchpin between persistently elevated Ca²⁺ levels and mitochondrial dysfunction in Dsg2^{mut/mut} myocytes. Intriguingly, CAPN1 and/or CAPN2 were undetectable in mitochondria isolated under physiological Ca²⁺ levels, yet only two minutes of Ca²⁺-overload exposure was sufficient to observe a substantial mitochondrial fraction of CAPN1 (but not CAPN2). Thus, intracellular CAPN1 redistribution precedes the fall in mitochondrial membrane potential. Reports have already described the mitochondrial localization of CAPN members. However, this is the first direct observation of mitochondrial CAPN1 translocation following Ca²⁺ overload that occurs independently of CAPN1 activity, as indicated by the inability of calpeptin to prevent CAPN1 mitochondrial localization. Previous studies conducted in reperfused myocardium show CAPN binding to the sarcolemma also occurs in the presence of the CAPN inhibitor, MDL-28170. Additional studies warrant the mechanisms underlying CAPN1 translocation into mitochondria; however, it is tempting to speculate that the relationship between [Ca²⁺ ]i elevation and CAPN1 redistribution relates to covalent changes of the protease. In particular, the increase in [Ca²⁺]i might cause CAPN1 dephosphorylation by means of calcineurin activation.

Lastly, CAST is both an endogenous inhibitor and substrate of CAPN1; thus, degradation of any CAST isoforms eradicates the regulatory role of CAST and promotes CAPN1 release. There are intriguing physiological and cell-specific implications of the different CAST isoforms observed in Dsg2^{mut/mut} mice. It was previously reported that myocardial, innate NF_{K}B signaling and infiltrating T-cells in Dsg2^{mut/mut} mice leads to a cytokine storm resulting in myocardial inflammation. These new findings showing increased 70 kDa CAST isoform may shed light on an extrinsic (i.e., T-cells) role of CAPN1 activation in cardiac injury in ACM. Prior research shows that T-cells express CAPN1 and CAST even during quiescence (Go state), yet in response to antigenic stimuli and/or elevated Ca²⁺, they can proliferate and promote neighboring cell death. It has been previously demonstrated that inhibiting CAPN1 in T-cells reduces T-cell proliferation and the number of divisions T-cells can undergo and inhibits their ability to amount a pro-inflammatory cytokine surge. The role of infiltrating professional immune cells in the pathogenesis of ACM was recently detailed. Specifically, hearts from Dsg2^{mut/mut} mice showed infiltrating macrophages and T-cells and increased inflammatory cytokine expression. Therefore, while it cannot be ruled out that 70 kDa CAST isoforms may originate from uncleared erythrocytes, the possibility that they arise from infiltrating T-cells can be advanced.

CAPN1-PPIA-AIF-Mediated Necroptosis: One Modality of Myocyte Death in ACM

Mitochondrial perturbations can be a main step leading to a programmed necrotic pathway (PNP), such as caspase-independent AIF-parthanatos or caspase-independent AIF-necroptosis. Mechanistically, these two PNPs differ in how AIF-mediated cell death occurs. In AIF-parthanatos, PAR polymers generated by PARP-1 release non-truncated AIF (62 kDa) from an outer mitochondrial pool. This death modality is in stark contrast to AIF-necroptosis, where CAPN1 mediates AIF truncation (57 kDa) and mitochondrial release. Thus, this study suggests this exercise-induced eventuality involves AIF-necroptosis, as both activated CAPN1 and truncated-AIF were present in Dsg2^{mut/mut} myocardium. Furthermore, inhibition of CAPN1 in Dsg2^{mut/mut} ES-CMs reduced active CAPN1 levels that were accompanied by nearly absent tAIF levels. This new evidence may provide a mechanistic explanation for the findings previously reported, which demonstrated that myocyte necrosis and not apoptosis underlies cardiac dysfunction in Dsg2-mutant mice (Dsg2-N271S; homolog of human DSG2-N266S).

Among the mitochondrial assets that limit myocyte cell death, mainly when oxidatively perpetrated, is the TXN2/TXNRD2 ROS buffering system. This primary antioxidant system is under the transcriptional regulation of the Wnt/β-catenin signaling pathway, a pathway downregulated in the ACM heart. Of relevance, the CAPN system can promote 0-catenin degradation, even in the absence of external stimuli. Thus, this study reveals a novel mechanism (CAPN1 activation) further clarifying why suppression of the Wnt/β-catenin pathway, and consequentially TXN2/TXNRD2 downregulation in Dsg2^{mut/mut} mice, contributes to ACM pathogenesis. Intriguingly, CAPN1 can also truncate the negative regulator of Wnt/β-catenin signaling, glycogen synthase kinase 3β (GSK3β), at its N-terminal inhibitory domain (Serine-9, [S9]), thereby constitutively activating GSK30. It was demonstrated that GSK3β is a central contributor in ACM pathogenesis. This evidence, coupled with the fact that CAPN1 can remove S9 of GSK30, thus preventing S9-phosphorylation and GSK30-inhibition, further legitimates our recent discovery that Dsg2^{mut/mut} mice expressing constitutively active GSK3β (S9 to Alanine-9 mutation) exhibit a far worse functional and pathological phenotype compared to Dsg2^{mut/mut} mice expressing non-mutated GSK30.

Furthermore, in its active form (non-phosphorylated S9), GSK3β is known to phosphorylate either proline-rich serine/threonine (PP[S/T]×P) motifs and/or (S/T)xxxx(S/T) motifs on target substrates (where x is any amino acid). Interestingly, AIF contains both motifs in its C-terminal nuclear localization signal (NLS) domains. Thus, it is plausible that AIF may undergo GSK30-mediated phosphorylation at these domains, thus affecting AIF nuclear translocation in ACM myocytes. The current lack of publicly available phosphorylated AIF antibodies targeting such motifs defers answering this intriguing possibility to future studies. Therefore, in addition to the beneficial effects exerted by GSK30-inhibitors (see, for instance, their anti-ischemic properties), the chance of preventing further AIF post-translational modifications could also uncover novel mechanistic avenues in the relationship between GSK3β and AIF in ACM. Notwithstanding, this study reveals other post-translational modifications in AIF biology can occur, such as its oxidation. The fact that AIF is truncated and oxidized in Dsg2^{mut/mut} mice after exercise dovetails nicely with previous postulations made by others. More specifically, these authors showed that Ca²⁺ elevation activates mitochondrial CAPN1 and ROS production, the latter event resulting in oxidative modifications of AIF. Presumably, the oxidative modification of AIF augments accessibility to AIF's CAPN1 cleavage site. Translationally, genetic ablation and/or irreversible pharmacological inhibition of AIF appears to be an impracticable avenue in preventing AIF-mediated cell death because AIF is both essential and required for mitochondrial oxidative respiration. Muscle-specific loss of AIF results in mitochondrial dysfunction, muscular atrophy, and dilated cardiomyopathy. Additional in-depth studies shall determine the factors that limit mitochondrial AIF export or its posttranslational modifications.

Notwithstanding, it was showed here (a) that CAPN1 inhibition is necessary and sufficient to prevent AIF truncation in ACM myocytes; and (b) that prevention of PPIA binding to AIF using an AIF-TAT mimetic peptide is efficacious in preventing apoptosis, nuclear localization of tAIF, and nuclear loss of HMGB1 (a bona fide marker of cell necrosis).

Limitations and Studies in Perspective

The TXN system does not fully portray the antioxidant armamentarium, both in mitochondria and the cytosol. However, it is suspected that glutathione (GSH) could also be affected in ACM cardiomyocytes at rest and even more so after chronic exercise. Secondly, calpains can disrupt ATP synthase inducing superoxide production or participate in MPT pore induction. These eventualities can also contribute to ACM pathogenesis and deserve future, in-depth investigation.

Yet, this approach was chosen for three main reasons. First, isolation of adult cardiomyocytes primarily results in single-cell isolation with infrequent paired myocytes (<1-2% of isolation culture); thus, primary adult cardiomyocytes would not be the ideal bench-test when studying a disease in which cell-cell mechanical/electrical contact is dependent on desmosomal integrity. Utilizing cell monolayers (>90% confluence) provided an assessment of apoptotic vs. necrotic cellular morphology and the ability of ISO/Ca²⁺ to disrupt these cellular contacts. Second, infiltrative CD68+ macrophages and CD3+ T-cells are present in the hearts of Dsg2^{mut/mut} mice; thus, the presence of professional immune cells isolated from adult Dsg2^{mut/mut} cardiac perfusates would have confounded our outcomes. Notably, in light of previous studies that showed the regulatory role of CAPN1 in T-cell proliferation. The use of ES-CM cultures provided an ideal surrogate, as this system guarantees >95-99% cardiomyocyte purity/population and is thus devoid of immune cells. Third, utilization of primary adult myocytes longer than 72 hrs was unfeasible in the present case; this limitation would have precluded the possibility of testing the impact of sustained ISO/Ca²⁺ challenge up to 7 days, as was done here. It was previously demonstrated that the amount and intensity of exercise increase disease penetrance in patients with ACM. Thus, it was paramount for us to compare the impact of acute (1 day) vs. chronic (7 days) β-adrenergic/Ca²⁺ challenge in ACM ES-CMs.

Evidence demonstrating AIF+ nuclei in the myocardium of DCM, HCM, and IHD subjects, albeit lower than ACM samples, suggests that AIF nuclear translocation (and consequent adverse effects) may still account for myocyte cell death in other forms of cardiomyopathies. On the other hand, this evidence reiterates the necessity of correlating histopathological findings with clinical history, such as exercise training or other forms of physiological or pathological stressors.

Current evidence obtained in a small group (six) of G+/P+ ACM individuals who all harbor a pathogenic PKP2 mutation showed a correlation between AIF scores with METhrs (i.e., exercise history). Future, dedicated studies shall include a more significant number of retrospective exercise participation reports from ACM patients for which myocardial samples have been collected. However, this limitation does not detract from, instead supports the current mechanistic notion that AIF-mediated myocyte death is more likely to occur in the presence of a pathological “substrate” (e.g., desmosomal mutation) and an environmental “trigger” (e.g., exercise and/or hemodynamic stress). Finally, it is tempting to extend a similar pathogenic scenario (Ca²⁺/CAPN1/TXN2/PPIA/AIF) to other cell types, including cardiac sympathetic neurons and/or mesenchymal cells, that may account for other ACM adversities (i.e., arrhythmias). Therefore, heart rhythm disorders may descend from a combination of abnormal Ca²⁺/CAPN1/TXN2/PPIA/AIF handling, myocardium loss, and/or neuronal/mesenchymal cell death or sympathetic nerve hyper innervation.

### CONCLUSIONS

This study reveals a means by which exercise induces cell necroptosis in ACM that is mediated by a Ca²⁺/CAPN1/TXN2/PPIA/AIF system (FIG. 20), offering unprecedented opportunities to deepen our understanding of ACM pathophysiology. Although the present mechanism unlikely exhausts all the possibilities underpinning exercise-instigated myocyte loss, ACM appears to be a nicely suited example of AIF-induced myocyte necroptosis. Moreover, the adverse consequences of CAPN1/AIF activation and PPIA/AIF-complex binding could be at play in other forms of cardiomyopathies characterized by high myocardial cell death levels and fibrotic remodeling, such as myocarditis, and with a clinical history of either sustained physiological or pathological stressors. Thus, current findings expand beyond existing paradigms of disease mechanisms triggered in ACM and may provide a novel therapeutic approach in the prevention of disease onset and progression in ACM individuals and subjects affected by other forms of cardiomyopathies of inherited or acquired nature.

As shown in the top panel (Healthy Heart, FIG. 20), cytosolic AIF (67 kDa; amino acids: ΔMethionine [M1]—Aspartic Acid [D613]) is in its immature (iAIF) form when imported into the mitochondria. Where iAIF is cleaved by a mitochondrial processing peptidase (MPP) to form mature AIF (mAIF) where it is bound to the inner mitochondrial membrane (IMM). Mitochondrial-bound mAIF (62 kDa; amino acids: ΔAlanine [A54]-D613) acts as NADH-oxidoreductase recycling reducing equivalents (i.e., NADH/NAD+) back into the electron transport chain and additionally aids in Complex-I (CI) respiratory subunit assembly. Superoxide molecules (O₂) generated by CI and Complex-III (CIII) are converted to hydrogen peroxide (H₂O₂) via dismutation. Where in healthy mitochondria, hydrogen peroxide is reduced to water (H₂O) via the thioredoxin-2 (TXN2), TXN2-reductase (TXNRD2), peroxiredoxin-3 (PRXD3) system to prevent the formation of reactive oxygen species (ROS). As shown in the lower panel (Arrhythmogenic Cardiomyopathy) (I), a depleted TXN2/TXNRD2/PRXD3 anti-ROS buffering system results in the accumulation of ROS, (II) where cysteine residues of mAIF are oxidized (—C-Ox) by augmented ROS levels. (III) Additionally, excessive ROS accumulation leads to permeabilization (intermittent dashed line) of the outer mitochondrial membrane (OMM), leading to outward solute leakage and inward calcium (Ca²⁺) pumping. (IV) AIF undergoes a conformational change upon cysteine-oxidation, promoting Ca²⁺-activated CAPN1 cleavage of Ox-mAIF to form truncated AIF (tAIF; 57 kDa; amino acids: ΔLeucine [L103]-D613). (V) Oxidized, truncated AIF (Ox-tAIF) is liberated into the cytosol where cyclophilin-A (PPIA) binds to Ox-tAIF. (VI) PPIA-bound tAIF is transported to the myocyte nucleus where it causes large scale DNA fragmentation and nuclear loss of HMGB1. (VII) Utilizing an AIF-TAT mimetic peptide that mirrors the binding site of PPIA with a strong affinity to PPIA-binding (K_{D}=1.2×10⁻⁵ M), (IIX) PPIA-mediated nuclear transport of tAIF can be prevented. Thereby inhibiting AIF-induced cell death, including apoptosis, necrosis and necroptosis.

Although the invention has been described with reference to the above examples, it will be understood that modifications and variations are encompassed within the spirit and scope of the invention. Accordingly, the invention is limited only by the following claims.

SEQUENCES:

| Sequence | SEQ ID NO |
|---|---|
| CLKLKLLRKVC | SEQ ID NO: 1 |
| CLKIKLLRKVC | SEQ ID NO: 2 |
| CLKIKLLRKVC | SEQ ID NO: 3 |
| CLKIKLLRKIC | SEQ ID NO: 4 |
| CLKIKLLRK(Tle)C | SEQ ID NO: 5 |
| C(Tle)K(Tle)KLLRK(Tle)C | SEQ ID NO: 6 |
| C(Tle)KLKLLRK(Tle)C | SEQ ID NO: 7 |
| C(Tle)KLKLLRKVC | SEQ ID NO: 8 |
| C(Tle)(hR)L(hR)LL(hR)KVC | SEQ ID NO: 9 |
| C(hBL)KLKLLRKVC | SEQ ID NO: 10 |
| C(hBL)K(hBL)KLLRKVC | SEQ ID NO: 11 |
| C(hBL)K(hBL)KLLRK(hßL)C | SEQ ID NO: 12 |
| C(hBL)KLKLLRKVC | SEQ ID NO: 13 |
| C(hBI)K(hBI)KLLRKVC | SEQ ID NO: 14 |
| C(hBI)K(hBI)KLLRK(hBI)C | SEQ ID NO: 15 |
| C(Nle)KLKLLRKVC | SEQ ID NO: 16 |
| C(Nle)K(Nle)KLLRKVC | SEQ ID NO: 17 |
| C(Nle)K(Nle)KLLRK(Nle)C | SEQ ID NO: 18 |
| CI(Orn)LKLLRKVC | SEQ ID NO: 19 |
| CI(Orn)L(Orn)LLRKVC | SEQ ID NO: 20 |
| CI(Orn)L(Orn)LL(Orn)KVC | SEQ ID NO: 21 |
| CI(Orn)L(Orn)LL(Orn) | SEQ ID NO: 22 |
| (Orn)VC | |
| CIHLKLLRKVC | SEQ ID NO: 23 |
| CIHLHLLRKVC | SEQ ID NO: 24 |
| CIHLHLLHKVC | SEQ ID NO: 25 |
| CIHLHLLHHVC | SEQ ID NO: 26 |
| CIKLK(Aib)LRKVC | SEQ ID NO: 27 |
| CIKLK(Aib)(Aib)RKVC | SEQ ID NO: 28 |
| CIKLKL(Aib)RKVC | SEQ ID NO: 29 |
| CIKLKPPRKVC | SEQ ID NO: 30 |
| RLLPGFMCQGGDFTR | SEQ ID NO: 31 |
| RIIPGFMCQGGDFT(hR) | SEQ ID NO: 32 |
| (hR)IIPGFMCQGGDFT(hR) | SEQ ID NO: 33 |
| RIIPGFMCQGGDFTH | SEQ ID NO: 34 |
| RIIPGFMCQGGDFTK | SEQ ID NO: 35 |
| RIIPGFMCEGGDFTK | SEQ ID NO: 36 |
| RIIPGFMCEGGDFTK | SEQ ID NO: 37 |
| RIIPGFM(SPRC)EGGDFTK | SEQ ID NO: 38 |
| RIIPGFMCQGGDWTR | SEQ ID NO: 39 |
| RIIPGWMCQGGDWTR | SEQ ID NO: 40 |
| RIIPGWMCQGGDFTR | SEQ ID NO: 41 |
| RIIPGWMCQGGDWTR | SEQ ID NO: 42 |
| KIIPGWMCQGGDWTE | SEQ ID NO: 43 |
| KIIPGFMCQGGDFTE | SEQ ID NO: 44 |
| (Orn)IIPGFMCQGGDFTD | SEQ ID NO: 45 |
| R(Nle)(Nle)PGFMCQGGDFTR | SEQ ID NO: 46 |
| RI(Nle)PGFMCQGGDFTR | SEQ ID NO: 47 |
| R(Tle)(Tle)PGFMCQGGDFTR | SEQ ID NO: 48 |
| RII(PCA)GFMCQGGDFTR | SEQ ID NO: 49 |
| KII(PCA)GFMCQGGDFTE | SEQ ID NO: 50 |
| KII(PCA)GFMCQGGDFTD | SEQ ID NO: 51 |
| KIIPGWMCQGGDWTD | SEQ ID NO: 52 |
| (Orn)IIPGFMCQGGDFTE | SEQ ID NO: 53 |
| (Orn)IIPGWMCQGGDWTE | SEQ ID NO: 54 |
| (Orn)IIPGWMCQGGDWTD | SEQ ID NO: 55 |
| KIIPGWMCQGGDWTD | SEQ ID NO: 56 |
| HIIPGFMCQGGDFTH | SEQ ID NO: 57 |
| HIIPGWMCQGGDWTH | SEQ ID NO: 58 |
| KIIPGFMCQGGDFTK | SEQ ID NO: 59 |
| KIIPGWMCQGGDWTK | SEQ ID NO: 60 |
| Ac-QSVGVSSGKLLIKLK_{D} | SEQ ID NO: 61 |
| GRKVETDHI-NH₂ | |
| H₂-GRKKRRQRRR(βAla)QSVGV | SEQ ID NO: 62 |
| SSGKLLIKLK_{D}GRKVETDHI-NH₂ | |
| Ac-QSCGVSSG[K(N₃)]LLCKLK | SEQ ID NO: 63 |
| DGRKV[hpG]TDHI-NH₂ | |
| Ac-RIIPGFMCQGGDFTR-NH₂ | SEQ ID NO: 64 |
| Ac-CIKLK_{D}GRK VC-NH₂ | SEQ ID NO: 65 |
| Ac-CIKLKLLRKVC-NH₂ | SEQ ID NO: 66 |
| CIKLK_{D}GRKVC | SEQ ID NO: 67 |
| IKLK_{D}GRKV | SEQ ID NO: 68 |
| RIIPGFMCQGGDFTR | SEQ ID NO: 69 |
| RQIKIWFQNRRMKWKK | SEQ ID NO: 70 |
| GRKKRRQRRRPPQ | SEQ ID NO: 71 |
| LLIILRRRIRKQAHAHSK | SEQ ID NO: 72 |
| GWTLNSAGYLLGKINLKA | SEQ ID NO: 73 |
| LAALAKKIL | |
| GALFLGFLGAAGSTMGAW | SEQ ID NO: 74 |
| SPKKKRKV | |
| KETWWETWWTEWSQPKKKRKV | SEQ ID NO: 75 |
| RRRRRRRR | SEQ ID NO: 76 |
| RRRRRRRRR | SEQ ID NO: 77 |
| KLALKLALKALKAALKLA | SEQ ID NO: 78 |
| RRWWRRWRR | SEQ ID NO: 79 |
| LSTAADMQGVVTDGMASGLDK | SEQ ID NO: 80 |
| DYLKPDD | |

1. An isolated peptide having an amino acid sequence as set forth in Formula I:
Y₁—C—X₁—X₂—X₃—X₄—X₅—X₆—X₇—X₈—X₉—C—Y₂ (Formula I),
wherein:
Y₁ is a cell penetrating peptide (CPP), a hydrogen atom or an acetyl group;
Y₂ is a CPP or a hydrogen atom;
C is a cysteine;
X₁ is isoleucine, leucine, valine, terleucine, norleucine, beta-homoleucine, beta-homoisoleucine or 3-amino-5,5-dimethylhexanoic acid in either R or S absolute configuration;
X₂ is lysine, histidine, arginine, homoarginine, ornithine, 2,3-diaminopropionic acid, 2,4-diaminobutyric acid or 2-amino-3-guanidinopropionic acid in either R or S absolute configuration;
X₃ is leucine, isoleucine, leucine, valine, terleucine, norleucine, beta-homoleucine, beta-homoisoleucine or 3-amino-5,5-dimethylhexanoic acid in either R or S absolute configuration;
X₄ is lysine, histidine, arginine, homoarginine, ornithine, 2,3-diaminopropionic acid, 2,4-diaminobutyric acid or 2-amino-3-guanidinopropionic acid in either R or S absolute configuration;
X₅ is aspartic acid, isoleucine, leucine, valine, terleucine, norleucine, beta-homoleucine, beta-homoisoleucine, 3-amino-5,5-dimethylhexanoic acid, proline, nipecotic acid, piperidine-2-carboxylic acid, piperidine-4-carboxylic acid, 1,2-dihydro-3(6h)-pyridinone beta-alanine, 2-aminoisobutyric acid, glycine, asparagine, tryptophan or phenylalanine in either R or S absolute configuration;
X₆ is glycine, isoleucine, leucine, valine, terleucine, norleucine, beta-homoleucine, beta-homoisoleucine, 3-amino-5,5-dimethylhexanoic acid, proline, nipecotic acid, piperidine-2-carboxylic acid, piperidine-4-carboxylic acid, 1,2-dihydro-3(6h)-pyridinone beta-alanine, 2 aminoisobutyric acid, glycine, asparagine, tryptophan or phenylalanine in either R or S absolute configuration;
X₇ is arginine, histidine, lysine, homoarginine, ornithine, 2,3-diaminopropionic acid, 2,4-diaminobutyric acid, or 2-amino-3-guanidinopropionic acid in either R or S absolute configuration;
X₈ is lysine, histidine, arginine, homoarginine, ornithine, 2,3-diaminopropionic acid, 2,4-diaminobutyric acid or 2-amino-3-guanidinopropionic acid in either R or S absolute configuration; and
X₉ is valine, isoleucine, leucine, terleucine, norleucine, beta-homoleucine, beta-homoisoleucine or 3-amino-5,5-dimethylhexanoic acid in either R or S absolute configuration;
or as set forth in Formula II:
Y₃—X₁₀—X₁₁—X₁₂—X₁₃—X₁₄—X₁₅—X₁₆—X₁₇—X₁₈—X₁₉—X₂—X₂₁—X₂₂—X₂₃—X₂₄—Y₄ (Formula II),
wherein:
Y₃ is a CPP, a hydrogen atom or an acetyl group;
Y₄ is a CPP, a hydroxyl group or an amino group;
X₁₀ is arginine, histidine, lysine, glutamic acid, glutamine, aspartic acid, homoarginine, ornithine, 2,3-diaminopropionic acid, 2,4-diaminobutyric acid or 2-amino-3-guanidinopropionic acid in either R or S absolute configuration;
X₁₁ is isoleucine, leucine, valine, terleucine, norleucine, beta-homoleucine, beta-homoisoleucine or 3-amino-5,5-dimethylhexanoic acid in either R or S absolute configuration;
X₁₂ is isoleucine, leucine, valine, tert-leucine, norleucine, beta-homoleucine, beta-homoisoleucine; or 3-amino-5,5-dimethylhexanoic acid in either r or s absolute configuration;
x₁₃ is proline, 1,2,3,4-tetrahydroisoquinoline-3-carboxylic acid or pyroglutamic acid in either R or S absolute configuration.
X₁₄ is glycine;
X₁₅ is phenylalanine, tyrosine, tryptophan in either R or S absolute configuration;
X₁₆ is methionine, cysteine, penicillamine, or s-propargyl-cysteine in either R or S absolute configuration;
X₁₇ is cysteine, penicillamine, methionine, or s-propargyl-cysteine in either R or S absolute configuration;
X₁₈ is glutamine, asparagine, glutamic acid, or aspartic acid in either R or S absolute configuration;
X₁₉ is glycine;
X₂₀ is glycine;
X₂₁ is aspartic acid, glutamic acid arginine, histidine or lysine in either R or S absolute configuration;
X₂₂ is phenylalanine, tryptophan or tyrosine in either R or S absolute configuration;
X₂₃ is threonine or serine in either R or S absolute configuration; and
X₂₄ is arginine, histidine, lysine, glutamic acid, aspartic acid, glutamine, homoarginine, ornithine, 2,3-diaminopropionic acid, 2,4-diaminobutyric acid or 2-amino-3-guanidinopropionic acid in either R or S absolute configuration.

2. The isolated peptide of claim 1, wherein the peptide has an amino acid sequence as set forth in any of SEQ ID NOs:1-60.

3. The isolated peptide of claim 1, wherein the isolated peptide comprises an N-terminal modification, a C-terminal modification, a detectable label, a cell-penetrating peptide (CPP), a non-natural amino acid, a cyclic peptide, or a combination thereof.

4. The isolated peptide of claim 3, wherein the CPP improves cellular uptake, cell penetration and/or transport of the peptide.

5. The isolated peptide of claim 3, wherein the CPP is selected from the group consisting of transactivator of transcription (TAT) peptide and TAT peptide variants.

6. The isolated peptide of claim 5, wherein the TAT peptide is human immunodeficiency virus TAT.

7. The isolated peptide of claim 1, wherein the peptide binds to apoptosis-inducing factor (AIF) and/or to peptidyl-prolyl cis-trans isomerase (PPIA).

8. The isolated peptide of claim 7, wherein the peptide is an AIF mimetic peptide.

9. The isolated peptide of claim 7, wherein the peptide inhibits Ca²⁺-Calpain-1 (CAPN1)-induced cell death in myocytes.

10-13. (canceled)

14. An isolated nucleic acid sequence encoding a peptide of claim 1.

15. A pharmaceutical composition comprising an isolated peptide of claim 1 and a pharmaceutically acceptable carrier.

16-17. (canceled)

18. The pharmaceutical composition of claim 17, wherein the CPP is human immunodeficiency virus transactivator of transcription (TAT) peptide.

19. The pharmaceutical composition of claim 15, wherein the pharmaceutically acceptable carrier is selected from the group consisting of phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; sucrose; mannitol; trehalose; sorbitol; sodium; saline; metal surfactants; non-ionic surfactants; polyethylene glycol (PEG); magnesium stearate; water; alcohol; saline solution; glycol; mineral oil and dimethyl sulfoxide (DMSO).

20. A method of preventing myocardial cell death and/or sudden cardiac death in a subject comprising administering to a subject in need thereof a therapeutically effective amount of the pharmaceutical composition of claim 15, thereby preventing myocardial cell death and/or sudden cardiac death.

21. The method of claim 20, wherein the isolated peptide has an amino acid sequence as set forth in any of SEQ ID NOs:1-60.

22. The method of claim 21, wherein the peptide comprises an N-terminal modification, a C-terminal modification, a detectable label, a cell-penetrating peptide (CPP), a non-natural amino acid, a cyclic peptide, or a combination thereof.

23. The pharmaceutical composition of claim 22, wherein the CPP is human immunodeficiency virus transactivator of transcription (TAT) peptide.

24-27. (canceled)

28. The method of claim 20, wherein the peptide binds to apoptosis-inducing factor (AIF) and/or to peptidyl-prolyl cis-trans isomerase (PPIA).

29. The method of claim 28, wherein the peptide is an AIF mimetic peptide.

30. The method of claim 28, wherein the peptide inhibits Ca²⁺-Calpain-1 (CAPN1)-induced cell death in myocytes.

31-33. (canceled)

34. The method of claim 25, further comprising administering to the subject an additional therapeutic treatment.

35. The method of claim 34, wherein the additional therapeutic treatment comprises a beta-blocker, an antiarrhythmic agent, an anticoagulant, or an implantable cardioverter-defibrillator.


SEQUENCE LISTING (82 sequences)

SEQ ID NO: 1 - protein, 11 aa, Artificial Sequence
  Description of Artificial Sequence Synthetic peptide
CLKLKLLRKVC

SEQ ID NO: 2 - protein, 11 aa, Artificial Sequence
  Description of Artificial Sequence Synthetic peptide
CLKIKLLRKVC

SEQ ID NO: 3 - protein, 11 aa, Artificial Sequence
  Description of Artificial Sequence Synthetic peptide
CLKIKLLRKVC

SEQ ID NO: 4 - protein, 11 aa, Artificial Sequence
  Description of Artificial Sequence Synthetic peptide
CLKIKLLRKIC

SEQ ID NO: 5 - protein, 11 aa, Artificial Sequence
  Description of Artificial Sequence Synthetic peptide
  MOD_RES (10)..(10): terleucine
CLKIKLLRKLC

SEQ ID NO: 6 - protein, 11 aa, Artificial Sequence
  Description of Artificial Sequence Synthetic peptide
  MOD_RES (2)..(2): terleucine
  MOD_RES (4)..(4): terleucine
  MOD_RES (10)..(10): terleucine
CLKLKLLRKLC

SEQ ID NO: 7 - protein, 11 aa, Artificial Sequence
  Description of Artificial Sequence Synthetic peptide
  MOD_RES (2)..(2): terleucine
  MOD_RES (10)..(10): terleucine
CLKLKLLRKLC

SEQ ID NO: 8 - protein, 11 aa, Artificial Sequence
  Description of Artificial Sequence Synthetic peptide
  MOD_RES (2)..(2): terleucine
CLKLKLLRKVC

SEQ ID NO: 9 - protein, 11 aa, Artificial Sequence
  Description of Artificial Sequence Synthetic peptide
  MOD_RES (2)..(2): terleucine
  MOD_RES (3)..(3): homoarginine
  MOD_RES (5)..(5): homoarginine
  MOD_RES (8)..(8): homoarginine
CLRLRLLRKVC

SEQ ID NO: 10 - protein, 11 aa, Artificial Sequence
  Description of Artificial Sequence Synthetic peptide
  MOD_RES (2)..(2): beta-homoleucine
CLKLKLLRKVC

SEQ ID NO: 11 - protein, 11 aa, Artificial Sequence
  Description of Artificial Sequence Synthetic peptide
  MOD_RES (2)..(2): beta-homoleucine
  MOD_RES (4)..(4): beta-homoleucine
CLKLKLLRKVC

SEQ ID NO: 12 - protein, 11 aa, Artificial Sequence
  Description of Artificial Sequence Synthetic peptide
  MOD_RES (2)..(2): beta-homoleucine
  MOD_RES (4)..(4): beta-homoleucine
  MOD_RES (10)..(10): beta-homoleucine
CLKLKLLRKLC

SEQ ID NO: 13 - protein, 11 aa, Artificial Sequence
  Description of Artificial Sequence Synthetic peptide
  MOD_RES (2)..(2): beta-homoleucine
CLKLKLLRKVC

SEQ ID NO: 14 - protein, 11 aa, Artificial Sequence
  Description of Artificial Sequence Synthetic peptide
  MOD_RES (2)..(2): beta-homoisoleucine
  MOD_RES (4)..(4): beta-homoisoleucine
CIKIKLLRKVC

SEQ ID NO: 15 - protein, 11 aa, Artificial Sequence
  Description of Artificial Sequence Synthetic peptide
  MOD_RES (2)..(2): beta-homoisoleucine
  MOD_RES (4)..(4): beta-homoisoleucine
  MOD_RES (10)..(10): beta-homoisoleucine
CIKIKLLRKIC

SEQ ID NO: 16 - protein, 11 aa, Artificial Sequence
  Description of Artificial Sequence Synthetic peptide
  MOD_RES (2)..(2): norleucine
CLKLKLLRKVC

SEQ ID NO: 17 - protein, 11 aa, Artificial Sequence
  Description of Artificial Sequence Synthetic peptide
  MOD_RES (2)..(2): norleucine
  MOD_RES (4)..(4): norleucine
CLKLKLLRKVC

SEQ ID NO: 18 - protein, 11 aa, Artificial Sequence
  Description of Artificial Sequence Synthetic peptide
  MOD_RES (2)..(2): norleucine
  MOD_RES (4)..(4): norleucine
  MOD_RES (10)..(10): norleucine
CLKLKLLRKLC

SEQ ID NO: 19 - protein, 11 aa, Artificial Sequence
  Description of Artificial Sequence Synthetic peptide
  MOD_RES (3)..(3): ornithine
CIXLKLLRKVC

SEQ ID NO: 20 - protein, 11 aa, Artificial Sequence
  Description of Artificial Sequence Synthetic peptide
  MOD_RES (3)..(3): ornithine
  MOD_RES (5)..(5): ornithine
CIXLXLLRKVC

SEQ ID NO: 21 - protein, 11 aa, Artificial Sequence
  Description of Artificial Sequence Synthetic peptide
  MOD_RES (3)..(3): ornithine
  MOD_RES (5)..(5): ornithine
  MOD_RES (8)..(8): ornithine
CIXLXLLXKVC

SEQ ID NO: 22 - protein, 11 aa, Artificial Sequence
  Description of Artificial Sequence Synthetic peptide
  MOD_RES (3)..(3): ornithine
  MOD_RES (5)..(5): ornithine
  MOD_RES (8)..(8): ornithine
  MOD_RES (9)..(9): ornithine
CIXLXLLXXVC

SEQ ID NO: 23 - protein, 11 aa, Artificial Sequence
  Description of Artificial Sequence Synthetic peptide
CIHLKLLRKVC

SEQ ID NO: 24 - protein, 11 aa, Artificial Sequence
  Description of Artificial Sequence Synthetic peptide
CIHLHLLRKVC

SEQ ID NO: 25 - protein, 11 aa, Artificial Sequence
  Description of Artificial Sequence Synthetic peptide
CIHLHLLHKVC

SEQ ID NO: 26 - protein, 11 aa, Artificial Sequence
  Description of Artificial Sequence Synthetic peptide
CIHLHLLHHVC

SEQ ID NO: 27 - protein, 11 aa, Artificial Sequence
  Description of Artificial Sequence Synthetic peptide
  MOD_RES (6)..(6): 2-aminoisobutyric acid
CIKLKXLRKVC

SEQ ID NO: 28 - protein, 11 aa, Artificial Sequence
  Description of Artificial Sequence Synthetic peptide
  MOD_RES (6)..(6): 2-aminoisobutyric acid
  MOD_RES (7)..(7): 2-aminoisobutyric acid
CIKLKXXRKVC

SEQ ID NO: 29 - protein, 11 aa, Artificial Sequence
  Description of Artificial Sequence Synthetic peptide
  MOD_RES (7)..(7): 2-aminoisobutyric acid
CIKLKLXRKVC

SEQ ID NO: 30 - protein, 11 aa, Artificial Sequence
  Description of Artificial Sequence Synthetic peptide
CIKLKPPRKVC

SEQ ID NO: 31 - protein, 15 aa, Artificial Sequence
  Description of Artificial Sequence Synthetic peptide
RLLPGFMCQGGDFTR

SEQ ID NO: 32 - protein, 15 aa, Artificial Sequence
  Description of Artificial Sequence Synthetic peptide
  MOD_RES (15)..(15): homoarginine
RIIPGFMCQGGDFTR

SEQ ID NO: 33 - protein, 15 aa, Artificial Sequence
  Description of Artificial Sequence Synthetic peptide
  MOD_RES (1)..(1): homoarginine
  MOD_RES (15)..(15): homoarginine
RIIPGFMCQGGDFTR

SEQ ID NO: 34 - protein, 15 aa, Artificial Sequence
  Description of Artificial Sequence Synthetic peptide
RIIPGFMCQGGDFTH

SEQ ID NO: 35 - protein, 15 aa, Artificial Sequence
  Description of Artificial Sequence Synthetic peptide
RIIPGFMCQGGDFTK

SEQ ID NO: 36 - protein, 15 aa, Artificial Sequence
  Description of Artificial Sequence Synthetic peptide
RIIPGFMCEGGDFTK

SEQ ID NO: 37 - protein, 15 aa, Artificial Sequence
  Description of Artificial Sequence Synthetic peptide
RIIPGFMCEGGDFTK

SEQ ID NO: 38 - protein, 15 aa, Artificial Sequence
  Description of Artificial Sequence Synthetic peptide
  MOD_RES (8)..(8): s-propargyl-cysteine
RIIPGFMCEGGDFTK

SEQ ID NO: 39 - protein, 15 aa, Artificial Sequence
  Description of Artificial Sequence Synthetic peptide
RIIPGFMCQGGDWTR

SEQ ID NO: 40 - protein, 15 aa, Artificial Sequence
  Description of Artificial Sequence Synthetic peptide
RIIPGWMCQGGDWTR

SEQ ID NO: 41 - protein, 15 aa, Artificial Sequence
  Description of Artificial Sequence Synthetic peptide
RIIPGWMCQGGDFTR

SEQ ID NO: 42 - protein, 15 aa, Artificial Sequence
  Description of Artificial Sequence Synthetic peptide
RIIPGWMCQGGDWTR

SEQ ID NO: 43 - protein, 15 aa, Artificial Sequence
  Description of Artificial Sequence Synthetic peptide
KIIPGWMCQGGDWTE

SEQ ID NO: 44 - protein, 15 aa, Artificial Sequence
  Description of Artificial Sequence Synthetic peptide
KIIPGFMCQGGDFTE

SEQ ID NO: 45 - protein, 15 aa, Artificial Sequence
  Description of Artificial Sequence Synthetic peptide
  MOD_RES (1)..(1): ornithine
XIIPGFMCQGGDFTD

SEQ ID NO: 46 - protein, 15 aa, Artificial Sequence
  Description of Artificial Sequence Synthetic peptide
  MOD_RES (2)..(2): norleucine
  MOD_RES (3)..(3): norleucine
RLLPGFMCQGGDFTR

SEQ ID NO: 47 - protein, 15 aa, Artificial Sequence
  Description of Artificial Sequence Synthetic peptide
  MOD_RES (3)..(3): norleucine
RILPGFMCQGGDFTR

SEQ ID NO: 48 - protein, 15 aa, Artificial Sequence
  Description of Artificial Sequence Synthetic peptide
  MOD_RES (2)..(2): terleucine
  MOD_RES (3)..(3): terleucine
RLLPGFMCQGGDFTR

SEQ ID NO: 49 - protein, 15 aa, Artificial Sequence
  Description of Artificial Sequence Synthetic peptide
  MOD_RES (4)..(4): pyroglutamic acid
RIIEGFMCQGGDFTR

SEQ ID NO: 50 - protein, 15 aa, Artificial Sequence
  Description of Artificial Sequence Synthetic peptide
  MOD_RES (4)..(4): pyroglutamic acid
KIIEGFMCQGGDFTE

SEQ ID NO: 51 - protein, 15 aa, Artificial Sequence
  Description of Artificial Sequence Synthetic peptide
  MOD_RES (4)..(4): pyroglutamic acid
KIIEGFMCQGGDFTD

SEQ ID NO: 52 - protein, 15 aa, Artificial Sequence
  Description of Artificial Sequence Synthetic peptide
KIIPGWMCQGGDWTD

SEQ ID NO: 53 - protein, 15 aa, Artificial Sequence
  Description of Artificial Sequence Synthetic peptide
  MOD_RES (1)..(1): ornithine
XIIPGFMCQGGDFTE

SEQ ID NO: 54 - protein, 15 aa, Artificial Sequence
  Description of Artificial Sequence Synthetic peptide
  MOD_RES (1)..(1): ornithine
XIIPGWMCQGGDWTE

SEQ ID NO: 55 - protein, 15 aa, Artificial Sequence
  Description of Artificial Sequence Synthetic peptide
  MOD_RES (1)..(1): ornithine
XIIPGWMCQGGDWTD

SEQ ID NO: 56 - protein, 15 aa, Artificial Sequence
  Description of Artificial Sequence Synthetic peptide
KIIPGWMCQGGDWTD

SEQ ID NO: 57 - protein, 15 aa, Artificial Sequence
  Description of Artificial Sequence Synthetic peptide
HIIPGFMCQGGDFTH

SEQ ID NO: 58 - protein, 15 aa, Artificial Sequence
  Description of Artificial Sequence Synthetic peptide
HIIPGWMCQGGDWTH

SEQ ID NO: 59 - protein, 15 aa, Artificial Sequence
  Description of Artificial Sequence Synthetic peptide
KIIPGFMCQGGDFTK

SEQ ID NO: 60 - protein, 15 aa, Artificial Sequence
  Description of Artificial Sequence Synthetic peptide
KIIPGWMCQGGDWTK

SEQ ID NO: 61 - protein, 25 aa, Artificial Sequence
  Description of Artificial Sequence Synthetic peptide
QSVGVSSGKLLIKLKDGRKVETDHI

SEQ ID NO: 62 - protein, 36 aa, Artificial Sequence
  Description of Artificial Sequence Synthetic polypeptide
  MOD_RES (11)..(11): 1,2-dihydro-3(6h)-pyridinone beta-alanine
GRKKRRQRRRAQSVGVSSGKLLIKLKDGRKVETDHI

SEQ ID NO: 63 - protein, 25 aa, Artificial Sequence
  Description of Artificial Sequence Synthetic peptide
  MOD_RES (21)..(21): 1-homopropargylglycine
QSCGVSSGKLLCKLKDGRKVGTDHI

SEQ ID NO: 64 - protein, 15 aa, Artificial Sequence
  Description of Artificial Sequence Synthetic peptide
RIIPGFMCQGGDFTR

SEQ ID NO: 65 - protein, 11 aa, Artificial Sequence
  Description of Artificial Sequence Synthetic peptide
CIKLKDGRKVC

SEQ ID NO: 66 - protein, 11 aa, Artificial Sequence
  Description of Artificial Sequence Synthetic peptide
CIKLKLLRKVC

SEQ ID NO: 67 - protein, 11 aa, Artificial Sequence
  Description of Artificial Sequence Synthetic peptide
CIKLKDGRKVC

SEQ ID NO: 68 - protein, 9 aa, Artificial Sequence
  Description of Artificial Sequence Synthetic peptide
IKLKDGRKV

SEQ ID NO: 69 - protein, 15 aa, Artificial Sequence
  Description of Artificial Sequence Synthetic peptide
RIIPGFMCQGGDFTR

SEQ ID NO: 70 - protein, 16 aa, Artificial Sequence
  Description of Artificial Sequence Synthetic peptide
RQIKIWFQNRRMKWKK

SEQ ID NO: 71 - protein, 13 aa, Artificial Sequence
  Description of Artificial Sequence Synthetic peptide
GRKKRRQRRRPPQ

SEQ ID NO: 72 - protein, 18 aa, Artificial Sequence
  Description of Artificial Sequence Synthetic peptide
LLIILRRRIRKQAHAHSK

SEQ ID NO: 73 - protein, 27 aa, Artificial Sequence
  Description of Artificial Sequence Synthetic peptide
GWTLNSAGYLLGKINLKALAALAKKIL

SEQ ID NO: 74 - protein, 26 aa, Artificial Sequence
  Description of Artificial Sequence Synthetic peptide
GALFLGFLGAAGSTMGAWSPKKKRKV

SEQ ID NO: 75 - protein, 21 aa, Artificial Sequence
  Description of Artificial Sequence Synthetic peptide
KETWWETWWTEWSQPKKKRKV

SEQ ID NO: 76 - protein, 8 aa, Artificial Sequence
  Description of Artificial Sequence Synthetic peptide
RRRRRRRR

SEQ ID NO: 77 - protein, 9 aa, Artificial Sequence
  Description of Artificial Sequence Synthetic peptide
RRRRRRRRR

SEQ ID NO: 78 - protein, 18 aa, Artificial Sequence
  Description of Artificial Sequence Synthetic peptide
KLALKLALKALKAALKLA

SEQ ID NO: 79 - protein, 9 aa, Artificial Sequence
  Description of Artificial Sequence Synthetic peptide
RRWWRRWRR

SEQ ID NO: 80 - protein, 28 aa, Artificial Sequence
  Description of Artificial Sequence Synthetic peptide
LSTAADMQGVVTDGMASGLDKDYLKPDD

SEQ ID NO: 81 - protein, 4 aa, Artificial Sequence
  Description of Artificial Sequence Synthetic peptide
LLVY

SEQ ID NO: 82 - protein, 12 aa, Artificial Sequence
  Description of Artificial Sequence Synthetic peptide
  SITE (1)..(12): This sequence may encompass 6-12 residues
RRRRRRRRRRRR